(19) United States
(12) Patent Application Publication
(10) Pub. No.: US 2022/0204654 A1
(43) Pub. Date: Jun. 30, 2022

(54) CYCLODEXTRIN SUPRAMOLECULAR SCAFFOLDS AND USES THEREOF

(71) Applicant: Massachusetts Institute of Technology, Cambridge, MA (US)

(72) Inventors: Chandrabali Bhattacharya, Cambridge, MA (US); Robert S. Langer, Newton, MA (US); Daniel Griffith Anderson, Framingham, MA (US); Arijit Basu, Quincy, MA (US)

(73) Assignee: Massachusetts Institute of Technology, Cambridge, MA (US)

(21) Appl. No.: 17/602,558

(22) PCT Filed: Apr. 24, 2020
(86) PCT No.: PCT/US2020/029817

§ 371 Date: Oct. 8, 2021

Related U.S. Application Data
(60) Provisional application No. 62/838,564, filed on Apr. 25, 2019.

(51) Int. Cl. C08B 37/16; A61K 47/69; A61K 38/28; G01N 33/66

(52) U.S. Cl. CPC C08B 37/0012; C08B 37/0015; G01N 33/66; A61K 38/28; A61K 47/6951

(57) ABSTRACT

Provided herein are new cyclodextrin-based compounds, and pharmaceutically acceptable salts, stereoisomers, and isotopically labeled derivatives thereof, which are capable of binding sugars (e.g., glucose). The compounds provided herein may be conjugated to agents or tags (e.g., therapeutic agents such as insulin) to form conjugates. In another aspect, also provided herein are pharmaceutical compositions comprising the compounds and conjugates described herein. The compounds and conjugates described herein are capable of binding glucose; therefore, provided herein are methods of sensing or detecting glucose comprising contacting glucose with the compound or conjugate. In another aspect, also provided herein are methods of treating a disease (e.g., a metabolic disorder, such as diabetes) in a subject comprising administering to the subject a compound, conjugate, or composition provided herein.

### RELATED APPLICATIONS

This application claims priority under 35 U.S.C. § 119(e) to United States Provisional Patent Application, U.S.S.N. 62/838,564, filed Apr. 25, 2019, the entire contents of which in incorporated herein by reference.

### BACKGROUND OF THE INVENTION

Type 1 diabetes (T1D) is metabolic disorder caused by autoimmune destruction of pancreatic β-cells in the islets of Langerhans (see, e.g., Daneman, D. Type 1 diabetes. Lancet 367, 847-858 (2006). This condition results in absolute insulin deficiency and hyperglycemia (see, e.g., O'Sullivan, E. S., Vegas, A., Anderson, D. G. & Weir, G. C. Islets transplanted in immunoisolation devices: a review of the progress and the challenges that remain. Endocr. Rev. 32, 827-844 (2011); Chhabra, P. & Brayman, K. L. Current Status of Immunomodulatory and Cellular Therapies in Preclinical and Clinical Islet Transplantation. J. Transplant. 2011, e637692 (2011). Qi, M. Transplantation of Encapsulated Pancreatic Islets as a Treatment for Patients with Type 1 Diabetes Mellitus. Adv. Med. 2014, e429710 (2014)). There is no known cure for diabetes, and currently, 18 million people are affected worldwide. Insulin, a 51 amino-acid peptide produced by β-cells, regulates blood glucose levels by stimulating liver and muscle cells to metabolize glucose from the blood (see, e.g., Dabelea, D. The accelerating epidemic of childhood diabetes. Lancet 373, 1999-2000 (2009)), Persistent glycemic control is a key determinant in the long-term maintenance of diabetes (see, e.g., Pickup, J. C. Management of diabetes mellitus: is the pump mightier than the pen? Nature Rev. Endocrinol. 8, 425-433 (2012)). When left untreated, both prolonged hypoglycemia and hyperglycemia have proven to be life-threatening. Self-administration of exogenous insulin injections subcutaneously several times daily combined with close glucose monitoring is an important component in managing diabetes. Apart from being expensive, insulin injection therapies are painful and inconvenient with poor patient compliance. Several technologies have been developed to date to improve disease management in diabetes.

One of the clinically viable treatments involves the implantation of encapsulated islet cells obtained from xenogeneic or allogeneic sources. Although islet encapsulation restores insulin production, it is associated with adverse side effects and needs immunosuppressive medication see, e.g., Marzorati S, Melzi R, Citro A, Cantarelli E, Mercalli A, Scavini M et al. Engraftment versus immunosuppression: cost-benefit analysis of immunosuppression after intrahepatic murine islet transplantation. Transplantation 97, 1019-1026 (2014)). This has inspired research efforts toward the generation of a “fully synthetic pancreas” that will replicate the native insulin secreting function of a healthy pancreas maintaining normoglycemia (70-140 mg per dl) (see, e.g., American Diabetes Association. Standards of medical care in diabetes 2013. Diabetes Care 36 (Suppl. 1), 11-66 (2013)). Ideally, this system would be expected to quickly respond to elevated blood glucose levels releasing the appropriate amount of insulin and terminate release once normoglycemia is achieved without leaking insulin and maintaining basal insulin levels.

Development of synthetic glucose binding molecules that can recapitulate the function of a healthy pancreas toward the control of insulin secretion has emerged as a promising strategy for long-term control of type 1 diabetes (see, e.g., Bratlie, K. M.; York, R. L.; Invernale, M. A.; Langer R,; Anderson D. G. Materials for diabetes therapeutics. Adv. Healthc. Mater. 1, 267-284 (2012); Webber, M. J.; Anderson D. G. Smart approaches to glucose-responsive drug delivery. J Drug Target. 33, 651-655 (2015)). Glucose is a ubiquitous small-molecule analyte present in both healthy and diseased states in slightly different concentrations (see, e.g., Muggeo, M.; Zoppini, G.; Bonora, E.; Brun, E.; Bonadonna, R. C.; Moghetti, P.; Verlato, G. Fasting plasma glucose variability predicts 10-year survival of type 2 diabetic patients: the Verona Diabetes Study. Diabetes Care. 23, 45-50 (2000)). Hence, the major challenge in designing a glucose sensing material is refining the sensitivity of the device to detect a very small difference in glucose levels.

Compounds that bind glucose have important applications. For example, they can be conjugated to insulin to form glucose-responsive insulin insulin which becomes less active or inactive when glucose is not present. This may free diabetics from the fear of hypoglycemia, where glucose levels sink to relatively low levels. Over the last few decades, there are various glucose detection methods developed and combined with insulin formulation for glucose-responsive delivery (see, e.g., Sun. X.; James, T. D. Glucose Sensing in Supramolecular Chemistry. Chem. Rev. 115, 8001-8037 (2015); Bratlie, K. M.; York, R. L.; Invernale, M. A.; Langer R.; Anderson D. G Materials for diabetes therapeutics. Adv. Healthc. Mater. 1, 267-284 (2012); Webber, M. J.; Anderson D. G. Smart approaches to glucose-responsive drug delivery. J Drug Target. 33, 651-655 (2015)).

As another example, glucose-binding molecules can be used in continuous glucose monitors, which would allow diabetics to know their glucose levels at all times.

Previously, the use of phenylboronic acids (PBAs) to bind glucose for the generation of glucose-responsive materials and glucose-responsive insulin conjugates have been extensively explored. Work has established that the synthetic modification of insulin, with the aliphatic domain facilitating hydrophobic interactions and phenylboronic acid for glucose sensing, could afford both long-term and glucose-mediated insulin activity (see, e.g., Chou, D. H-C.; Webber, M. J.; Tanga, B. C.; Lin, A. B.; Thapa, L. S.; Deng, D.; Truong, J. V.; Cortinas, A. B.; Langer, R.; Anderson, G. Glucose-responsive insulin activity by covalent modification with aliphatic phenylboronic acid conjugates. Proc. Natl Acad. Sci. USA 112, 2401-2406 (2015)). In addition to PBA-based systems, elegant studies on tticarboxylic acids containing aromatic caged synthetic lectin showed very high glucose binding (see, e.g., Ke, C., Destecroix, H., Crump, M. P. & Davis, A. P. A simple and accessible synthetic lectin for glucose recognition and sensing. Nat Chem 4, 718-723 (2012); Das, G.; Hamilton, A. D. Carbohydrate recognition: Enantioselective sprirobifluroene diphosphonate receptors. Tetrahedron Lett. 38, 3675-3678 (1997)). In addition, previous work has demonstrated that phosphates and phosphonates along with carboxylic acids have potential utility in binding saccharides through covalent or hydrogen bond mediated interactions (see, e.g., Sun, X.; James, T. D. Glucose Sensing in Supramolecular Chemistry. Chem. Rev. 115, 8001-8037 (2015); Tromans, R. A.; Carter, T. S.; Chabanne, L.; Crump, M. P.; Li, H.; Matlock, J. V.; Orchard, M. G.; Davis, A. P. A biomimetic receptor for glucose. Nat Chem. 11, 52-56 (2019)). There is a need for further synthetic binders that show selectivity and sensitivity for glucose for use in modified responsive insulin.

### SUMMARY OF THE INVENTION

In one aspect, provided herein are new cyclodextrin-based compounds, and pharmaceutically acceptable salts, stereoisomers, and isotopically labeled derivatives thereof, which are capable of binding sugars (e.g., glucose). The cyclodextrin-based compounds provided herein comprise a cyclodextrin macrocycle, wherein two sugars of the cyclodextrin macrocycle are connected via a linker (also referred to as an “internal crosslink”). In certain embodiments, the linker (i.e., internal crosslink) connecting two sugars of the cyclodextrin macrocycle comprises one or more aromatic rings. Without wishing to be bound by any particular theory, incorporating one or more aromatic rings into the internal crosslink of the cyclodextrin-based compound induces hydrogen bonding and C—H π interactions between the compound and sugars (e.g., glucose). In certain embodiments, the compounds provided herein bind glucose and can be conjugated to therapeutic agents (e.g., insulin) and/or incorporated into glucose-sensing materials,

In certain embodiments, a compound provided herein is compound of Formula (1):

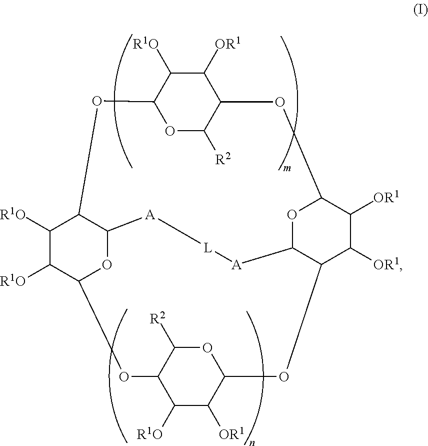

or a pharmaceutically acceptable salt, stereoisomer, or isotopically labeled derivative wherein L, A, R¹, R², m, and n are as defined herein,

As described herein, in certain embodiments, a compound provided here (e.g., a cyclodextrin-based compound) can be conjugated to an agent or tag (e.g., a therapeutic agent such as insulin). Therefore, provided herein are conjugates comprising a compound provided herein, or a pharmaceutically acceptable salt, stereoisomer, or isotopically labeled derivative thereof, conjugated to an agent or a tag. In certain embodiments, the compound is conjugated to an agent, and the agent is a therapeutic protein. In certain embodiments, the therapeutic protein is insulin (e.g., wild-type or modified insulin). In certain embodiments, the protein is wild-type insulin (i.e., native insulin). In certain embodiments, the protein is modified insulin (i.e., an insulin analog).

For example, in certain embodiments, a conjugate provided herein is of Formula (II):

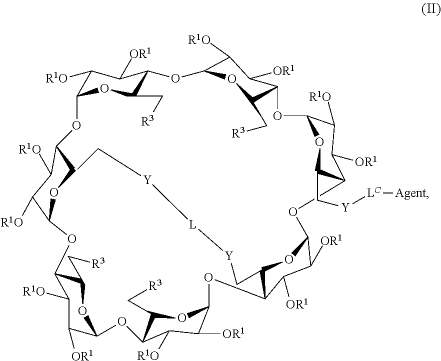

or a pharmaceutically acceptable salt, stereoisomer, or isotopically labeled derivative thereof, wherein R¹, R³, Y, L^{C}, and “Agent” are as defined herein.

In certain embodiments, a compound provided herein is conjugated to insulin to form a glucose-responsive form of insulin. In certain embodiments, the compound is conjugated to a glucose-sensing material. In certain embodiments, a compound provided herein is conjugated to a glucose-sensing material for use in glucose monitoring.

The present disclosure also provides pharmaceutical compositions comprising a compound or conjugate provided herein, or a pharmaceutically acceptable salt, stereoisomer, or isotopically labeled derivative thereof, and optionally a pharmaceutically acceptable excipient.

In another aspect, the compounds and compositions provided herein can be used to bind glucose. Binding glucose can be important step in sensing (e.g., detecting or quantifying) glucose in vivo or in vitro. Binding glucose can also be an important step in delivering therapeutic agents (e.g., insulin) in a glucose-responsive fashion. Therefore, provided herein is a method of sensing or detecting glucose comprising contacting the glucose with a compound or conjugate provided herein, or a pharmaceutically acceptable salt, stereoisomer, or isotopically labeled derivative thereof, or a pharmaceutical composition thereof.

In yet another aspect, also provided herein are methods for treating, diagnosing, or preventing a disease in a subject (e.g., a metabolic disorder, such as diabetes) comprising administering to the subject a compound or conjugate provided herein, or a pharmaceutically acceptable salt, stereoisomer, or isotopically labeled derivative thereof, or a pharmaceutical composition thereof.

Also encompassed by the disclosure are kits (e.g., pharmaceutical packs). The kits provided may comprise a pharmaceutical composition, compound, or conjugate described herein and a container (e.g., a vial, ampule, bottle, syringe, and/or dispenser package, or other suitable container).

The details of certain embodiments of the invention are set forth herein. Other features, objects, and advantages of the invention will be apparent from the Detailed Description, Figures, Examples, and Claims.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer, Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, New York, 1962); and Wilen, S. H., Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynl, and heterocyclic groups.

The term “alkyl” refers to a radical of a straight-chain or branched saturated hydrocarbon group having from 1 to 10 carbon atoms (“C₁₋₁₀ alkyl”). (“C₁₋₇ alkyl”). In some embodiments, an alkyl group has 1 to 6 carbon atoms (“C₁₋₆ alkyl”). Examples of C₁₋₆ alkyl groups include methyl (C₁), ethyl (C₂), propyl (C₃) (e.g., n-propyl, iso-propyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like.

The term “haloalkyl” is a substituted alkyl group, wherein one or more of the hydrogen atoms are independently replaced by a halogen, e.g., fluoro, bromo, chloro, or iodo.

The term “heteroalkyl” refers to an alkyl group, which further includes at least one heteroatom (e.g., 1, 2, 3, or 4 heteroatoms) selected from oxygen, nitrogen, or sulthr within (i.e., inserted between adjacent carbon atoms of) and/or placed at one or more terminal position(s) of the parent chain.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 4 carbon atoms (“C₂₋₄ alkenyl”). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In an alkenyl group, a C═C double bond for which the stereochemistry is not specified (e.g., —CH═CHCH₃ or

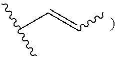

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain.

The term “alkynyl” refers to a radical of a straight-chain or branched hydrocarbon group having from 2 to 10 carbon atoms and one or more carbon-carbon triple bonds (e.g., 2, 3, or 4 triple bonds) (“C₂₋₁₀ alkynyl”). In some embodiments, an alkynyl group has 2 to 4 carbon atoms (“C₂₋₄ alkynyl”). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 6 ring carbon atoms (“C₃₋₆ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. As the foregoing examples illustrate, in certain embodiments, the carbocyclyl group is either monocyclic (“monocyclic carbocyclyl”) or polycyclic (e.g., containing a fused, bridged or Spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spino ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted arvi”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n±2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryllheteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

in some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The tern) “optionally substituted” refers to being substituted or unsubstituted. In certain embodiments, alkyl, alkenyl alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —C(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, −OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein Y is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂Rₐₐ, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, 6-14 aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₃₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2,1, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₁₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —N₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —C(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), SO₂NH₂, SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, —C(═S)NH(C₁₋₆ alkyl), C(═S)NH₂, C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, substituents include: halogen, —CN, —NO₂, —N₃, —SO₂H, —SO₃H, —OH, —OC₁₋₆ alkyl, —ON(C₁₋₆ alkyl)₂, —N(C₁₋₆ alkyl)₃⁺X⁻, —NH(C₁₋₆ alkyl )₂⁺X⁻, —NH₂(C₁₋₆ alkyl)⁺X⁻, —NH₃⁺X⁻, —N(OC₁₋₆ alkyl)(C₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(—NH)N(C₁₋₆ alkyl)₂, —OC(═NH)NH(C₁₋₆ alkyl), —OC(═NH)NH₂, —NHC(═NH)N(C₁₋₆ alkyl₂, —NHC(═NH)NH₂, —NHSO₂(C₁₋₆ alkyl), —SO₂N(C₁₋₆ alkyl), —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(—S)N(C₁₋₆ alkyl)₂, C(═S)NH(C₁₋₆ alkyl), C(═S)NH₂, —C(═O)S(C₁₋₆ alkyl), —C(—S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal hydrogens can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(O)R^{aa}, —OCO₂R^{aa}, —OC(O)N(R^{bb})R^{aa}, —OC(═NR^{bb})R^{aa}, —OC(—NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb}, and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “tri substituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{x1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroaryithioxy, mono- or di-aliphaticamino, mono- or di-heteroaliphaticamino, mono- or di-alkylamino, mono- or di-heteroalkylamino, mono- or di-arylamino, or mono- or di-heteroarvlamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arviamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not he further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂R^{aa}, —C(═O)SR^{aa}, —C(═S)SR^{aa}), amides (e.g., —C(═O)N(R^{bb})₂, —C(═O)NR^{bb}SO₂R^{aa}, —C(═S)N(R^{bb})₂), and imines (e.g., —C(═NR^{bb})R^{aa}, —C(NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can he substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, 6-14 aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa}, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trilluorotnethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fuinarate, maleate, malate, malonate, gluconate, succinate, glutarate, adipate, pimelate, suberate, azelate, sebacate, salicylate, phthalates, aspartate, glutamate, and the like), and carboranes.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

### Other Definitions

The following definitions are more general terms used throughout the present application.

As generally defined herein, “cyclodextrin” refers to a macrocyclic polymer comprising 4 or more sugar monomers (i.e., subunits) linked by glycosidic bonds (often referred to as “cyclic oligosaccharide”). In some instances, a cyclodextrin comprises 4 or more glucose subunits. In some instances, a cyclodextrin comprises 4 or more glucose subunits linked by 1,4-glycosidic bonds. In certain embodiments, a cyclodextrin is of the following formula:

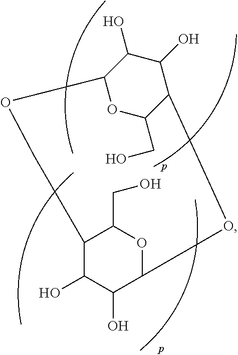

wherein each instance of p is independently an integer from 2 to 16, inclusive. In certain embodiments, a “cyclodextrin” comprises 5 to 8 glucose subunits linked by linked by 1,4-glycosidic bonds. Examples of cyclodextrins include, but are not limited to, α-cyclodextrin (6 glucose subunits) (α-CD), β-cyclodextrin (7 glucose subunits) (β-CD), and γ-cyclodextrin (8 glucose subunits) (-γ-CD), each of which are shown below.

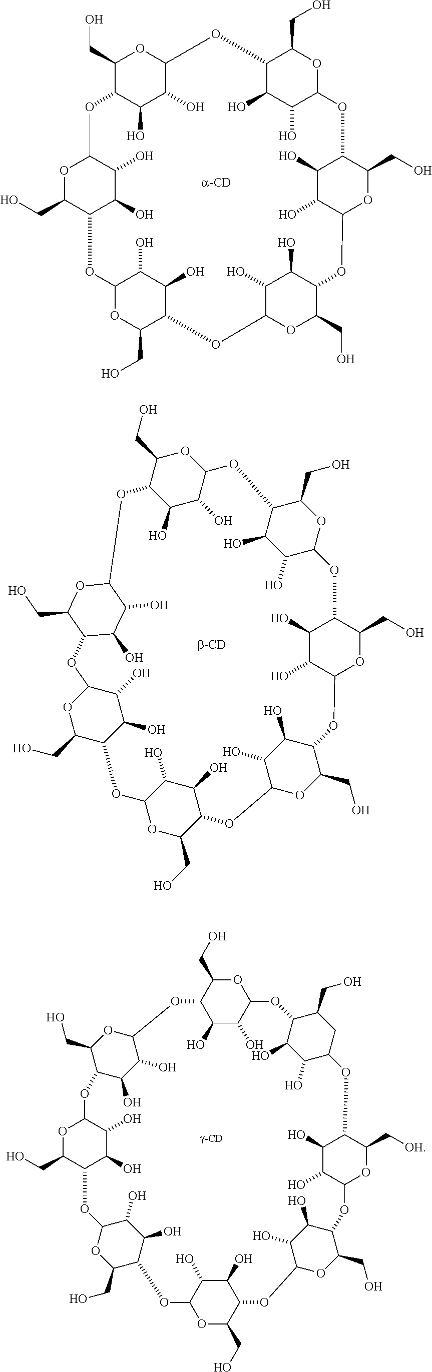

The term “sugar” refers to monosaccharides, disaccharides, or polysaccharides. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose d-glucose, for example, has the formula C₆H₁₂O₆, of which all but two of its six carbons atoms are stereogenic, making d-glucose one of the 16 (i.e., 2⁴) possible stereoisomers. The assignment of d or l is made according to the orientation of the asymmetric carbon furthest from the carbonyl group: in a standard Fischer projection if the hydroxyl group is on the right the molecule is a d sugar, otherwise it is an l sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary di saccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose.

As used herein, the term “polymer” refers to any substance comprising at least two repeating structural units (i.e., “monomers”) which are associated with one another. In some embodiments, monomers are covalently associated with one another. Polymers may be homopolymers or copolymers comprising two or more monomers. In terms of sequence, copolymers may be random, block, graft, or comprise a combination of random, block, and/or graft sequences. In some embodiments, block copolymers are diblock copolymers. In some embodiments, block copolymers are triblock copolymers. In some embodiments, polymers can be linear or branched polymers.

A “protein,” “peptide,” or “polypeptide” comprises a polymer of amino acid residues linked together by peptide bonds. The term refers to proteins, polypeptides, and peptides of any size, structure, or fimction. Typically, a protein will be at least three amino acids long. A protein may refer to an individual protein or a collection of proteins. Proteins preferably contain natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical agent such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these. A “native” or “wild type” protein or peptide refers to the protein or peptide as it is found in nature (i.e., without further modification). In certain embodiments, a protein useful in the present invention is a therapeutic protein. Examples of therapeutic proteins are provided below and elsewhere herein.

As generally defined herein, “therapeutic protein” refers to any protein or protein-based therapy that may be administered to a subject and have a therapeutic effect. Such therapies include protein replacement and protein supplementation therapies. Such therapies also include the administration of exogenous or foreign protein, antibody therapies, and cell or cell-based therapies. Therapeutic proteins include infusible therapeutic proteins, enzymes, enzyme cofactors, hormones, blood clotting factors, cytokines, growth factors, monoclonal antibodies, and polyclonal antibodies. In certain embodiments, a therapeutic protein is insulin.

As used herein, the terms “conjugated” when used with respect to two or more moieties, means that the moieties are physically associated or connected with one another, either directly or via one or more additional moieties that serve as a linking agent, to form a structure that is sufficiently stable so that the moieties remain physically associated under the conditions in which the structure is used, e.g., physiological conditions,

The term “amino acid” refers to a molecule containing both an amino group and a carboxyl group. Amino acids include alphaamino acids and betaamino acids, the structures of which are depicted below. In certain embodiments, an amino acid is an alpha amino acid.

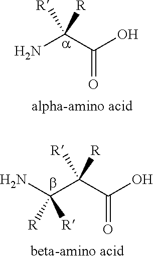

Suitable amino acids include, without limitation, natural alphaamino acids such as D- and L-isomers of the 20 common naturally occurring alphaamino acids found in peptides (e.g., A, R, N, C, D, Q, E, G, H, I, L, K, M, F, P, S, T, W, Y, V, as provided below), unnatural alpha-amino acids natural beta-amino acids (e.g., beta-alanine), and unnnatural beta-amino acids. Exemplary natural alpha-amino acids include L-alanine (A), L-arginine (R), L-asparagine (N), L-aspartic acid (D), L-cysteine (C), L-glutamic acid (E), L-glutamine (Q), glycine (G), L-histidine (H), L-isoleucine L-leucine (L), L-lysine (K), L-methionine (M), L-phenylalanine (F), L-proline (P), L-serine (S), L-threonine (T), L-tryptophan (W), L-tyrosine (Y), and L-valine (V). Exemplary unnatural alpha-amino acids include D-arginine, D-asparagine, D-aspartic acid, D-cysteine, D-glutamic acid, D-glutamine, D-histidine, D-isoleucine, D-leucine, D-lysine, D-methionine, D-phenylalanine, D-proline, D-serine, D-threonine, D-tryptophan, D-tyrosine, D-valine, Di-vinyl, α-methyl-alanine (Aib), α-methyl-arginine, α-methyl-asparagine, α-methyl-aspartic acid, α-methyl-cysteine, α-methyl-glutamic acid, α-methyl-glutamine, α-methyl-histidine, α-methyl-isoleucine, α-methyl-leucine, α-methyl-lysine, α-methyl-methionine, α-methyl-phenylalanine, α-methyl-proline, α-methyl-senne, α-methyl-threonine, α-methyl-tryptophan, α-methyl-tyrosine, α-methyl-valine, norleucine, terminally unsaturated alphaamino acids and bis alpha-amino acids (e.g., modified cysteine, modified lysine, modified tryptophan, modified serine, modified threonine, modified proline, modified hi stidine, modified alanine, and the like). There are many known unnatural amino acids any of which may be included in the peptides of the present invention. See for example, S. Hunt, The Non-Protein Amino Acids: In Chemistry and Biochemistry of the Amino Acids, edited by G. C. Barrett, Chapman and Hall, 1985.

As used herein, the term “salt” refers to any and all salts, and encompasses pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Compounds described herein are also provided, and can be administered, as a free base.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed, in other embodiments, treatment may be administered in the absence of signs or symptoms of the disease. For example, treatment may be administered to a susceptible subject prior to the onset of symptoms. Treatment may also be continued after symptoms have resolved, for example, to delay or prevent recurrence.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. Alternatively, in a separate method or use, the invention may be used, where indicated and effective, as a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating in any disease or condition described. In certain embodiments, a therapeutically effective amount is an amount sufficient for binding glucose. In certain embodiments, a therapeutically effective amount is an amount sufficient for treating diabetes in a subject. In certain embodiments, a therapeutically effective amount is an amount sufficient for binding glucose and treating diabetes in a subject.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding glucose. In certain embodiments, a prophylactically effective amount is an amount sufficient for preventing diabetes. In certain embodiments, a prophylactically effective amount is an amount sufficient for binding glucose and preventing diabetes.

The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinetnia, insulin resistance, and obesity.

A “diabetic condition” refers to diabetes and pre-diabetes. “Diabetes” refers to a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger). There are several types of diabetes. Type I diabetes results from the body's failure to produce insulin, and presently requires the person to inject insulin or wear an insulin pump. Type II diabetes results from insulin resistance a condition in which cells fail to use insulin properly, sometimes combined with an absolute insulin deficiency. Gestational diabetes occurs when pregnant women without a previous diagnosis of diabetes develop a high blood glucose level. Other forms of diabetes include congenital diabetes, which is due to genetic defects of insulin secretion, cystic fibrosis-related diabetes, steroid diabetes induced by high doses of glucocorticoids, and several forms of monogenic diabetes, e,g., mature onset diabetes of the young (e.g., MODY 1, 2, 3, 4, 5, 6, 7, 8, 9, or 10). Pre-diabetes indicates a condition that occurs when a person's blood glucose levels are higher than normal but not high enough for a diagnosis of diabetes. All forms of diabetes increase the risk of long-term complications. These typically develop after many years, but may be the first symptom in those who have otherwise not received a diagnosis before that time. The major long-term complications relate to damage to blood vessels. Diabetes doubles the risk of cardiovascular disease and. macrovascular diseases such as ischemic heart disease (angina, myocardial infarction), stroke, and peripheral vascular disease. Diabetes also causes microvascular complications, e.g., damage to the small blood vessels. Diabetic retinopathy, which affects blood vessel formation in the retina of the eye, can lead to visual symptoms, reduced vision, and potentially blindness. Diabetic nephropathy, the impact of diabetes on the kidneys, can lead to scarring changes in the kidney tissue, loss of small or progressively larger amounts of protein in the urine, and eventually chronic kidney disease requiring dialysis. Diabetic neuropathy is the impact of diabetes on the nervous system, most commonly causing numbness, tingling and pain in the feet and also increasing the risk of skin damage due to altered sensation. Together with vascular disease in the legs, neuropathy contributes to the risk of diabetes-related foot problems, e.g., diabetic foot ulcers, that can be difficult to treat and occasionally require amputation,

### BRIEF DESCRIPTION OF THE DRAWINGS

The accompanying drawings, which constitute a part of this specification, illustrate several embodiments of the invention and together with the description, serve to explain the principles of the invention.

FIGS. 1A-1B. Examples of cyclodextrin caged structures (CDCs). FIG. 1A. CDC1-CDC3, having glucose stable in the binding pocket for more than 25 nanoseconds. FIG. 1B. CDC1 displaying receptor-glucose complexation over 100 nanoseconds in molecular dynamics (MD) simulation.

FIG. 2A. Synthesis of CDC1.

FIG. 2B. Binding isotherm for CDC1.

FIGS. 3A-3C. Additional examples of cyclodextrin caged structures. FIG. 3A. CDC4-CDC7 having glucose stable in the binding pocket for more than 25 nanoseconds. CDC7 stabilizes glucose in the pocket for more than 100 nanoseconds. FIG. 3B. Cyclodextrin caged structures CDC8-CDC11 having glucose stable in the binding pocket for more than 100 nanoseconds. FIG. 3C. CDC11 displaying receptorglucose complexation over 100 nanoseconds in MD simulation.

FIG. 4A. Synthesis of CDC4-CDC7.

FIG. 4B. Synthesis of CDC11.

FIG. 4C. Alternative synthesis of CDC11.

FIGS. 5A-5C outline strategies for the preparation of smart insulin. FIG. 5A. Preparation of glycosylated insulin-CDC conjugate by utilizing non-covalent interactions between binder with glycosylated insulin. FIG. 5B. Preparation of glycosylated insulin-CDC polymer conjugate and insulin release in the presence of glucose. FIG. 5C. Preparation of insulin CDC-adamantane hydrogel and insulin release in the presence of glucose.

### DETAILED DESCRIPTION OF CERTAIN EMBODIMENTS

Provided herein are new cyclodextrin-based compounds, and pharmaceutically acceptable salts, stereoisomers, and isotopically labeled derivatives thereof, which are capable of binding sugars (e.g., glucose). The compounds provided herein may be conjugated to agents or tags (e.g., therapeutic agents, such as insulin) to form conjugates. In another aspect, also provided herein are pharmaceutical compositions comprising the compounds and/or conjugates described herein. The compounds and conjugates described herein are capable of binding glucose; therefore, provided herein are methods of sensing or detecting glucose comprising contacting glucose with the compound or conjugate. In another aspect, also provided herein are methods of treating a disease (e.g., a metabolic disorder such as diabetes) in a subject comprising administering to the subject a compound, conjugate, or composition provided herein.

### Cyclodextrin-Based Compounds

In one aspect, provided herein are new cyclodextrin-based compounds, and pharmaceutically acceptable salts, stereoisomers, and isotopically labeled derivatives thereof, which are capable of a binding sugar (e.g., glucose). The cyclodextrin-based compounds provided herein comprise a cyclodextrin macrocycle, wherein two sugars of the cyclodextrin macrocycle are connected via a linker (also referred to as an “internal crosslink”). In certain embodiments, the linker (i.e., internal crosslink) connecting the two sugars of the cyclodextrin macrocycle comprises one or more aromatic rings. Without wishing to be bound by a particular theory, incorporating one or more aromatic rings into the internal crosslink of the cyclodextrin-based compound induces hydrogen bonding and C—H π interactions between the compound and sugars (e.g., glucose). In certain embodiments, the compounds provided herein bind glucose and can be conjugated to therapeutic agents (e.g., insulin) and/or incorporated into glucose-sensing materials.

Provided herein are compounds, and pharmaceutically acceptable salts, stereoisomers, and isotopically labeled derivatives thereof, comprising a cyclodextrin, wherein two sugars of the cyclodextrin are connected via a linker; and the linker comprises one or more aromatic rings.

The cyclodextrin can be any cyclodextrin as defined herein. In certain embodiments, the cyclodextrin is an α-cyclodextrin, β-cyclodextrin, or γ-cyclodextrin. In certain embodiments, the cyclodextrin is an α-cyclodextrin. In certain embodiments, the cyclodextrin is a β-cyclodextrin. In certain embodiments, the cyclodextrin is a γ-cyclodextrin.

The compounds described herein comprise a cyclodextrin, wherein two sugars of the cyclodextrin macrocycle are connected via a linker (i.e., internal crosslink). In certain embodiments, the sugars connected via a linker are separated by 1, 2, or 3 sugars in the cyclodextrin macrocycle. In certain embodiments, the sugars connected via a linker are separated by 1 sugar, In certain embodiments, the sugars connected via a linker are separated by 2 sugars. In certain embodiments, the sugars connected via a linker are separated by 3 sugars. The two sugars connected via a liker can connected through any positions on the sugars. In certain embodiments, the linker comprises 1-200 non-hydrogen atoms total. In certain embodiments, the linker comprises 1-100 non-hydrogen atoms total. In certain embodiments, the linker comprises 1-50 non-hydrogen atoms total. In certain embodiments, the linker comprises 1-20 non-hydrogen atoms total.

As described herein, in certain embodiments, the linker connecting the two sugars of the cyclodextrin macrocycle comprises one or more aromatic rings. In certain embodiments, the one or more aromatic rings are selected from the group consisting of optionally substituted aryl, optionally substituted arylene, optionally substituted heteroaryl, and optionally substituted heteroarylene. The one or more aromatic rings may be monocyclic, bicyclic, tricyclic, or polycyclic. In other embodiments, the one or more aromatic rings are selected from those included in the definition of “L” provided herein.

In certain embodiments, the linker (i.e., internal crosslink) connecting the two sugars of the cyclodextrin is of the formula:

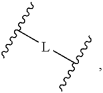

wherein:

L is optionally substituted alkylene, optionally substituted heteroalkylene, optionally substituted alkenylene, optionally substituted alkynylene, optionally substituted carbocyclylene, optionally substituted heterocyclylene, optionally substituted aryier optionally substituted heteroarylene, optionally substituted acylene, —N(R^{N})—, —O—, —S—, —C(R^{C})₂, —C(═O)—, —C(═O)N(R^{N})—, —N(R^{N})C(═O)—, —N(R^{N})C(═O)N(R^{N})—, —C(═O)O—, —OC(═O)—, —OC(═O)O—, —OC(═O)N(R^{N})—, —N(R^{N})C(═O)O—, or any combination thereof;

provided that L comprises one or more moieties selected from the group consisting of optionally substituted aryl, optionally substituted arylene, optionally substituted heteroaryl, optionally substituted heteroarylene, optionally substituted arylalkyl, and optionally substituted heteroarylalkyl; and

each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or wherein two R^{N} groups attached to the same nitrogen atom are taken together with the intervening atoms to form optionally substituted heterocyclyl or optionally substituted heteroaryl; and

each instance of R^{C} is independently hydrogen, halogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —CN, —OR^{O}, —N(R^{N})₂, or —SR^{S}; or two R^{C} are taken together to form an oxo group.

In certain embodiments, the linker (i.e., internal crosslink) connecting the two sugars of the cyclodextrin is of the formula:

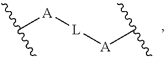

L is optionally substituted alkylene, optionally substituted heteroalkylene, optionally substituted alkenylene, optionally substituted alkynylene, optionally substituted carbocyclylene, optionally substituted heterocyclylene, optionally substituted arylene, optionally substituted heteroaryiene, optionally substituted acylene, —N(R^{N})—, —O—, —S—, —C(R^{C})₂—, —C(═O)—, —C(═O)N(R^{N})—, —N(R^{N})C(═O)—, —N(R^{N})C(═O)N(R^{N})—, —C(═O)O—, —C(═O)—, —OC(═O)O—, —OC(═O)N(R^{N})—, —N(R^{N})C(═O)O—, or any combination thereof;

provided that L comprises one or more moieties selected from the group consisting of optionally substituted aryl, optionally substituted arylene, optionally substituted heteroaryl, optionally substituted heteroarylene, optionally substituted aryialkyl, and optionally substituted heteroarylalkyl; and

each instance of A is independently a bond, optionally substituted alkylene, optionally substituted heteroalkylene, —O—, —N(R^{N})—, —C(R^{C})₂—, —C(═O)—, —C(R^{C})₂O—, —C(R^{C})₂N(R^{N})—, or —C(R^{C})₂S—;

each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or wherein two R^{N} groups attached to the same nitrogen atom are taken together with the intervening atoms to form optionally substituted heterocyclyl or optionally substituted heteroaryl; and

each instance of R^{C} is independently hydrogen, halogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —CN, —OR^{O}, —N(R^{N})₂, —SR^{S}; or two R^{C} are taken together to form an oxo group.

In addition to two sugars of the cyclodextrin being connected via a linker, the cyclodextrin may comprise one or more additional modifications. For example, one or more positions on the sugars of the cyclodextrin may be substituted (e.g., one or more carbons are substituted, or one or more —OH groups are substituted or protected). As another example, one or more groups on the sugars may be removed or replaced (e.g., one or more —OH groups are replaced with amino or thiol moieties). In certain embodiments, one or more groups on the sugars may be removed or replaced with an electrophile or a moiety for conjugation (e.g., a click chemistry handle). In other embodiments, one or more sugars of the cyclodextrin macrocycle may be replaced by different sugars. Several non-limiting examples of cyclodextrins comprising an internal crosslink, as well as one or more additional modifications, are provided herein.

In certain embodiments, a compound provided herein is compound of Formula (1):

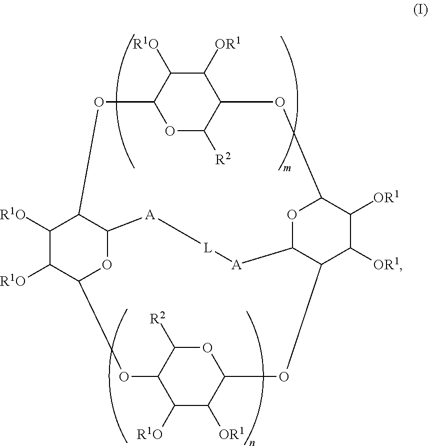

or a pharmaceutically acceptable salt, stereoisomer, or isotopically labeled derivative wherein:

m is an integer between 1 and 10, inclusive;

n is an integer between 1 and 10, inclusive;

L is a linker comprising optionally substituted alkylene, optionally substituted heteroalkylene, optionally substituted alkenylene, optionally substituted alkynylene, optionally substituted carbocyclylene, optionally substituted heterocyclylene, optionally substituted arylene, optionally substituted heteroarylene, optionally substituted acylene, —N(R^{N})—, —O—, —S—, —C(R^{C})₂—, —C(═O)—, —C(═O)N(R^{N})—, —N(R^{N})C(═O)—, —N(R^{N})C(═O)N(R^{N})—, —C(═O)O—, —OC(═O)—, —OC(═O)O—, —OC(═O)N(R^{N})—, —N(R^{N})C(═O)O—, or any combination thereof;

provided that L comprises one or more moieties selected from the group consisting of optionally substituted aryl, optionally substituted arylene, optionally substituted heteroaryl, optionally substituted heteroarylene, optionally substituted arylalkyl, and optionally substituted heteroarylalkyl;

each instance of A is independently a bond, optionally substituted alkylene, optionally substituted heteroalkylene, —O—, —N(R^{N})—, —S—, —C(R^{C})₂—, —C(═O)—, —C(R^{C})₂O—, —C(R^{C})₂N(R^{N})—, or —C(R^{C})₂S—;

each instance of R¹ is independently hydrogen, optionally substituted alkyl, optionally substituted carbocyclyl, optionally substituted heterocyclyl, optionally substituted aryl, optionally substituted heteroaryl, optionally substituted optionally substituted acyl, an oxygen protecting group, or a sugar;

each instance of R² is independently optionally substituted alkyl, optionally substituted heteroalkyl, optionally substituted carbocyclyl, optionally substituted heterocyclyl, optionally substituted aryl, optionally substituted heteroaryl, optionally substituted acyl, —OR^{O}—, —N(R^{N})₂, —SR^{S}, —C(R^{C})₂OR^{O}, —C(R^{C})₂N(R^{N})₂, —C(R^{C})₂SR^{S}, or a sugar;

each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;

each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or wherein two R^{N} groups attached to the same nitrogen atom are taken together with the intervening atoms to form optionally substituted heterocyclyl or optionally substituted heteroaryl;

each instance of R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a sulfur protecting group; and

each instance of R^{C} is independently hydrogen, halogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —CN, —OR^{O}, —N(R^{N})₂, —SR^{S}; or two R^{C} are taken together to form an oxo group.

In certain embodiments, compound of Formula (I) is a compound of Formula (I-a):

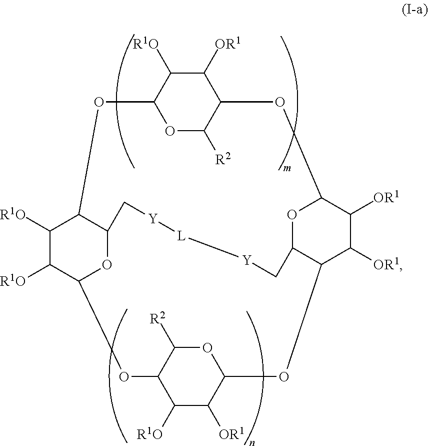

or a pharmaceutically acceptable salt, stereoisomer, or isotopically labeled derivative thereof, wherein:

each instance of Y is independently a bond, —C(R^{C})₂, —O—, —S—, or —N(R^{N})—.

In certain embodiments, a compound of Formula (I) is a compound of Formula (I-b):

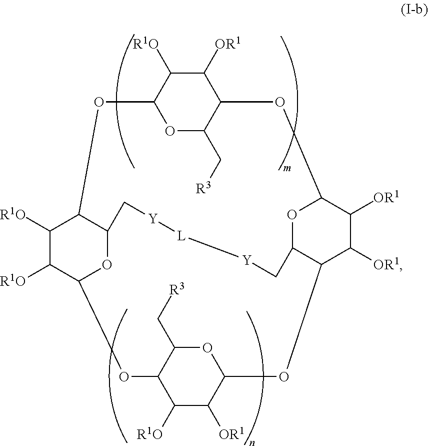

or a pharmaceutically acceptable salt, stereoisomer, or isotopically labeled derivative thereof, wherein:

each instance of R³ is independently —OR^{O}, —N(R^{N})₂, or —SR^{S}.

In certain embodiments, a compound of Formula (I) is a compound of Formula (I-c):

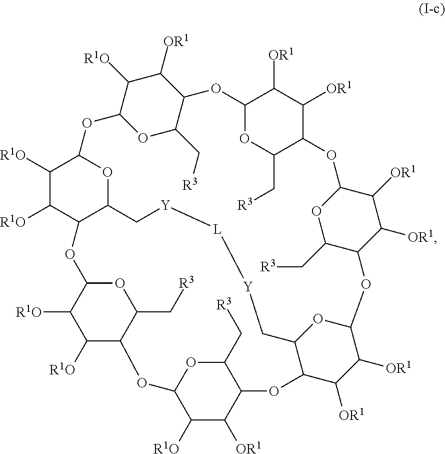

or a pharmaceutically acceptable salt, stereoisomer, or isotopically labeled derivative thereof.

In certain embodiments, a compound of Formula (I) is a compound of Formula (I-d):

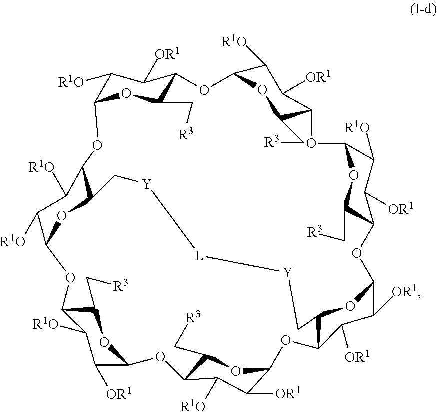

or a pharmaceutically acceptablesalt, stereoisomer, or isotopically labeled derivative thereof.

In certain embodiments, a compound of Formula (I) is a compound of Formula (I-e):

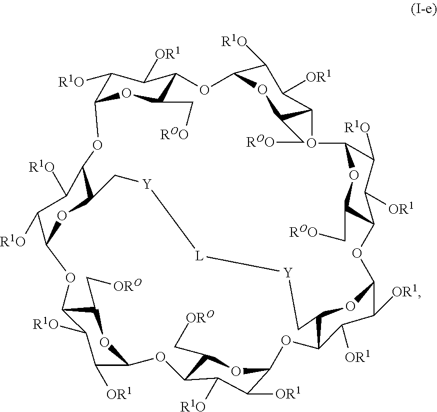

or a pharmaceutically acceptable salt, stereoisomer, or isotopically labeled derivative thereof.

In certain embodiments, compound of Formula (I) is a compound of Formula (I-f):

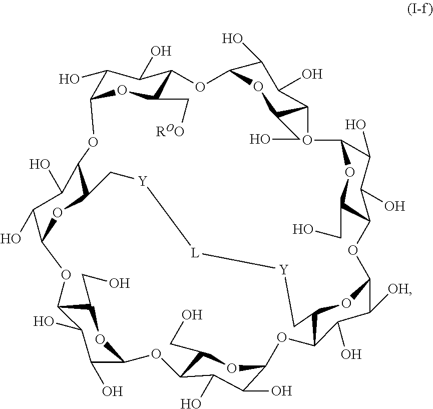

or a pharmaceutically acceptable salt, stereoisomer, or isotopically labeled derivative thereof.

In certain embodiments, a compound of Formula (I) is a compound of Formula (I-g):

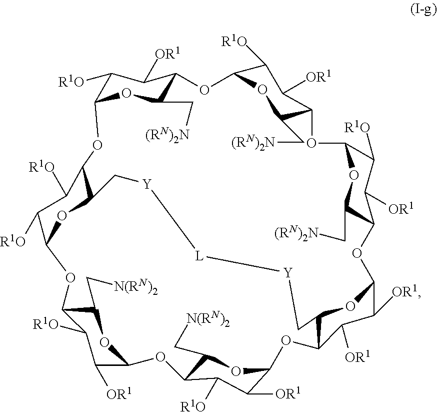

or a pharmaceutically acceptable salt, stereoisomer, or isotopically labeled derivative thereof.

In certain embodiments, compound of Formula (I) is a compound of Formula (I-h):

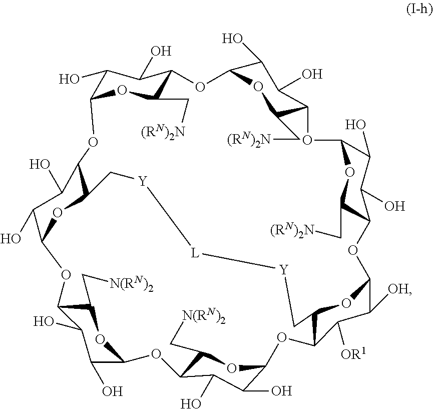

or a pharmaceutically acceptable salt thereof.

In certain embodiments, a compound provided herein is selected from the group consisting of:

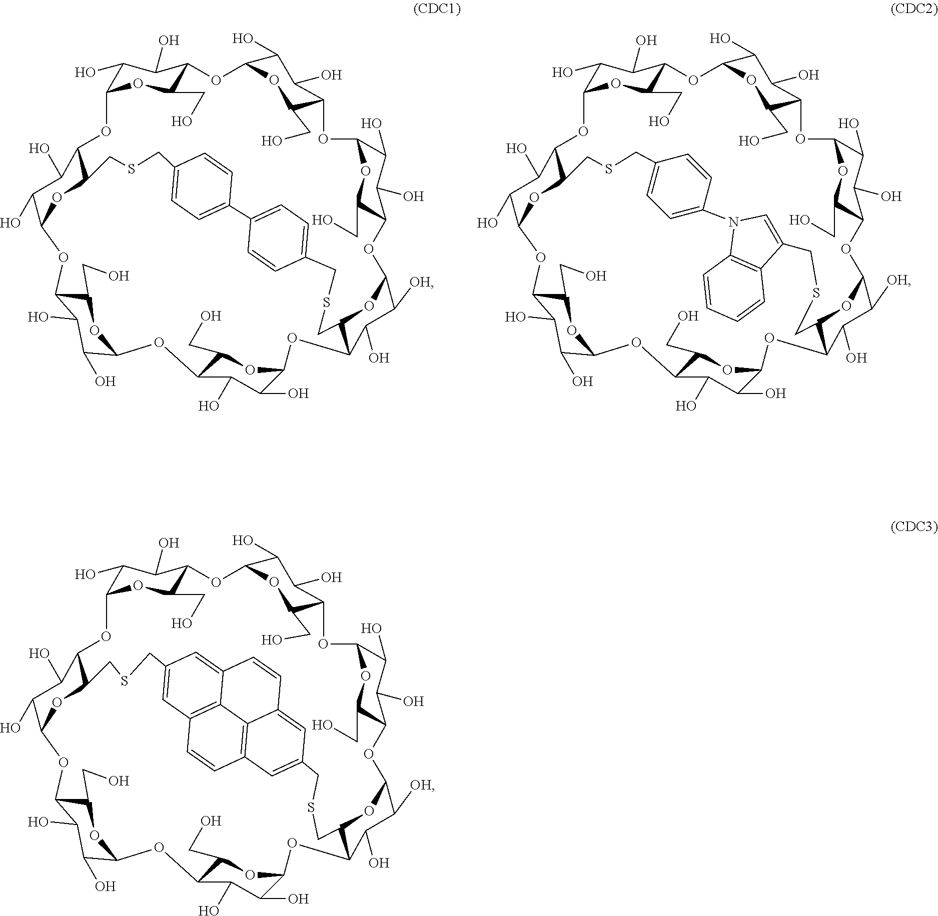

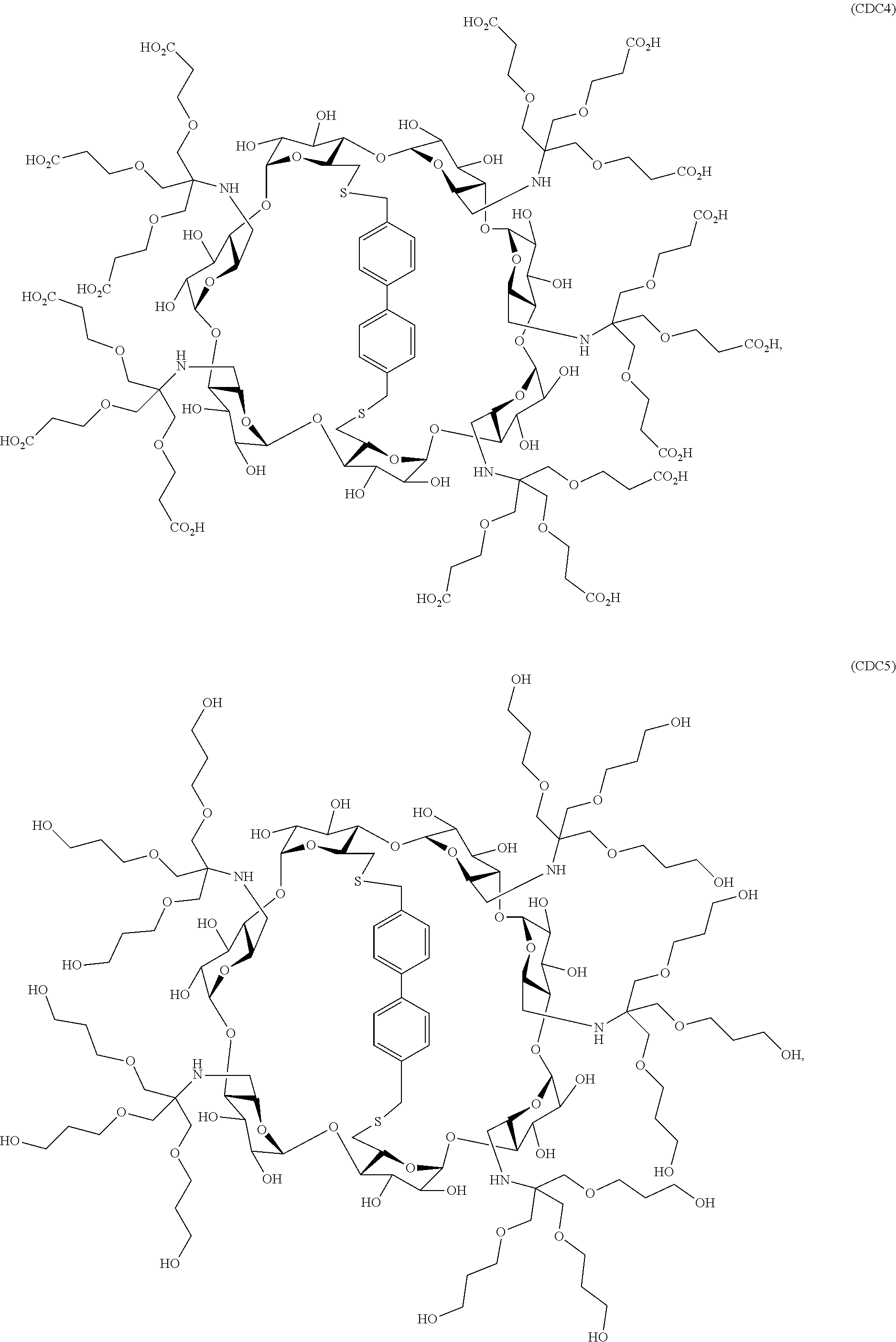

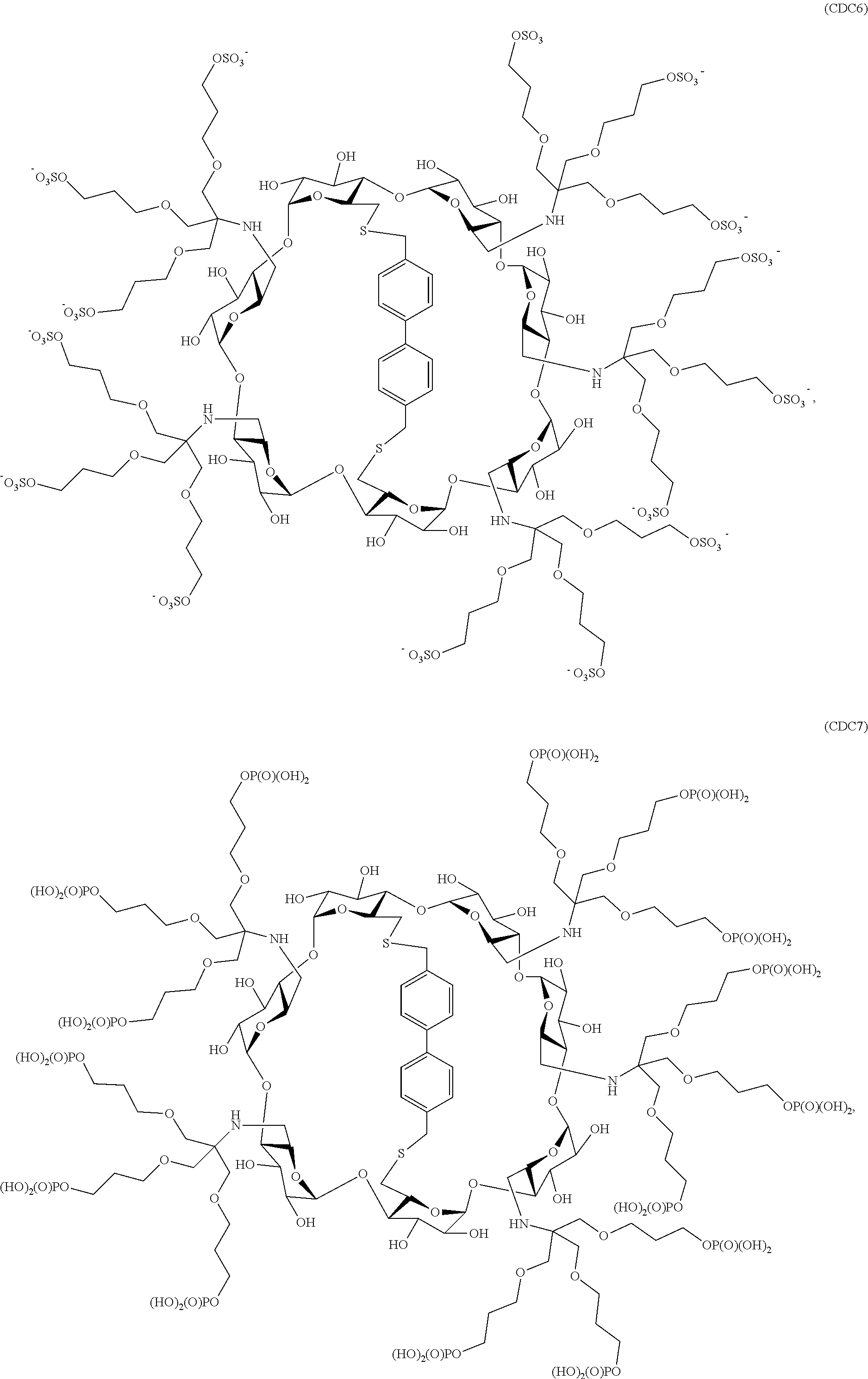

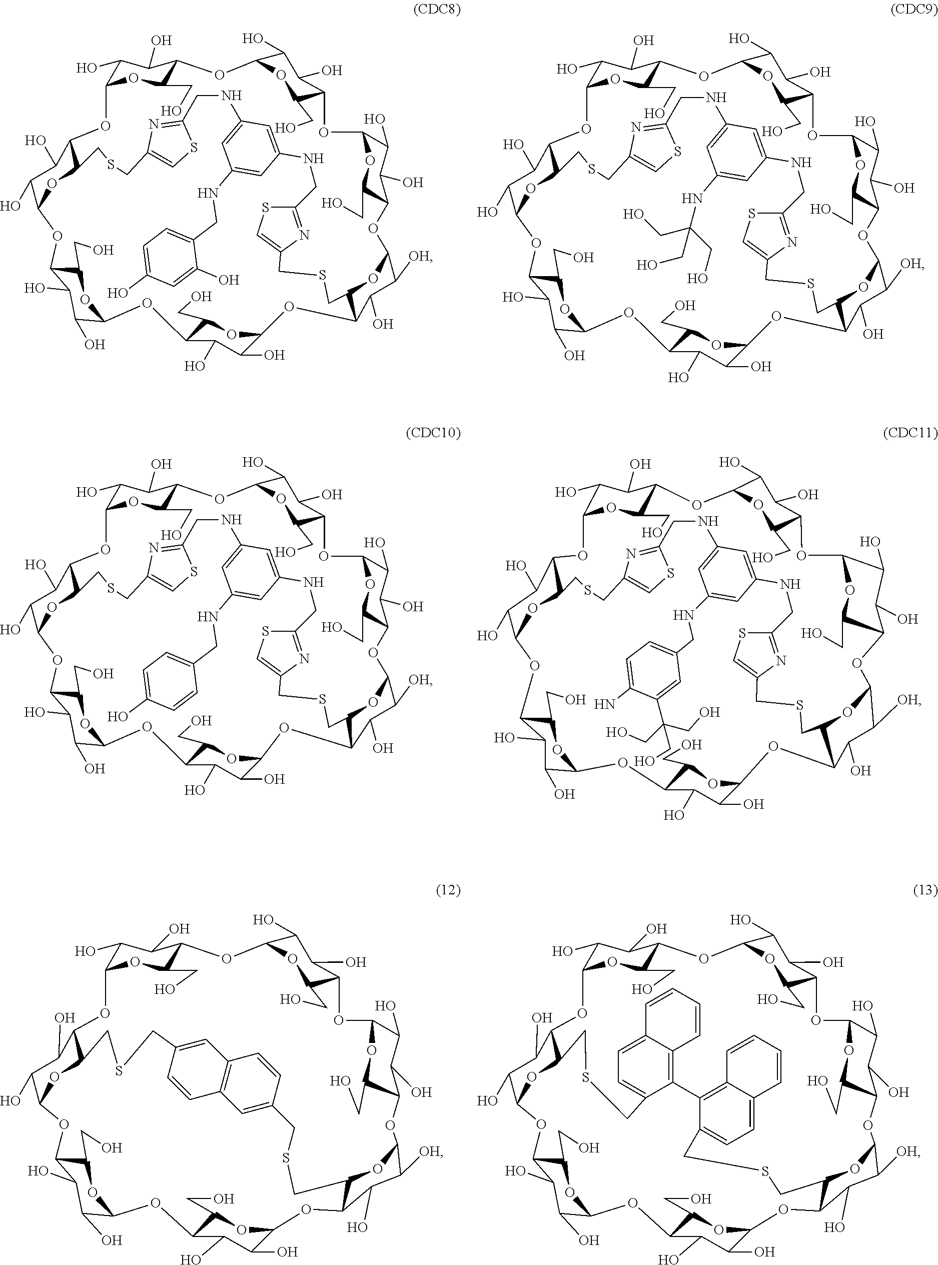

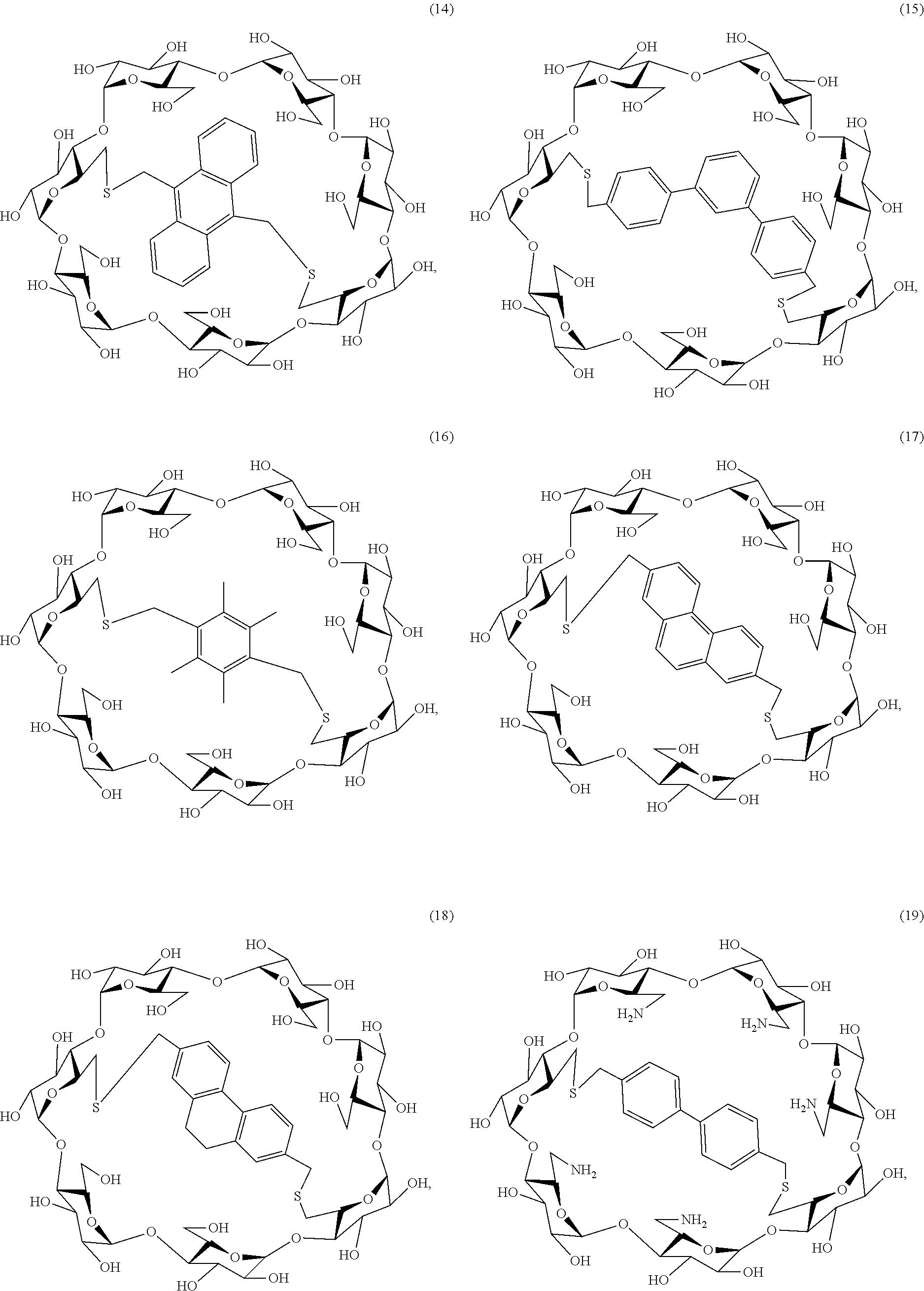

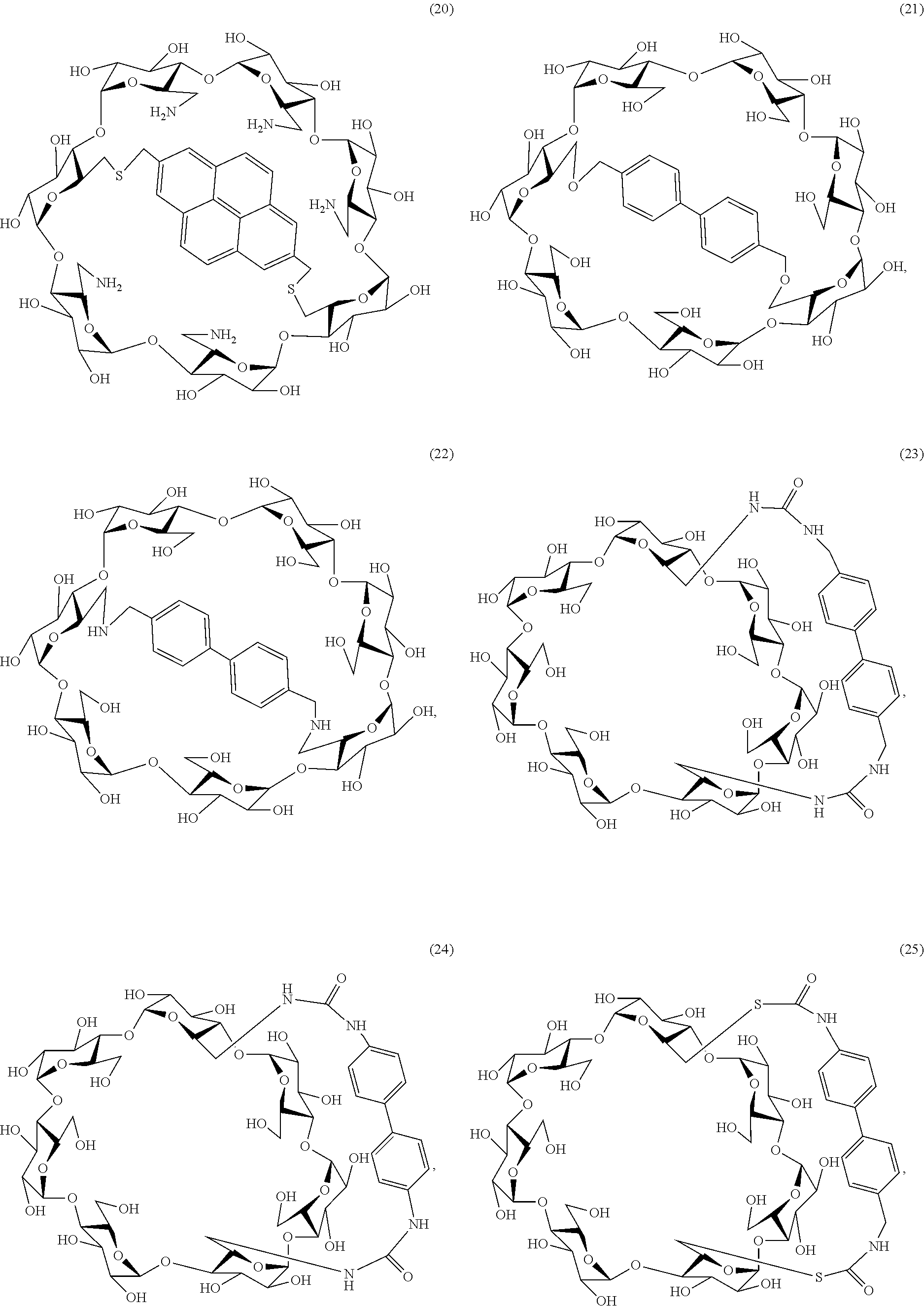

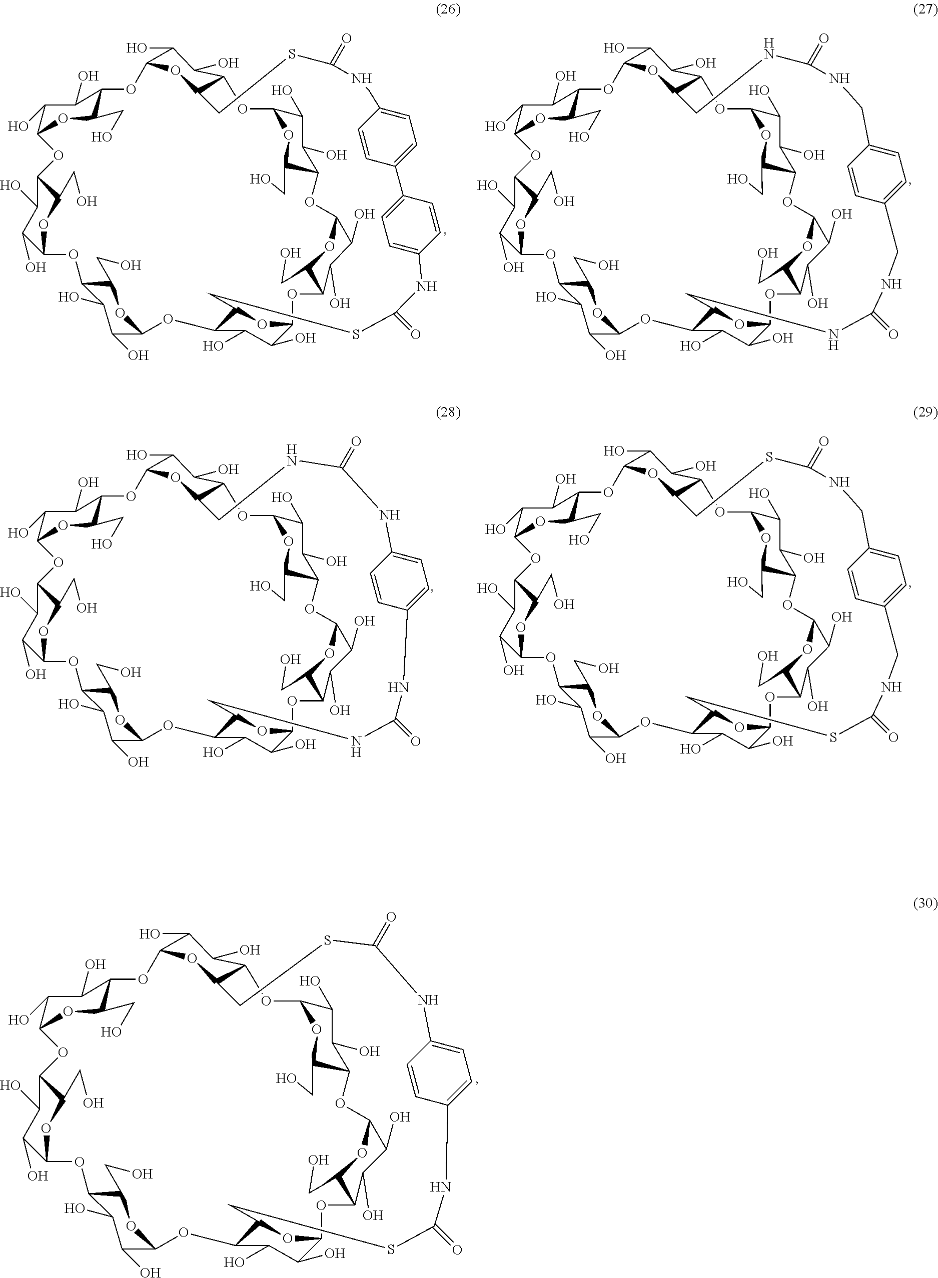

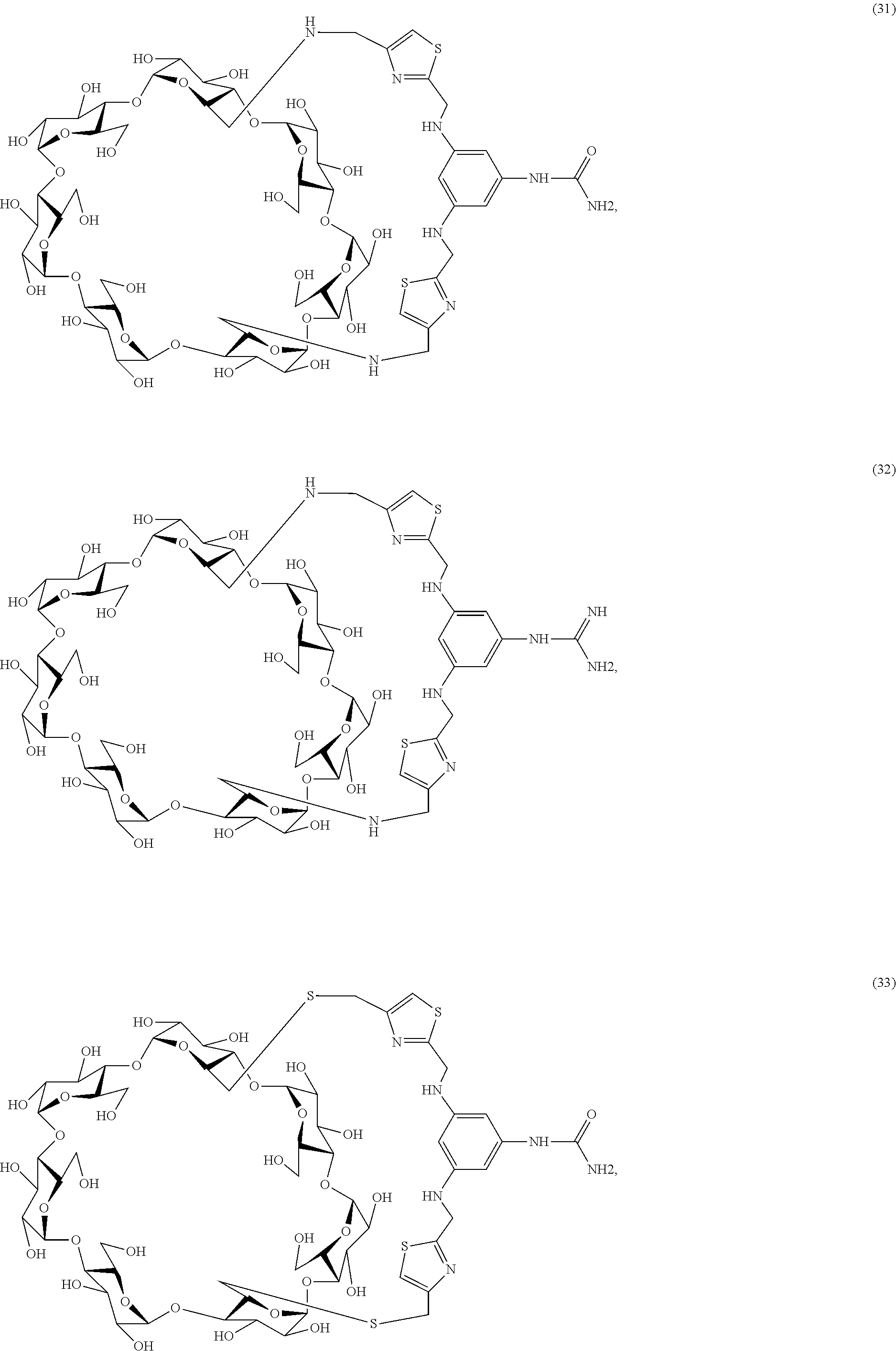

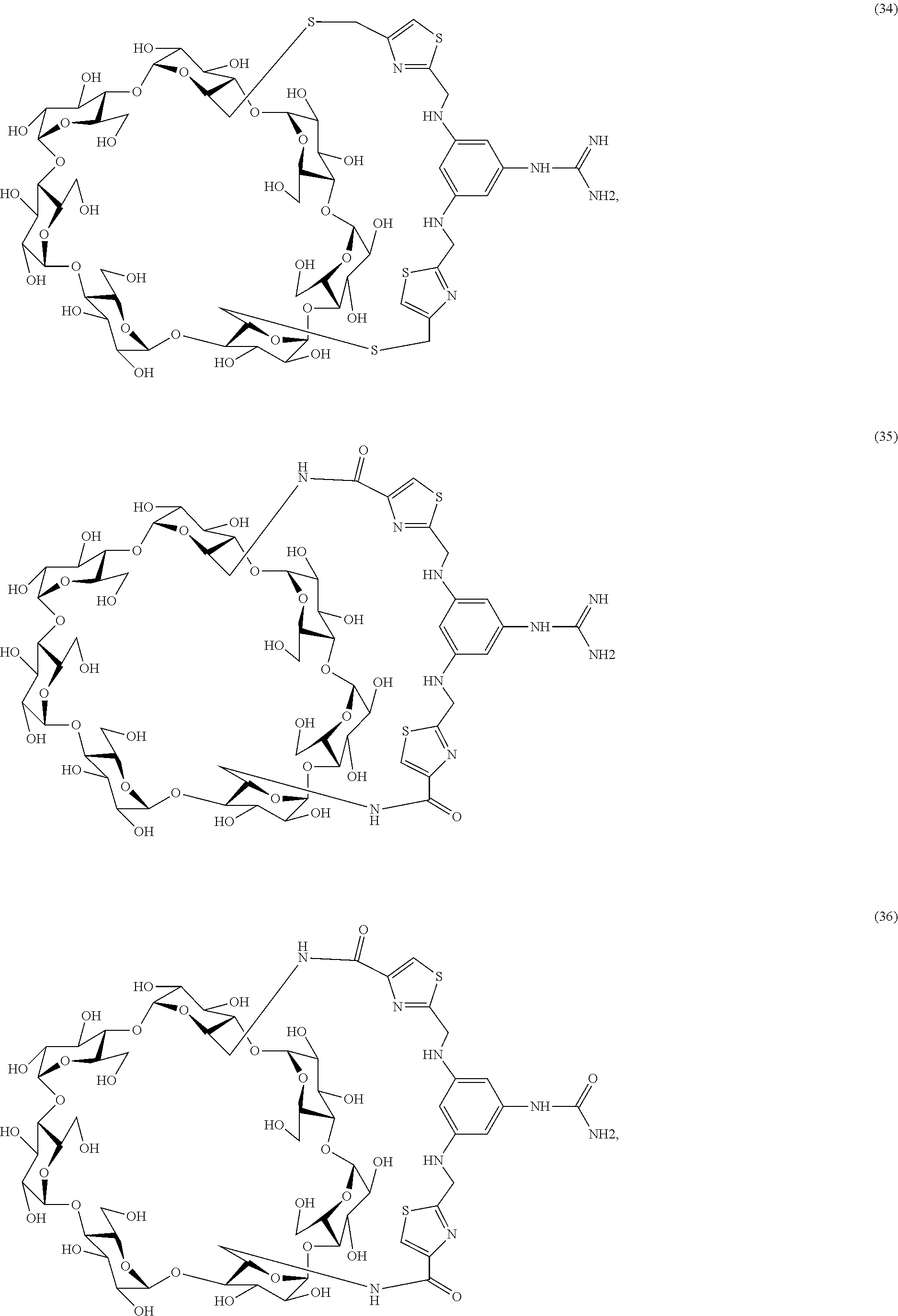

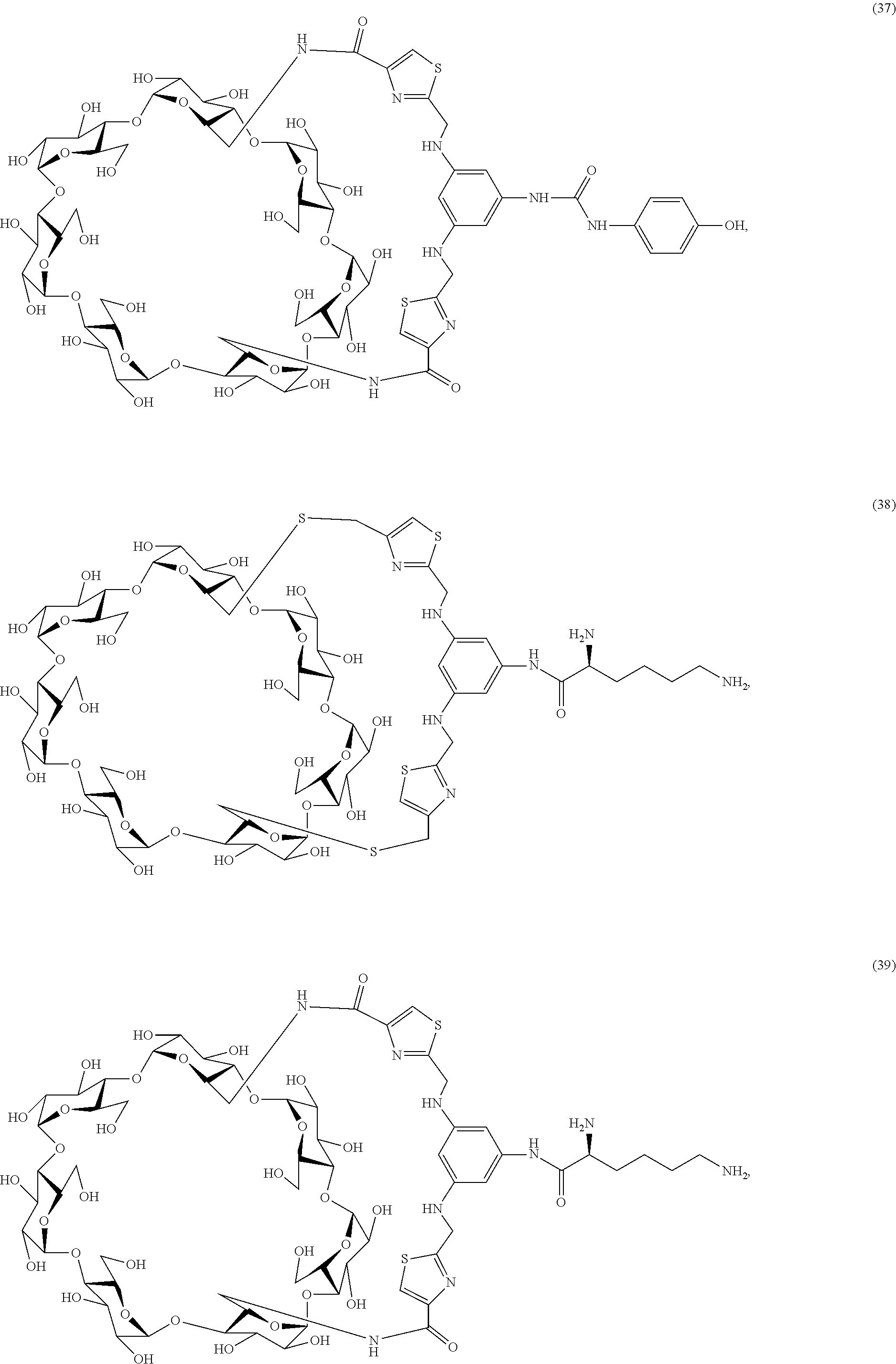

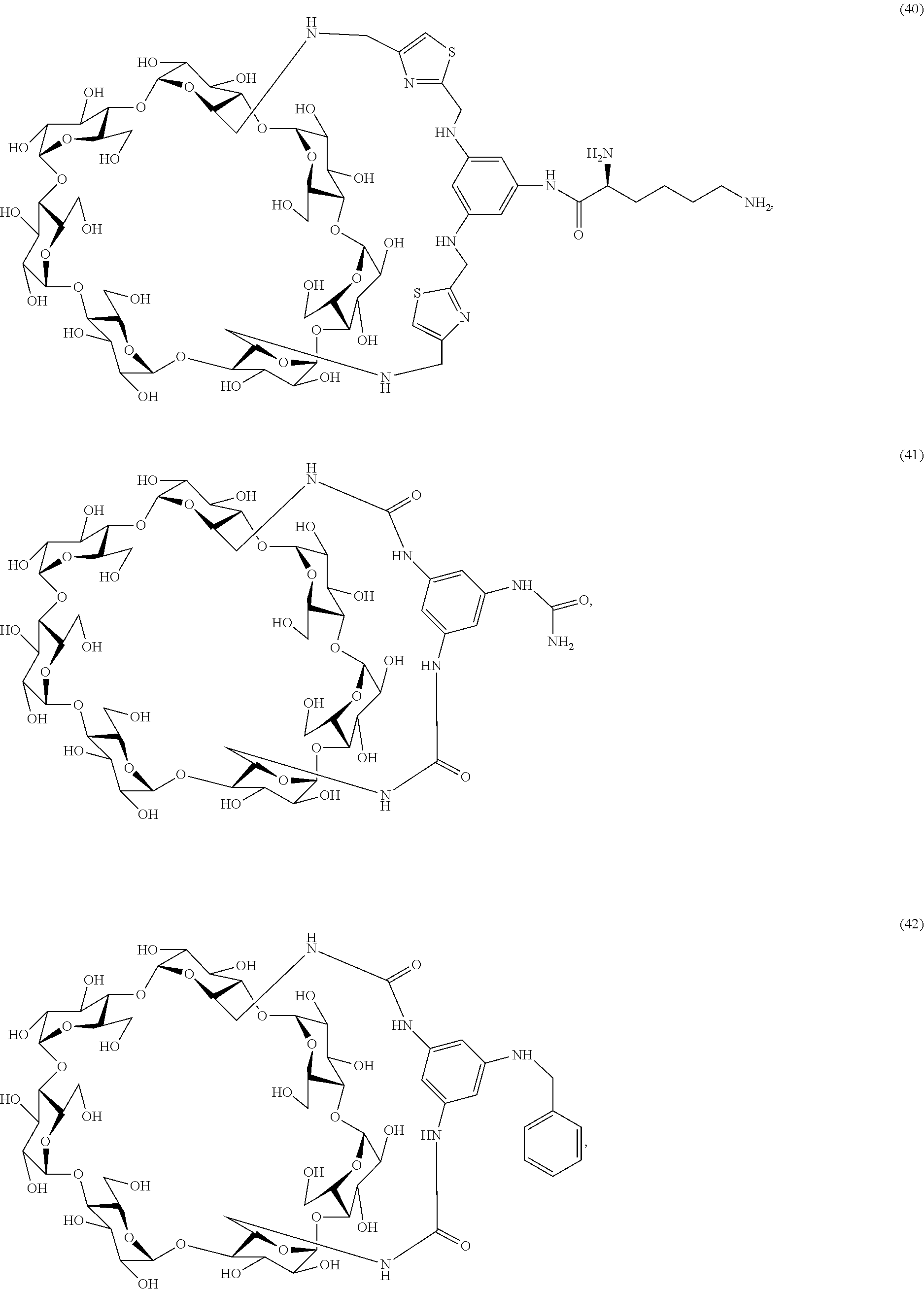

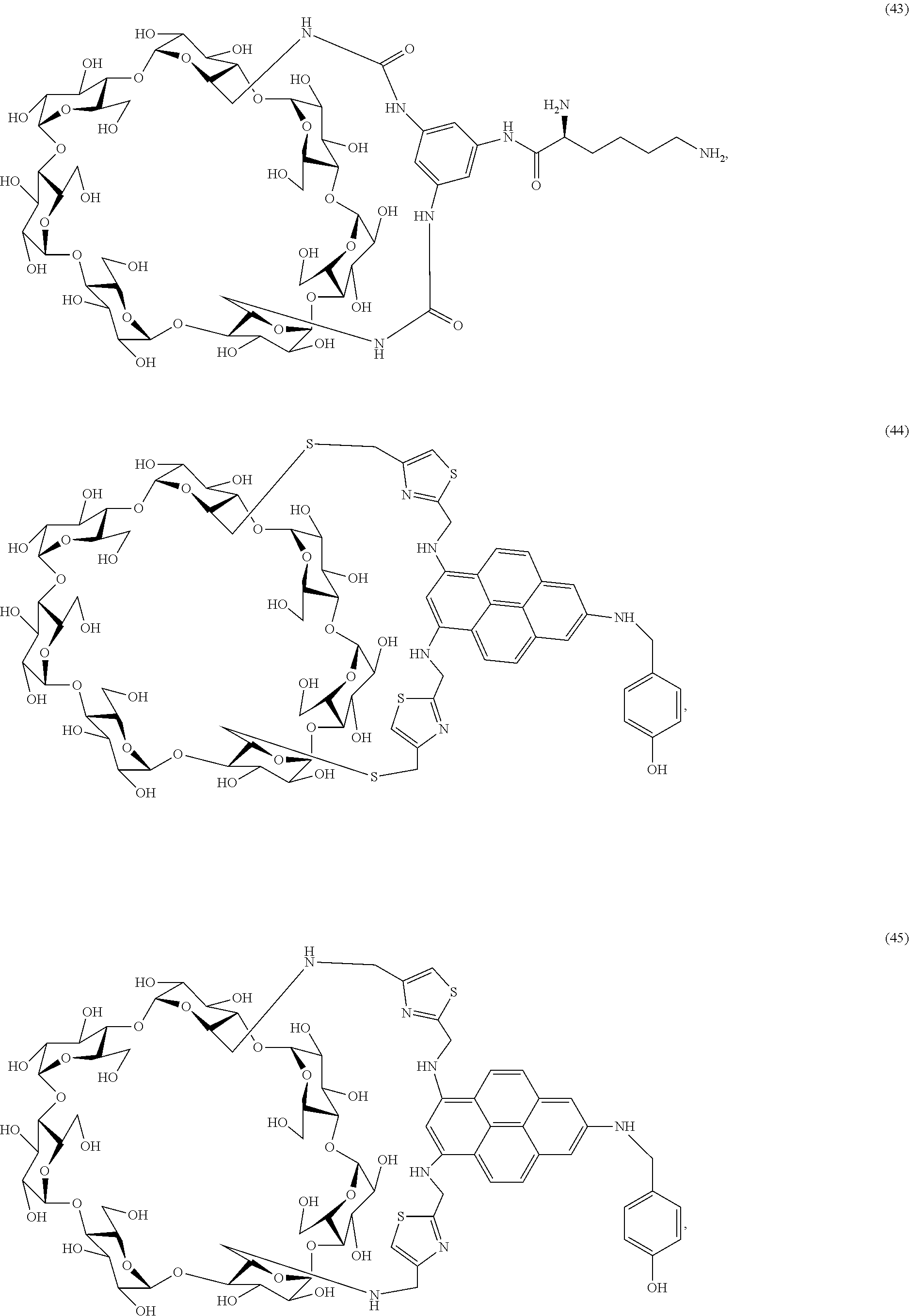

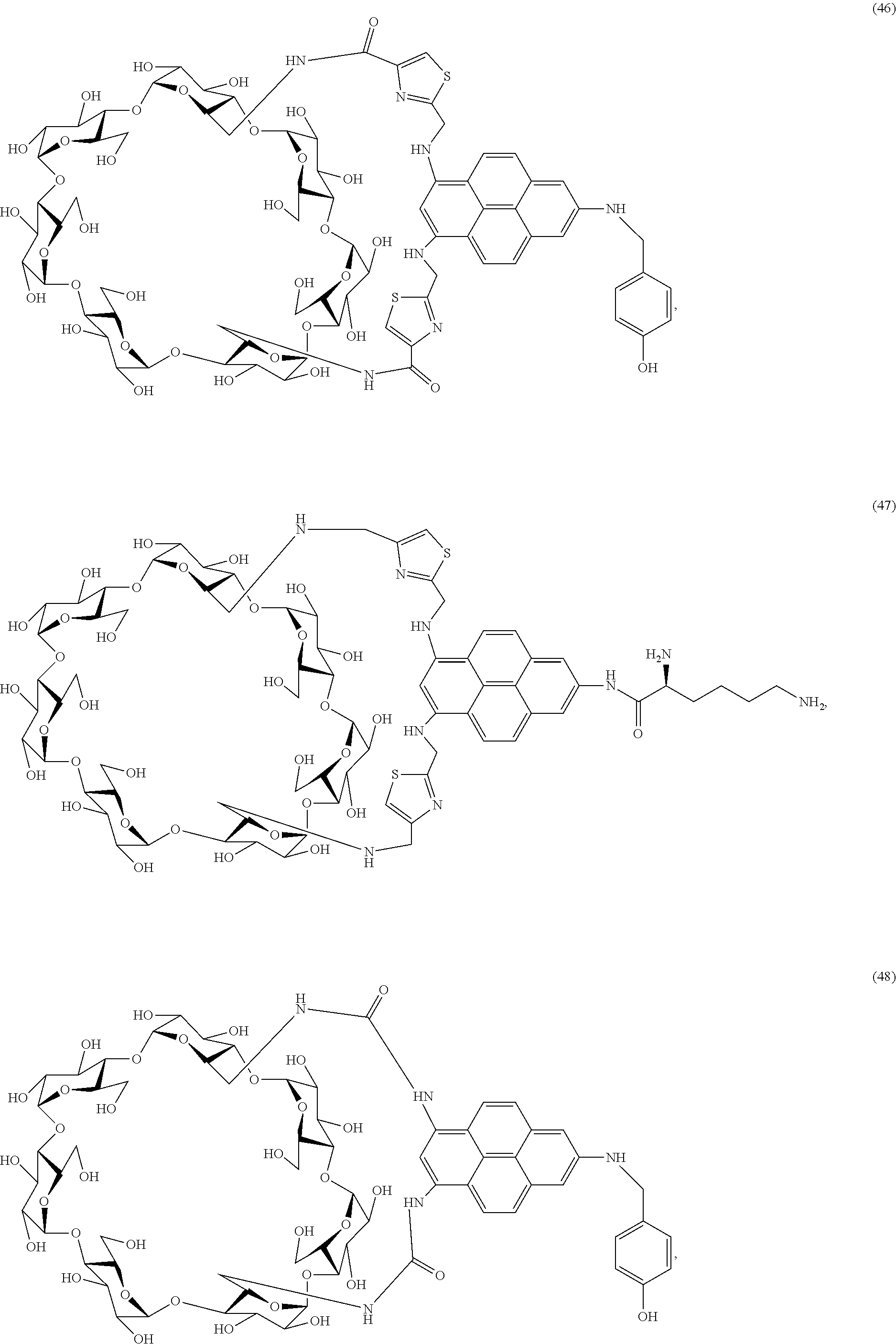

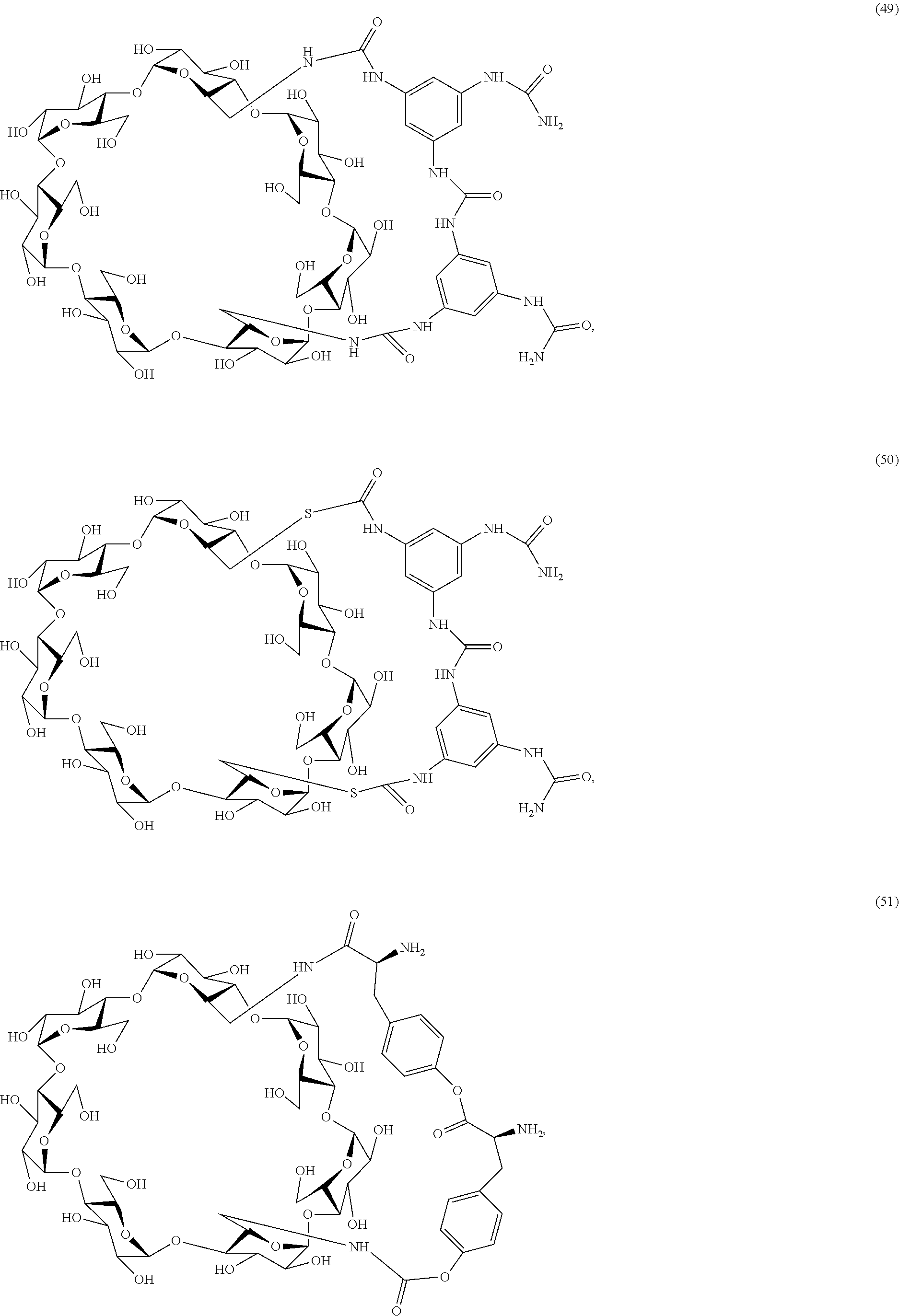

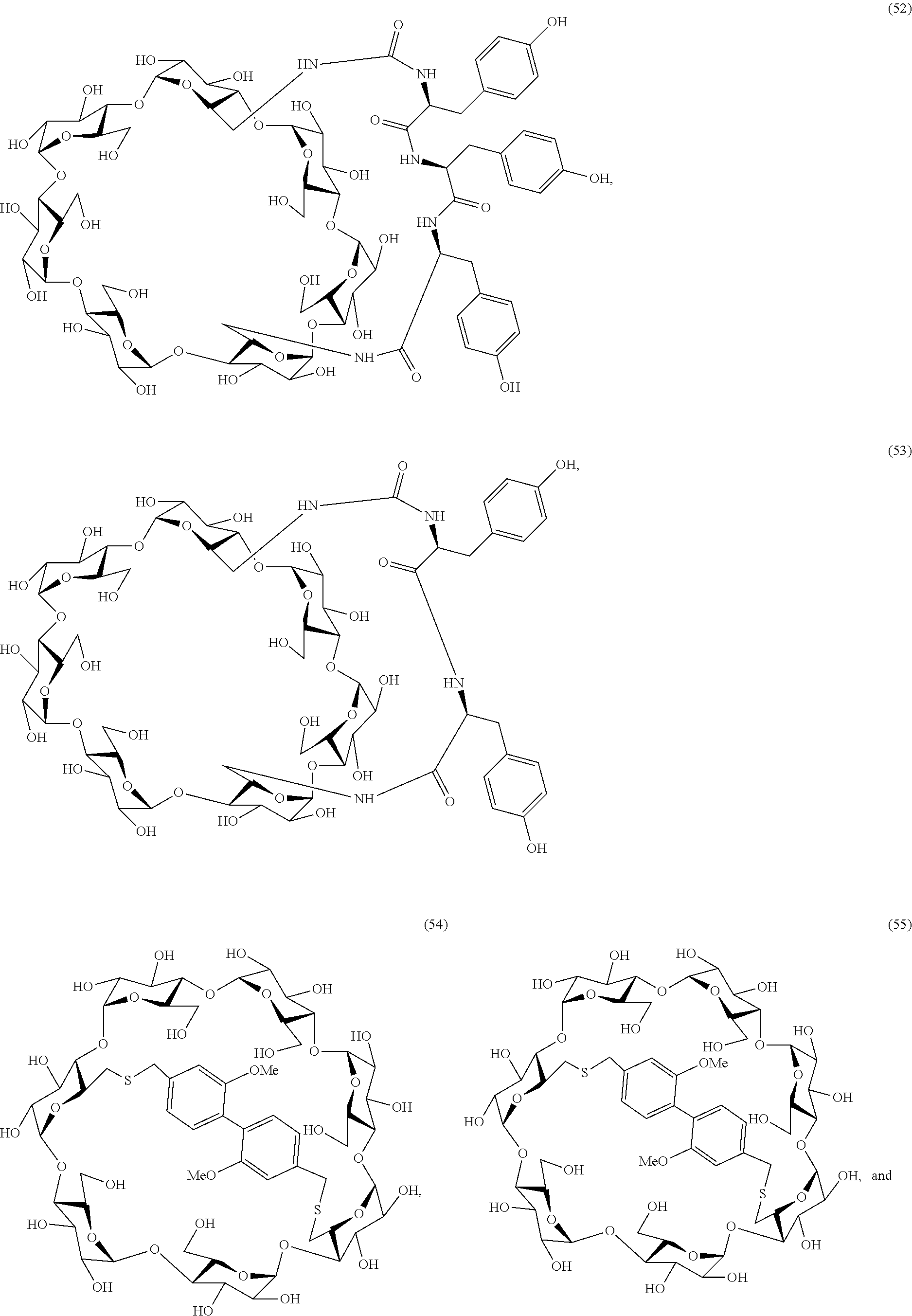

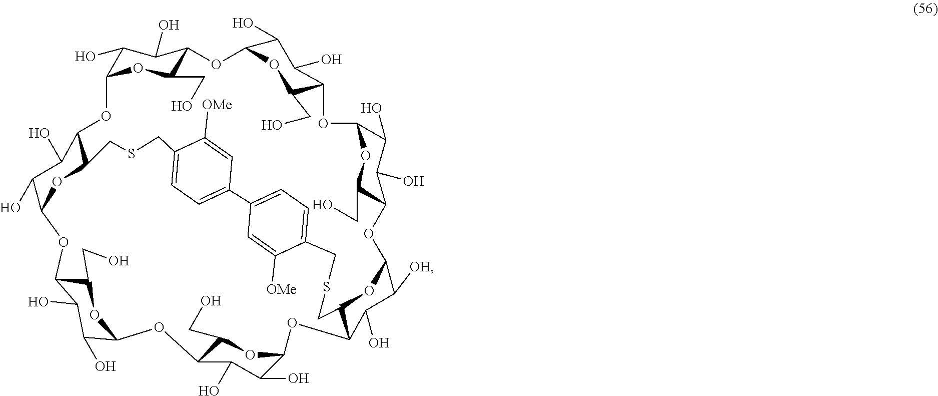

and pharmaceutically acceptable salts, reoisomers, and isotopically labeled derivatives thereof.

In certain embodiments, a compound provided herein is selected from the group consisting of:

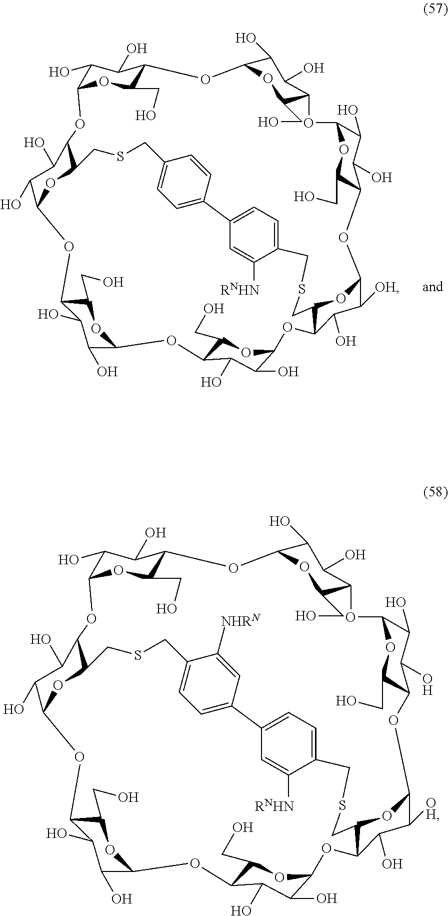

and pharmaceutically acceptable salts, stereoisomers, and isotopically labeled derivatives thereof; wherein each R^{N} is a group of one of the following formulae:

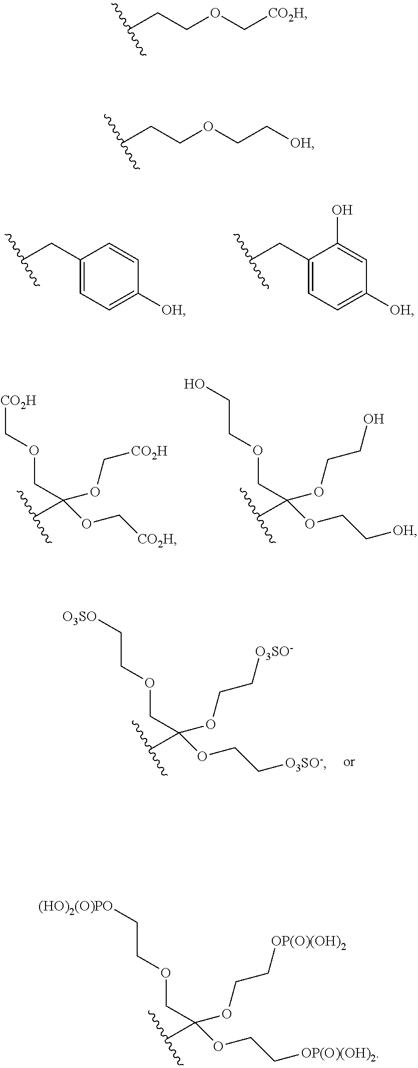

The following definitions apply to all generic and subgeneric formulae described herein (e.g., Formula (I), (II), etc.

m and n

As defined herein, m is an integer between 1 and 10, inclusive. In certain embodiments, m is 1. In certain embodiments, m is 2. In certain embodiments, m is 3, In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, m is an integer from 1 to 5, inclusive. In certain embodiments, m is an integer from 2 to 5, inclusive. In certain embodiments, m is an integer from 3 to 5, inclusive. In certain embodiments, m is an integer from 1 to 4, inclusive. In certain embodiments, m is an integer from 1 to 3, inclusive.

As defined herein, n is an integer between 1 and 10, inclusive. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10. In certain embodiments, n is an integer from 1 to 5, inclusive. In certain embodiments, n is an integer from 2 to 5, inclusive. In certain embodiments, n is an integer from 3 to 5, inclusive. In certain embodiments, n is an integer from 1 to 4, inclusive. In certain embodiments, n is an integer from 1 to 3, inclusive.

In certain embodiments, the sum of m and n is 3. In certain embodiments, the sum of in and n is 4. In certain embodiments, the sum of m and n is 5. In certain embodiments, the sum of m and n is 6. In certain embodiments, the sum of m and n is 7. In certain embodiments, the sum of m and n is 8.

### L and A

As defined herein, L is optionally substituted alkylene, optionally substituted heteroalkylene, optionally substituted alkenylene, optionally substituted alkynylene, optionally substituted carbocyclylene, optionally substituted heterocyclylene, optionally substituted arylene, optionally substituted heteroarylene, optionally substituted acylene, —N(R^{N})—, —O—, —S—, —C(R^{C})₂—, —C(═O)—, —C(═O)N(R^{N})—, —N(R^{N})C(═O)—, —N(R^{N})C(═O)N(R^{N})—, —C(═O)—, —OC(═O)—, —OC(═O)O—, —OC(═O)N(R^{N})—, —N(R^{N})C(═O)O—, or any combination thereof; provided that L comprises one or more moieties selected from the group consisting of optionally substituted aryl, optionally substituted arylene, optionally substituted heteroaryl, optionally substituted heteroarylene, optionally substituted arylalkyl, and optionally substituted heteroarylalkyl.

In certain embodiments, L comprises optionally substituted alkylene. In certain embodiments, L comprises optionally substituted heteroalkylene. In certain embodiments, L comprises optionally substituted alkenylene. In certain embodiments, L comprises optionally substituted alkynylene. In certain embodiments, L comprises optionally substituted carbocyclylene. In certain embodiments, L comprises optionally substituted heterocyclylene. In certain embodiments, L comprises optionally substituted arylene. In certain embodiments, L comprises optionally substituted heteroarylene. In certain embodiments, L comprises optionally substituted acylene, In certain embodiments, L comprises —N(R^{N})—. In certain embodiments, L comprises —O—. In certain embodiments, L comprises —S—. In certain embodiments, L comprises —C(R^{C})₂. In certain embodiments, L comprises —C(═O)—. In certain embodiments, L comprises —C(═O)N(R^{N})—. In certain embodiments, L comprises —N(R^{N})C(═O)—. In certain embodiments, L comprises —N(R^{N})C(═O)N(R^{N})—. In certain embodiments, L comprises —C(═O)O—. In certain embodiments, L comprises —OC(═O)—. In certain embodiments, L comprises —OC(═O)O—. In certain embodiments, L comprises —OC(═O)N(R^{N})—. In certain embodiments, L comprises —N(R^{N})C(═O)O—. In certain embodiments, L comprises —C(═O)S—. In certain embodiments, L comprises —SC(═O)—. In certain embodiments, L comprises —SC(═O)N(R^{N})—. In certain embodiments, L comprises —N(R^{N})C(═O)S—. In certain embodiments, L comprises —SC(═O)O—. In certain embodiments, L comprises —OC(═O)S—.

In certain embodiments, L comprises 1-200 non-hydrogen atoms total. In certain embodiments, L comprises 1-100 non-hydrogen atoms total. In certain embodiments, L comprises 1-50 non-hydrogen atoms total. 111 certain embodiments, L comprises 1-20 non-hydrogen atoms total.

In certain embodiments, L comprises one or more, optionally substituted aryl or optionally substituted arylene moieties. In certain embodiments, L comprises one or more, unsubstituted aryl or unsubstituted arylene moieties. In certain embodiments, L comprises one or more, optionally substituted C₆-C₂₀ aryl or optionally substituted C₆-C₂₀ arylene moieties. In certain embodiments, L comprises one or more, unsubstituted C₆-C₂₀ aryl or unsubstituted C₆-C₂₀ arylene moieties. The optionally substituted aryl or arylene moieties may be tnonocyclic, bicyclic, tricyclic, or polycyclic.

In certain embodiments, L comprises one or more moieties selected from the group consisting of optionally substituted phenyl, optionally substituted phenylene, optionally substituted naphthalenyl, optionally substituted naphthalenylene, optionally substituted anthracenyl, optionally substituted anthracenylene, optionally substituted phenanthrenyl, optionally substituted phenanthrenylene, optionally substituted tetracenyl, optionally substituted tetracenylene, optionally substituted chrysenyl, optionally substituted chrysenyiene, optionally substituted pyrenyl, optionally substituted pyrenyiene, optionally substituted triphenylenyl, optionally substituted triphenylenylene, optionally substituted pentacenyl, optionally substituted pentacenylene, optionally substituted benzo[a]pyrenyl, and optionally substituted benzo[a]pyrenylene.

In certain embodiments, L comprises optionally substituted phenyl. In certain embodiments, L comprises optionally substituted phenylene. In certain embodiments, L comprises unsubstituted phenyl. In certain embodiments, L comprises unsubstituted phenylene.

In certain embodiments, L comprises optionally substituted naphthalenyl. In certain embodiments, L comprises optionally substituted naphthalenylene. In certain embodiments, L comprises unsubstituted naphthalenyl. In certain embodiments, L comprises unsubstituted naphthalenylene.

In certain embodiments, L comprises optionally substituted anthracenyl. In certain embodiments, L comprises optionally substituted anthracenylene. In certain embodiments, L comprises unsubstituted anthracenyl. In certain embodiments, L comprises unsubstituted anthracenylene.

In certain embodiments, L comprises optionally substituted phenanthrenyl. In certain embodiments, L comprises optionally substituted phenanthrenylene. In certain embodiments, L comprises unsubstituted phenanthrenyl. In certain embodiments, L comprises unsubstituted phenanthrenylene.

In certain embodiments, L comprises optionally substituted tetracenyl. In certain embodiments, L comprises optionally substituted tetracenylene. In certain embodiments, L comprises unsubstituted tetracenyl. In certain embodiments, L comprises unsubstituted tetracenylene.

In certain embodiments, L comprises optionally substituted chrysenyl. In certain embodiments, L comprises optionally substituted chrysenylene. In certain embodiments, L comprises unsubstituted chrysenyl. In certain embodiments, L comprises unsubstituted chrysenylene.

In certain embodiments, L comprises optionally substituted pyrenyl. In certain embodiments, L comprises optionally substituted pyrenylene. In certain embodiments, L comprises unsubstituted pyrenyl. In certain embodiments, L comprises unsubstituted pyrenylene.

In certain embodiments, L comprises optionally substituted triphenylenyl. In certain embodiments, L comprises optionally substituted triphenylenylene. In certain embodiments, L comprises unsubstituted triphenylenyl. In certain embodiments, L comprises unsubstituted triphenylenylene.

In certain embodiments, L comprises optionally substituted pentacenyl. In certain embodiments, L comprises optionally substituted pentacenylene. In certain embodiments, L comprises unsubstituted pentacenyl. In certain embodiments, L comprises unsubstituted pentacenylene.

In certain embodiments, L comprises optionally substituted benzo[a]pyrenyl. In certain embodiments, L comprises and optionally substituted benzo[a]pyrenylene. In certain embodiments, L comprises unsubstituted benzo[a]pyrenyl. In certain embodiments, L comprises and unsubstituted benzo[a]pyrenylene.

In certain embodiments, comprises one or more moieties selected from the group consisting of:

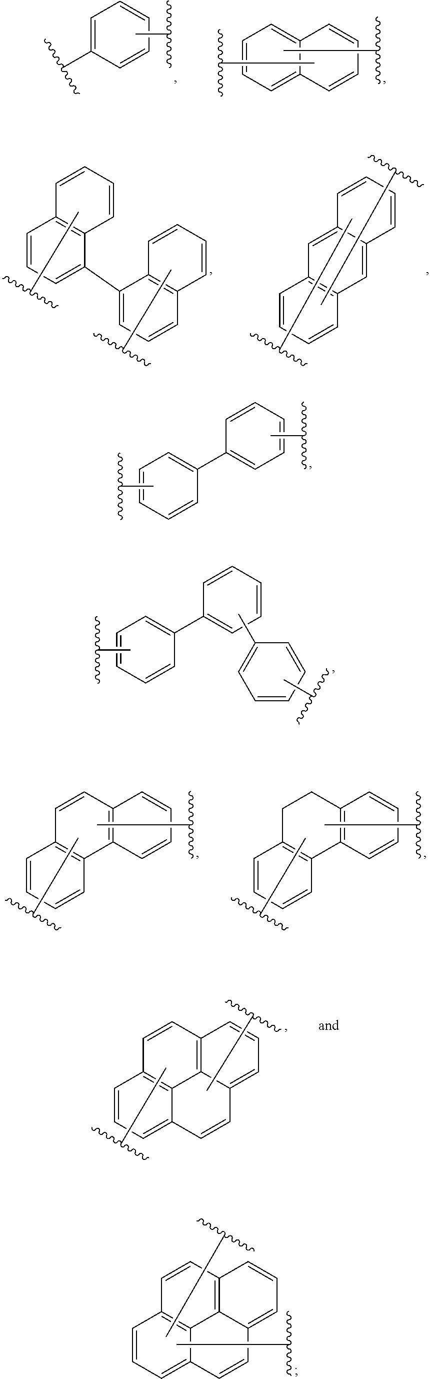

wherein the one or more moieties are optionally substituted. In certain embodiments, the one or more moieties are substituted. In certain embodiments, the one or more moieties are unsubstituted.

In certain embodiments, L comprises one or more moieties selected from the group consisting of:

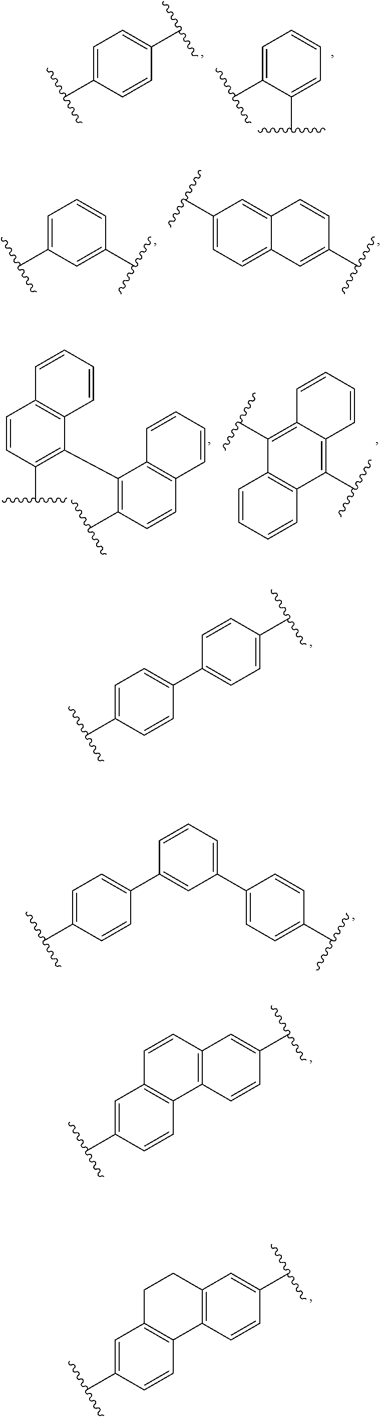

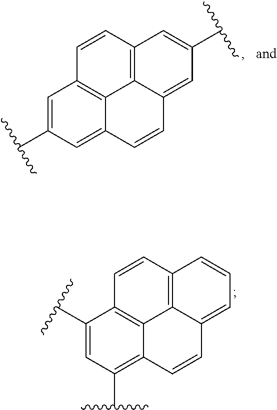 [SMILES: *c1cc2ccc3cc(*)cc4ccc(c1)c2c34 ; *c1cc(*)c2ccc3cccc4ccc1c2c43]

wherein the one or more moieties are optionally substituted. In certain embodiments, the one or more moieties are substituted. In certain embodiments, the one or more moieties are unsubstituted.

In certain embodiments, L comprises one or more moieties selected from the group consisting of:

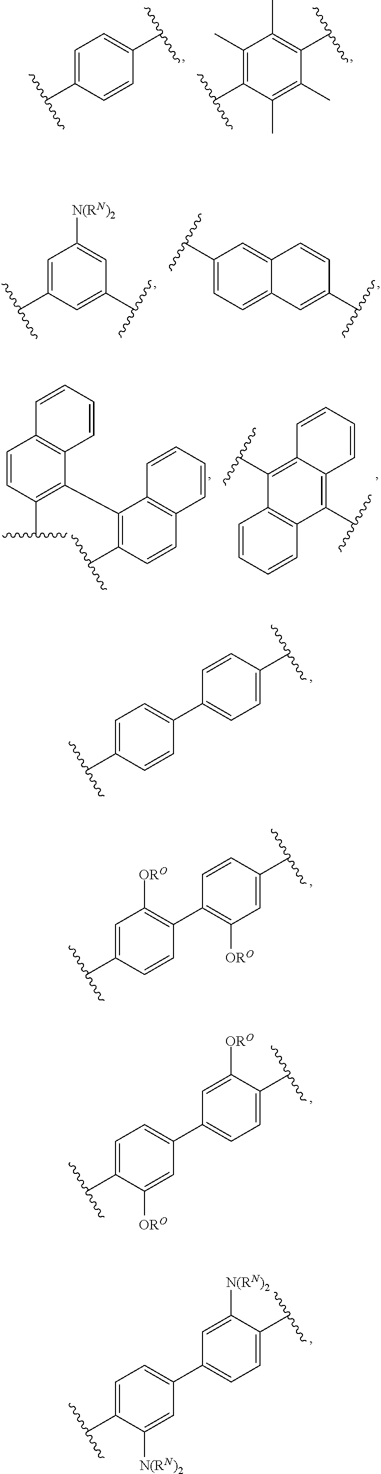

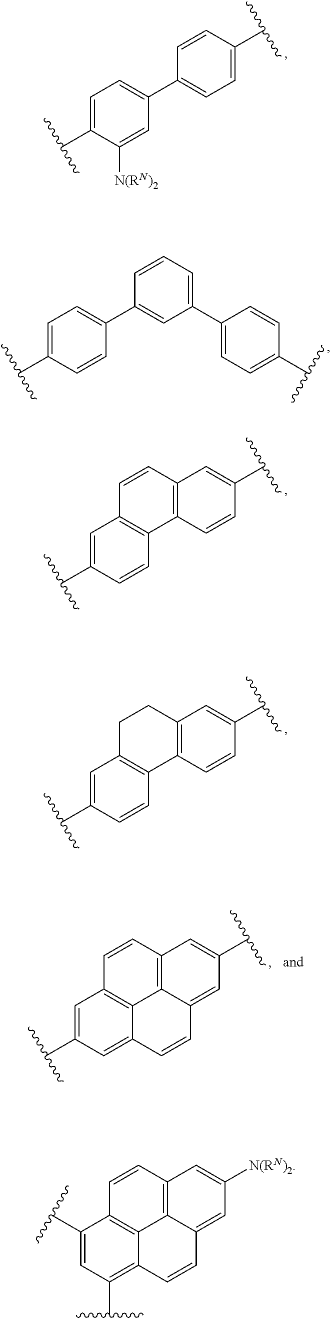

In certain embodiments, L comprises one or more optionally substituted heteroaryl or optionally substituted heteroarylene moieties. In certain embodiments, L comprises one or more unsubstituted heteroaryl or unsubstituted heteroarylene moieties. In certain embodiments, L comprises one or more optionally substituted 5-6 membered heteroaryl or optionally substituted 5-6 membered heteroarylene moieties. In certain embodiments, L comprises one or more unsubstituted 5-6 membered heteroaryl or unsubstituted 5-6 membered heteroarylene moieties. In certain embodiments, L comprises one or more moieties selected from the group consisting of optionally substituted oxazolyl, optionally substituted oxazolylene, optionally substituted thiazolyl, optionally substituted thiazolylene, optionally substituted imidazolyl, and optionally substituted imidazolylene. In certain embodiments, L comprises one or more moieties selected from the group consisting of unsubstituted oxazolyl, unsubstituted oxazolylene, unsubstituted thiazolyl, unsubstituted thiazolylene, unsubstituted imidazolyl, and unsubstituted imidazolylene. In certain embodiments, L comprises optionally substituted thiazolylene. In certain embodiments, L comprises unsubstituted thiazolylene. In certain embodiments, L comprises one or more of the following moieties:

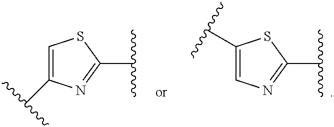 [SMILES: *c1csc(*)n1 ; *c1cnc(*)s1]

In certain embodiments, L comprises one or more optionally substituted arylalkyl or optionally substituted heteroarylalkyl moieties. In certain embodiments, L comprises one or more optionally substituted C₃₋₂₀ arylalkyl or optionally substituted C₃₋₂₀ heteroarylalkyl moieties. In certain embodiments, L comprises one or more unsubstituted C₃₋₂₀ arylalkyl or unsubstituted C₃₋₂₀ heteroarylalkyl moieties. In certain embodiments, L comprises optionally substituted alkyl —C₁₋₆ aryl. In certain embodiments, L comprises unsubstituted alkyl-C₆₋₁₄ aryl. In certain embodiments, L comprises optionally substituted —C₁₋₆ alkyl-phenyl. In certain embodiments, L comprises unsubstituted C₁₋₆ alkyl-phenyl. In certain embodiments, L comprises optionally substituted —C₁₋₃ alkyl-phenyl. In certain embodiments, L comprises unsubstituted —C₁₋₃ alkyl-phenyl. In certain embodiments, L comprises optionally substituted benzyl. In certain embodiments, L comprises unsubstituted benzyl.

In certain embodiments, L comprises one or more amino acids e.g., in a peptide chain). In certain embodiments, L comprises one or more amino acids selected from the group consisting of arginine, histidine, tryptophan, aspartic acid, glutamine, asparagine, glutamine, phenylalanine, and tryptophan. In certain embodiments, L comprises one or more amino acids selected from the group consisting of histidine, tryptophan, and phenylalanine. In certain embodiments, L is a peptidic linker (i.e., comprises one or more peptide bonds in the linker). In certain embodiments, L is a peptidic linker comprising one or more amino acids.

In certain embodiments, L comprises one of the following formulae:

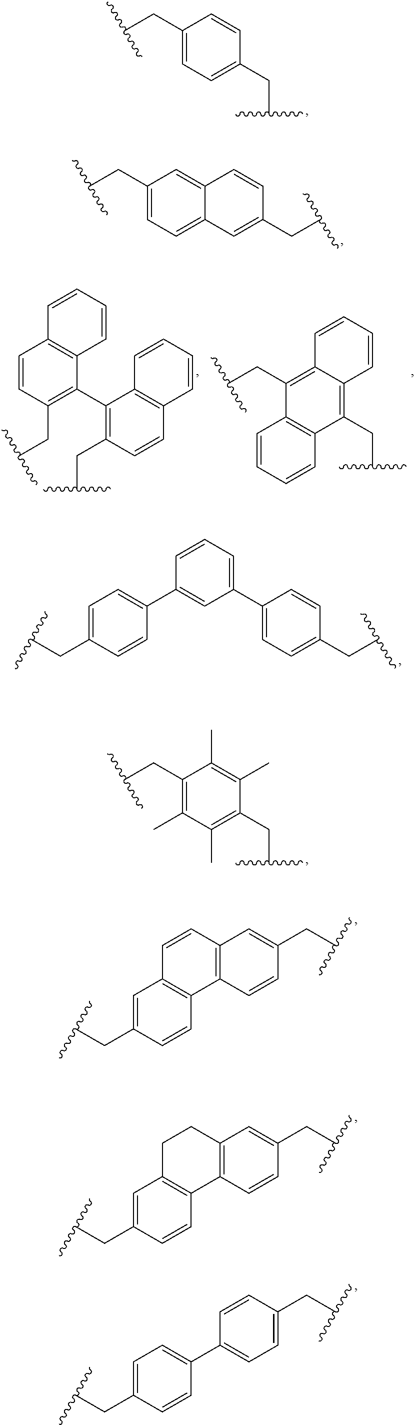

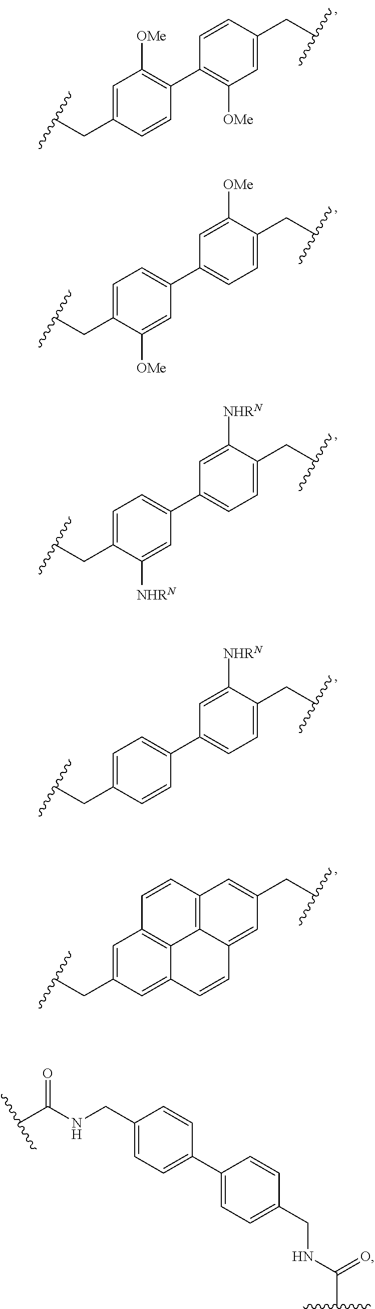

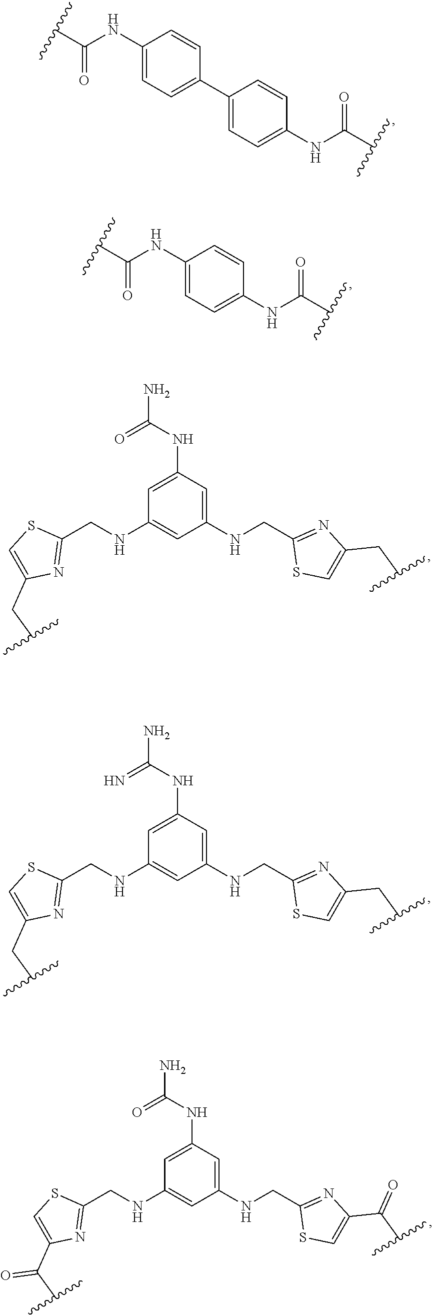 [SMILES: *C(=O)Nc1ccc(-c2ccc(NC(*)=O)cc2)cc1 ; *C(=O)Nc1ccc(NC(*)=O)cc1 ; *Cc1csc(CNc2cc(NCc3nc(C*)cs3)cc(NC(N)=O)c2)n1 ; *Cc1csc(CNc2cc(NCc3nc(C*)cs3)cc(NC(=N)N)c2)n1 ; *C(=O)c1csc(CNc2cc(NCc3nc(C(*)=O)cs3)cc(NC(N)=O)c2)n1]

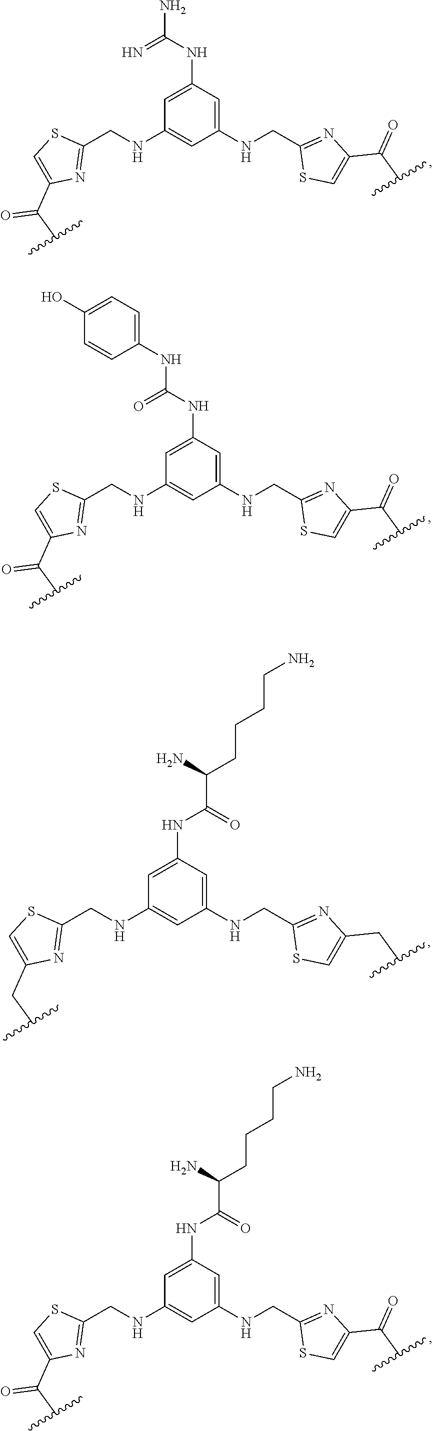 [SMILES: *C(=O)c1csc(CNc2cc(NCc3nc(C(*)=O)cs3)cc(NC(=N)N)c2)n1 ; *C(=O)c1csc(CNc2cc(NCc3nc(C(*)=O)cs3)cc(NC(=O)Nc3ccc(O)cc3)c2)n1 ; *Cc1csc(CNc2cc(NCc3nc(C*)cs3)cc(NC(=O)[C@@H](N)CCCCN)c2)n1 ; *C(=O)c1csc(CNc2cc(NCc3nc(C(*)=O)cs3)cc(NC(=O)[C@@H](N)CCCCN)c2)n1]

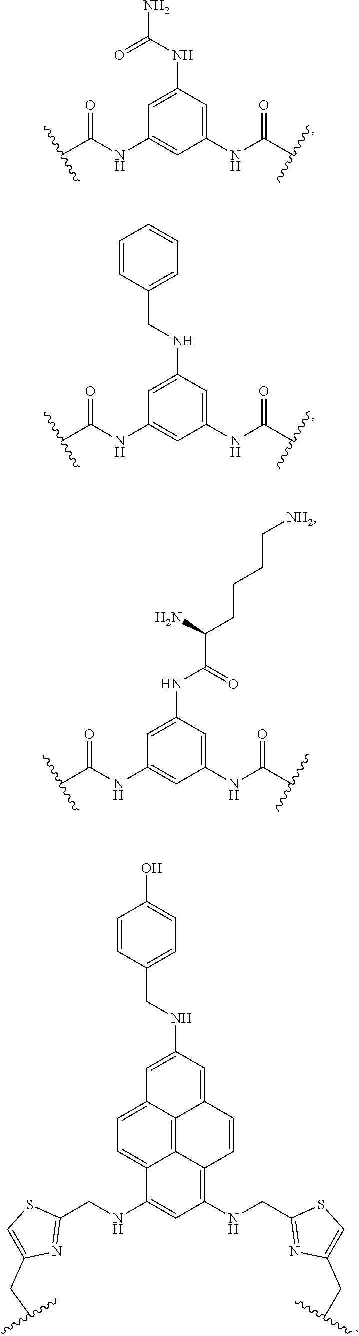 [SMILES: *C(=O)Nc1cc(NC(*)=O)cc(NC(N)=O)c1 ; *C(=O)Nc1cc(NCc2ccccc2)cc(NC(*)=O)c1 ; *C(=O)Nc1cc(NC(*)=O)cc(NC(=O)[C@@H](N)CCCCN)c1 ; *Cc1csc(CNc2cc(NCc3nc(C*)cs3)c3ccc4cc(NCc5ccc(O)cc5)cc5ccc2c3c54)n1]

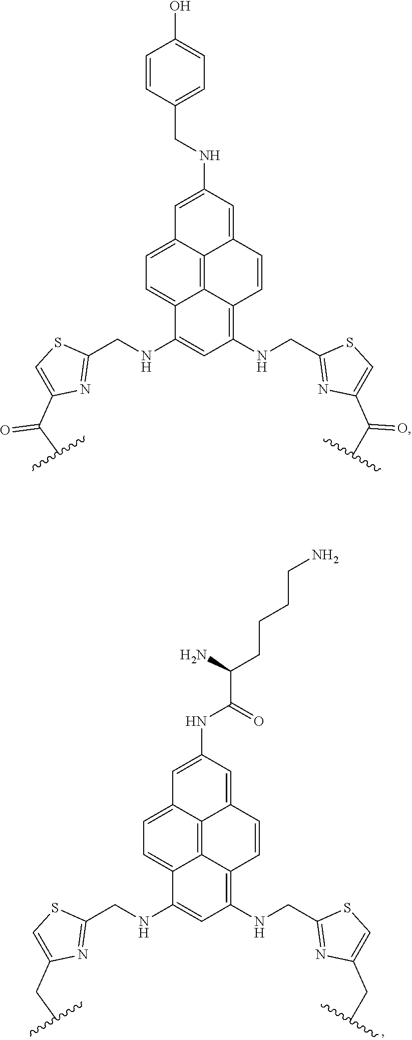 [SMILES: *C(=O)c1csc(CNc2cc(NCc3nc(C(*)=O)cs3)c3ccc4cc(NCc5ccc(O)cc5)cc5ccc2c3c54)n1 ; *Cc1csc(CNc2cc(NCc3nc(C*)cs3)c3ccc4cc(NC(=O)[C@@H](N)CCCCN)cc5ccc2c3c54)n1]

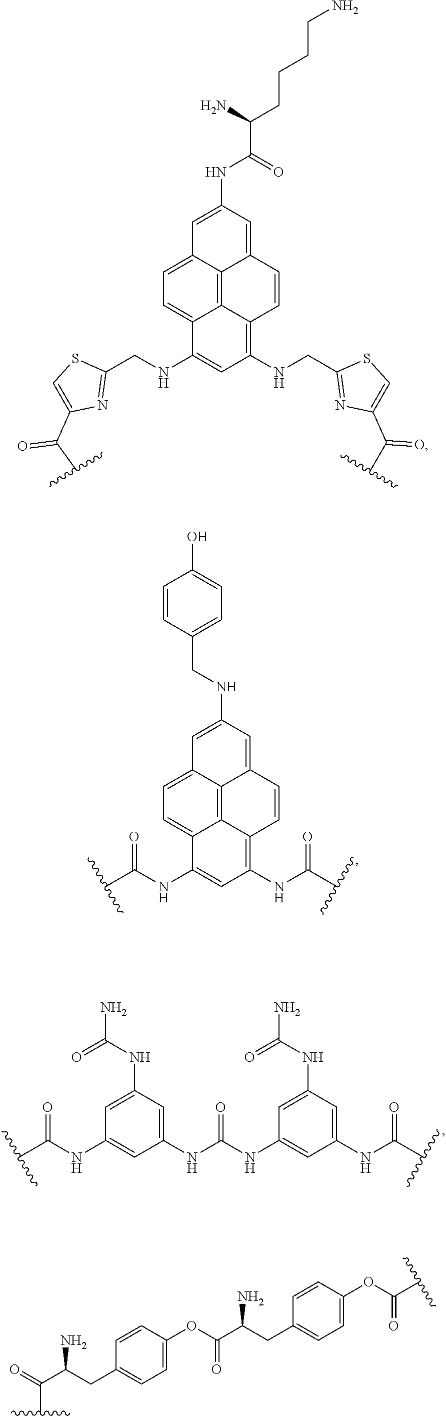

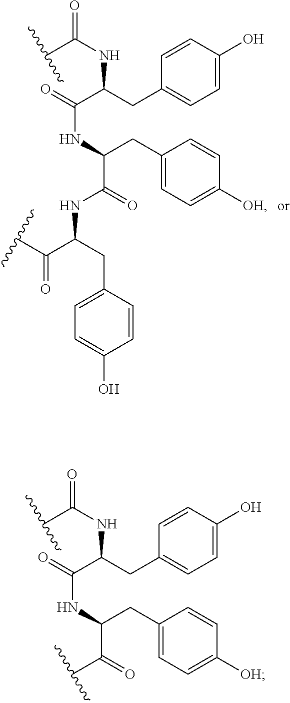 [SMILES: *C(=O)N[C@@H](Cc1ccc(O)cc1)C(=O)N[C@@H](Cc1ccc(O)cc1)C(=O)N[C@@H](Cc1ccc(O)cc1)C(*)=O ; "or" ; *C(=O)N[C@@H](Cc1ccc(O)cc1)C(=O)N[C@@H](Cc1ccc(O)cc1)C(*)=O]

wherein each instance of R^{N} is independently hydrogen or a group of one of the following formulae:

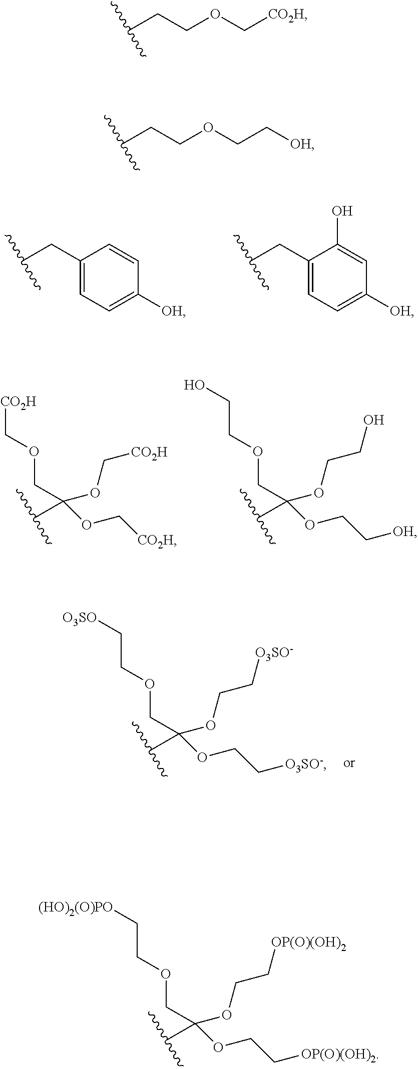

In certain embodiments, L is one of the preceding formulae.

As defined herein, each instance of A is independently a bond, optionally substituted alkylene, optionally substituted heteroalkylene, —O—, —N(R^{N})—, —C(R^{C})₂—, —C(═O)—, —C(R^{C})₂O—, —C(R^{C})₂N(R^{N})—, or —C(R^{C})₂S—. In certain embodiments, at least one instance of A is a bond. In certain embodiments, at least one instance of A is optionally substituted alkylene. In certain embodiments, at least one instance of A is optionally substituted heteroalkylene. In certain embodiments, at least one instance of A is —O—. In certain embodiments, at least one instance of A is —N(R^{N})—. In certain embodiments, at least one instance of A is —S—. In certain embodiments, at least one instance of A is —C(R^{C})₂—. In certain embodiments, at least one instance of A is —C(═O)—. In certain embodiments, at least one instance of A is —C(R^{C})₂O—. In certain embodiments, at least one instance of A is —C(R^{C})₂N(R^{N})—. In certain embodiments, at least one instance of A is or —C(R^{C})₂S—.

In certain embodiments, at least one instance of A is optionally substituted C₁₋₃ heteroalkylene. In certain embodiments, at least one instance of A is unsubstituted C₁₋₃ heteroalkylene. In certain embodiments, at least one instance of A is —C(R^{C})₂O—, —C(R^{C})₂N(R^{N})—, or —C(R^{C})₂S—. In certain embodiments, at least one instance of A is —C(R^{C})₂O—. In certain embodiments, at least one instance of A is —C(R^{C})₂N(R^{N})—. In certain embodiments, at least one instance of A is or —C(R^{C})₂S—. In certain embodiments, at least one instance of A is —CH₂S—, or —CH₂N(R^{N})—. In certain embodiments, at least one instance of A is CH₂O. In certain embodiments, at least one instance of A is —CH₂S—. In certain embodiments, at least one instance of A is —CH₂N(R^{N})—. In certain embodiments, at least one instance of A is —CH₂NH—.

In certain embodiments, each instance of A is —CH₂O—. In certain embodiments, each instance of A is —CH₂S—. In certain embodiments, each instance of A is —CH₂N(R^{N})—. In certain embodiments, each instance of A is —CH₂NH—.

In certain embodiments, each instance of A is the same. In certain embodiments, each instance of A is different.

### Y and R³

As defined herein, each instance of Y is independently a bond, —C(R^{C})₂—, —O—, —S—, or —N(R^{N})—. In certain embodiments, Y is independently —O—, —S—, or —N(R^{N})—. In certain embodiments, at least one instance of Y is a bond. In certain embodiments, at least one instance of Y is —C(R^{C})₂—. In certain embodiments, at least one instance of Y is —O—. In certain embodiments, at least one instance of Y is —S—. In certain embodiments, at least one instance of Y is —N(R^{N})—. In certain embodiments, at least one instance of Y is —NH—. In certain embodiments, at least one instance of Y is —CH₂—.

In certain embodiments, each instance of Y is —O—. In certain embodiments, each instance of Y is —S—. In certain embodiments, each instance of Y is —N(R^{N})—. In certain embodiments, each instance of Y is —NH—. In certain embodiments, each instance of Y is —CH₂—.

As defined herein, each instance of R³ is independently —OR^{O}—, —N(R^{N})₂—, or —SR^{S}—. In certain embodiments, at least one instance of R³ is —OR^{O}—. In certain embodiments, at least one instance of R³ is —N(R^{N})₂. In certain embodiments, at least one instance of R³ is —SR^{S}—. In certain embodiments, at least one instance of R³ is —OH. In certain embodiments, at least one instance of R³ is —NH₂. In certain embodiments, at least one instance of R³ is —SH.

In certain embodiments, each instance of R³ is —OR^{O}. In certain embodiments, each instance of R³ is —N(R^{N})₂. In certain embodiments, each instance of R³ is —SR^{S}. In certain embodiments, each instance of R³ is —OH. In certain embodiments, each instance of R³ is —NH₂. In certain embodiments, each instance of R³ is —SH.

R¹ and R²

As defined herein, each instance of is independently hydrogen, optionally substituted alkyl, optionally substituted carbocyclyl, optionally substituted heterocyclyl, optionally substituted aryl, optionally substituted heteroaryl, optionally substituted optionally substituted acyl, an oxygen protecting group, or a sugar. In certain embodiments, at least one instance of R¹ is hydrogen. In certain embodiments, at least one instance of R¹ is optionally substituted alkyl. In certain embodiments, at least one instance of R¹ is optionally substituted carbocyclyl. In certain embodiments, at least one instance of R¹ is optionally substituted heterocyclyl. In certain embodiments, at least one instance of R¹ is optionally substituted aryl. In certain embodiments, at least one instance of R¹ is optionally substituted heteroaryl. In certain embodiments, at least one instance of R¹ is optionally substituted optionally substituted acyl. In certain embodiments, at least one instance of R¹ is an oxygen protecting group. In certain embodiments, at least one instance of is a sugar.

In certain embodiments, at least one instance of R¹ is optionally substituted C₁₋₆ alkyl. In certain embodiments, at least one instance of is unsubstituted C₄₋₆ alkyl. In certain embodiments, at least one instance of R¹ is optionally substituted C₁₋₃ alkyl. In certain embodiments, at least one instance of R¹ is unsubstituted C₁₋₃ alkyl. In certain embodiments, at least one instance of R¹ is methyl, ethyl, n-propyl, iso-propyl, n-butyl, sec-butyl, iso-butyl, or tert-butyl.

In certain embodiments, at least one instance of R¹ is hydrogen. In certain embodiments, each instance of R¹ is hydrogen.

As defined herein, each instance of R² is independently optionally substituted alkyl, optionally substituted heteroalkyl, optionally substituted carbocyclyl, optionally substituted heterocyclyl, optionally substituted aryl, optionally substituted heteroaryl, optionally substituted acyl, —OR^{O}, —N(R^{N})₂, —SR^{S}, —C(R^{C})₂OR^{O}, —C(R^{C})₂N(R^{N})₂, —C(R^{C})₂SR^{S}, or a sugar. In certain embodiments, at least one instance of R² is optionally substituted alkyl. In certain embodiments, at least one instance of R² is optionally substituted heteroalkyl. In certain embodiments, at least one instance of R² is optionally substituted carbocyclyl. In certain embodiments, at least one instance of R² is optionally substituted heterocyclyl. In certain embodiments, at least one instance of R² is optionally substituted aryl. In certain embodiments, at least one instance of R² is optionally substituted heteroaryl, In certain embodiments, at least one instance of R² is optionally substituted acyl. In certain embodiments, at least one instance of R² is —OR^{O}. In certain embodiments, at least one instance of R² is —N(R^{N})₂. In certain embodiments, at least one instance of R² is —SR^{S}. In certain embodiments, at least one instance of R² is —C(R^{C})OR^{O}. In certain embodiments, at least one instance of R² is —C(R^{C})₂N(R^{N})₂. In certain embodiments, at least one instance of R² is —C(R^{C})₂SR^{S}. In certain embodiments, at least one instance of R² is a sugar. In certain embodiments, at least one instance of R² is —CH₂OR^{O}. In certain embodiments, at least one instance of R² is —CH₂OH. In certain embodiments, at least one instance of R² is —CH₂N(R^{N})₂. In certain embodiments, at least one instance of R² is —CH₂NH₂.

In certain embodiments, each instance of R² is —CH₂OR^{O}. In certain embodiments, each instance of R² is —CH₂OH. In certain embodiments, each instance of R² is —CH₂N(R^{N})₂. In certain embodiments, each instance of R² is —CH₂NH₂.

R^{O}, R^{N}, and R^{C}

As defined herein, each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group, In certain embodiments, at least one instance of R^{O} is optionally substituted alkyl. In certain embodiments, at least one instance of R^{O} is optionally substituted alkenyl. In certain embodiments, at least one instance of R^{O} is optionally substituted alkynyl. In certain embodiments, at least one instance of R^{O} is optionally substituted carbocyclyl. In certain embodiments, at least one instance of R^{O} is optionally substituted aryl. In certain embodiments, at least one instance of R^{O} is optionally substituted heterocyclyl. In certain embodiments, at least one instance of R^{O} is optionally substituted heteroaryl. In certain embodiments, at least one instance of R^{O} is optionally substituted acyl. In certain embodiments, at least one instance of R^{O} is an oxygen protecting group.

In certain embodiments, at least one instance of R^{O} is optionally substituted C₁₋₆ alkyl. In certain embodiments, at least one instance of R^{O} is unsubstituted C₁₋₆ alkyl. In certain embodiments, at least one instance of R^{O} is optionally substituted C₁₋₃ alkyl. In certain embodiments, at least one instance of R^{O} is unsubstituted C₁₋₃ alkyl. In certain embodiments, at least one instance of R^{O} is methyl, ethyl, n-propyl, iso-propyl, n-butyl, sec-butyl, iso-butyl, or tert-butyl.

In certain embodiments, at least one instance of R^{O} is hydrogen. In certain embodiments, each instance of R^{O} is hydrogen.

In certain embodiments, at least one instance of R^{O} is of one of the following formulae:

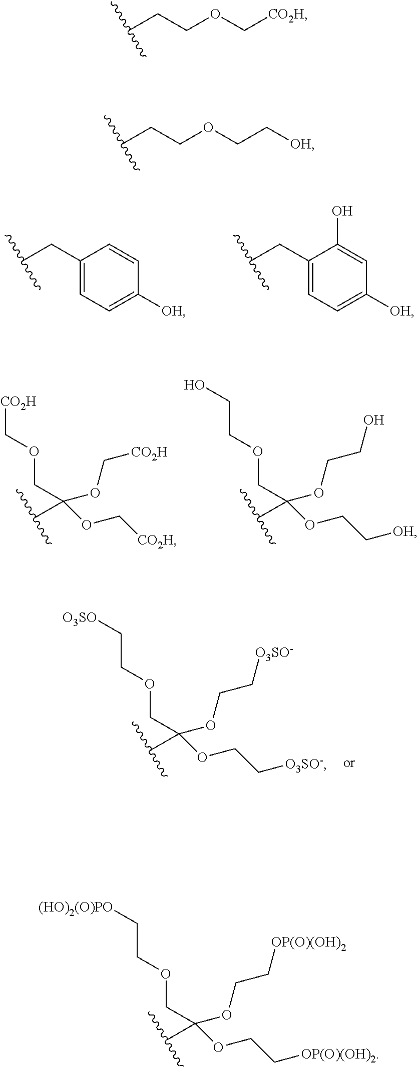

As defined herein, each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or wherein two R^{N} groups attached to the same nitrogen atom are taken together with the intervening atoms to form optionally substituted heterocyclyl or optionally substituted heteroaryl. In certain embodiments, at least one instance of R^{N} is hydrogen. In certain embodiments, at least one instance of R^{N} is optionally substituted alkyl. In certain embodiments, at least one instance of R^{N} is optionally substituted alkenyl. In certain embodiments, at least one instance of R^{N} is optionally substituted alkynyl. In certain embodiments, at least one instance of R^{N} is optionally substituted carbocyclyl. In certain embodiments, at least one instance of R^{N} is optionally substituted aryl. In certain embodiments, at least one instance of R^{N} is optionally substituted heterocyclyl. In certain embodiments, at least one instance of R^{N} is optionally substituted heteroaryl. In certain embodiments, at least one instance of R^{N} is optionally substituted acyl. In certain embodiments, at least one instance of R^{N} is or a nitrogen protecting group. In certain embodiments two R^{N} groups attached to the same nitrogen atom are taken together with the intervening atoms to form optionally substituted heterocyclyl. In certain embodiments two R^{N} groups attached to the same nitrogen atom are taken together with the intervening atoms to form optionally substituted heteroaryl.

In certain embodiments, at least one instance of R^{N} is optionally substituted C₁₋₆ alkyl. In certain embodiments, at least one instance of R^{N} is unsubstituted C₁₋₆ alkyl. In certain embodiments, at least one instance of R^{N} is optionally substituted C₁₋₃ alkyl. In certain embodiments, at least one instance of R^{N} is unsubstituted alkyl. In certain embodiments, at least one instance of R^{N} is methyl, ethyl, n-propyl, iso-propyl, n-butyl, sec-butyl, iso-butyl, or tert-butyl.

In certain embodiments, at least one instance of R^{N} is of one of the following formulae:

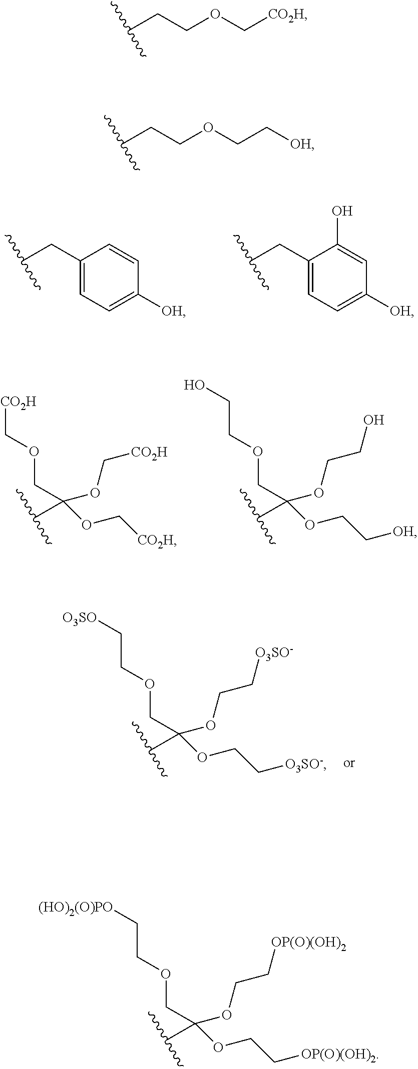

In certain embodiments, both instances of R^{N} on a nitrogen atom is hydrogen. In certain embodiments, one instance of R^{N} is hydrogen, and the other is a non-hydrogen group. In certain embodiments, both instances R^{N} on a nitrogen atom is a non-hydrogen group.

As defined herein, each instance of R^{C} is independently hydrogen, halogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —N(R^{N})₂, or —SR^{S}; or two R^{C} are taken together to form an oxo group. In certain embodiments, at least one instance of R^{C} is halogen. In certain embodiments, at least one instance of R^{C} is optionally substituted alkyl. In certain embodiments, at least one instance of R^{C} is optionally substituted alkenyl. In certain embodiments, at least one instance of R^{C} is optionally substituted alkynyl. In certain embodiments, at least one instance of R^{C} is optionally substituted carbocyclyl. In certain embodiments, at least one instance of R^{C} is optionally substituted aryl. In certain embodiments, at least one instance of R^{C} is optionally substituted heterocyclyl. In certain embodiments, at least one instance of R^{C} is optionally substituted heteroaryl. In certain embodiments, at least one instance of R^{C} is optionally substituted acyl. In certain embodiments, at least one instance of R^{C} is —CN. In certain embodiments, at least one instance of R^{C} is —OR^{O}. In certain embodiments, at least one instance of R^{C} is —N(R^{N})₂. In certain embodiments, at least one instance of R^{C} is —SR^{S}. In certain embodiments, two R^{C} are taken together to form an oxo group.

In certain embodiments, at least one instance of R^{C} is hydrogen. In certain embodiments, each instance of R^{C} is hydrogen.

### Conjugates

A compound provided here e.g., a cyclodextrin-based compound) can be conjugated to an agent or a tag. Therefore, provided herein are conjugates comprising a compound provided herein, or a pharmaceutically acceptable salt, stereoisomer, or isotopically labeled derivative thereof, conjugated to an agent or a tag.

In certain embodiments, the compound is conjugated to an agent. In certain embodiments, the agent is a protein, peptide, nucleic acid, polysaccharide, lipid, or small molecule. In certain embodiments, the agent is a diagnostic agent, imaging agent, therapeutic agent, or prophylactic agent. In certain embodiments, the agent is a protein. In certain embodiments, the agent is a therapeutic protein. In certain embodiments, the protein is insulin (e.g., wild-type or modified insulin), In certain embodiments, the protein is wild-type insulin (i.e., native insulin). In certain embodiments, the protein is modified insulin (i.e., an insulin analog). In certain embodiments, a compound provided herein is conjugated to insulin to form a glucose-responsive form of insulin. In general, “glucose-responsive insulin” is a modified insulin wherein the activity of the insulin is proportional to the glycemic level (i.e., the concentration of glucose in the blood) of a subject. See the Examples for certain embodiments of glucose responsive insulin.

In certain embodiments, the compound is conjugated to a tag. In certain embodiments, the tag is selected from radionuclides, fluorophores, chromophores, phosphorescent agents, dyes, chemiluminescent agents, colorimetric labels, magnetic labels, haptens, and excipients. In certain embodiments, the compound is conjugated to form a glucose-sensing material. In certain embodiments, the conjugate can be used in glucose detection and/or monitoring.

As described herein, in certain embodiments, the compounds described herein can be used to construct glucose sensing devices. For example, in some embodiments, there are three components of the assembly: a detector, a transducer, and a reporter. For example, the compounds can be attached to fluorophores or carbon nanotubes and the binding events (e.g., binding to glucose) can be utilized to provide fluorescent or voltammetric readout.

The agent or tag may be conjugated to the compound via any positon on the compound. For example, the agent or tag may be conjugated from a sugar or from the crosslink of the macrocycle. The compound may be conjugated to one agent or tag, or more than one agent or tag (e.g., multiple).

In certain embodiments, the agent or tag is conjugated to the compound via a linker. In certain embodiments, the linker (with agent or tag) is of the formula:

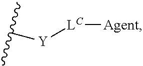

wherein Y, L^{C}, and Agent are as defined below. As described above. In certain embodiments, the linker may be attached to any position on the macrocycle (e.g., via a sugar or crosslink). In certain embodiments, the linker is attached to any one of R¹, R², R³, R^{O}, or R^{N} on any one of Formulae (I) through (I-h) described above. In certain embodiments, the linker is attached to R³. In certain embodiments, at least one instance of R³ is of the formula:

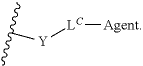

In certain embodiments, one instance of R³ is of the formula: In certain embodiments, at least one instance of R³ is of the formula:

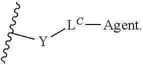

In certain embodiments,

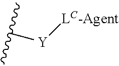

is of the formula:

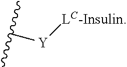

In certain embodiments,

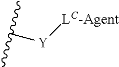

is of the formula:

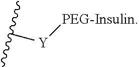

In certain embodiments,

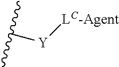

is of the formula:

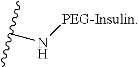

In certain embodiments, the linker For example, in certain embodiments, a conjugate provided herein is of Formula (II):

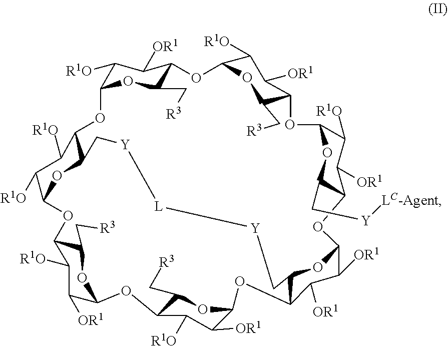

or a pharmaceutically acceptable salt, stereoisomer, or isotopically labeled derivative thereof, wherein:

L^{C} is a linker; and

Agent is an agent selected from the group consisting of proteins, peptides, nucleic acids, polysaccharides, lipids, metals, organometallic complexes, and small molecules.

In certain embodiments, the conjugate of Formula (II) is of Formula (II-a):

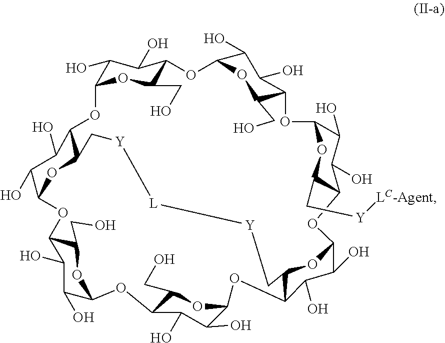

or a pharmaceutically acceptable salt, stereoisomer, or isotopically labeled derivative thereof.

As generally defined herein, “Agent” is an agent selected from the group consisting of proteins, peptides, nucleic acids, polysaccharides, lipids, metals, organometallic complexes, and small molecules. In certain embodiments, the agent is a protein. In certain embodiments, Agent is a therapeutic protein. In certain embodiments, the protein is insulin (e.g., wild-type or modified insulin). In certain embodiments, the protein is wild-type insulin (i.e., native insulin). In certain embodiments, the protein is modified insulin (i.e., an insulin analog).

As generally defined herein, L^{C} is a linker. In certain embodiments, L^{C} is a linker comprising optionally substituted alkylene, optionally substituted heteroalkylene, optionally substituted alkenylene, optionally substituted alkynylene, optionally substituted carbocyclylene, optionally substituted heterocyclylene, optionally substituted arylene, optionally substituted heteroarylene, optionally substituted acylene, —N(R^{N})—, —O—, —S—, —C(R^{C})₂ —, —C(═O)—, —C(═O)N(R^{N})—, —N(R^{N})C(═O)—, —N(R^{N})C(═O)N(R^{N})—, —C(═O)O—, —OC(═O)—, —OC(═O)O—, —OC(═O)N(R^{N})—, —N(R^{N})C(═O)O—, or any combination thereof;

wherein each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;

each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or wherein two R^{N} groups attached to the same nitrogen atom are taken together with the intervening atoms to form optionally substituted heterocyclyl or optionally substituted heteroaryl; and

each instance of R^{C} is independently hydrogen, halogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —CN, —OR^{O}, —N(R^{N})₂, or —SR^{S}; or two R^{C} are taken together to form an oxo group.

In certain embodiments, L^{C} is a polymeric linker. In certain embodiments, L^{C} is a polyethylene glycol (PEG) linker. In certain embodiments, the agent is insulin; and if is a polymeric linker. In certain embodiments, the agent is insulin; and L^{C} is a PEG linker.

Therefore, in certain embodiments, the conjugate of Formula (II) is of Formula (II-b):

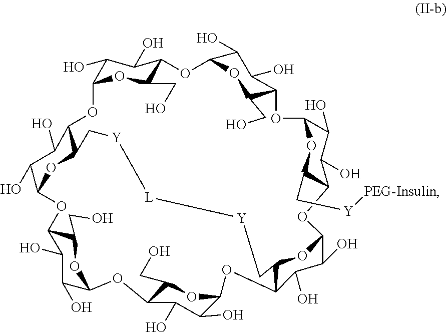

or a pharmaceutically acceptable salt, stereoisomer, or isotopically labeled derivative thereof.

In certain embodiments, the conjugate of Formula (II) is of Formula (II-c):

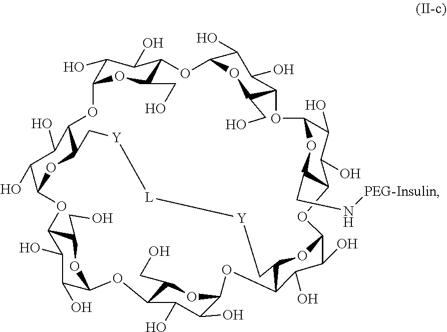

or a pharmaceutically acceptably: salt, stereoisomer, or isotopically labeled derivative thereof.

For example, in certain embodiments, the conjugate of Formula (II) of Formula (II-d):

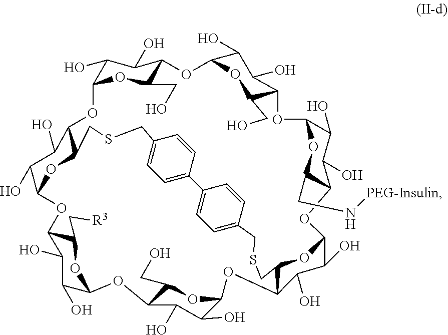

or a pharmaceutically acceptable salt thereof.

### Pharmaceutical Compositions, Kits, and Administration

The present disclosure provides pharmaceutical compositions comprising a compound or conjugate provided herein, or a pharmaceutically acceptable salt, stereoisomer, or isotopically labeled derivative thereof, and optionally a pharmaceutically acceptable excipient. In certain embodiments, the pharmaceutical composition described herein comprises a compound or conjugate provided herein, or a pharmaceutically acceptable salt, stereoisomer, or isotopically labeled derivative thereof, and a pharmaceutically acceptable excipient.

In certain embodiments, the compound or conjugate provided herein, or pharmaceutically acceptable salt, stereoisomer, or isotopically labeled derivative thereof, is provided in an effective amount in the pharmaceutical composition. In certain embodiments, the effective amount is a therapeutically effective amount. In certain embodiments, the effective amount is a prophylactically effective amount (e.g., for preventing side effects of diabetes).

Pharmaceutical compositions described herein can be prepared by any method known in the art of pharmacology. In general, such preparatory methods include bringing the compound or conjugate described herein (i.e., the “active ingredient”) into association with a carrier or excipient, and/or one or more other accessory ingredients or components, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked polyvinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, tri sodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glycervi behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, carie, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadetnia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs.

Injectable preparations, for example, sterile injectable aqueous or oleaginous suspensions can be formulated according to the known art using suitable dispersing or wetting agents and suspending agents.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art,

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required.

Suitable devices for use in delivering intradermal pharmaceutical compositions described herein include short needle devices. Intradermal compositions can be administered by devices which limit the effective penetration length of a needle into the skin.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions.

A pharmaceutical composition described herein can be prepared, packaged, and/or sold in a formulation suitable for pulmonary administration via the buccal cavity.

Pharmaceutical compositions described herein formulated for pulmonary delivery may provide the active ingredient in the form of droplets of a solution and/or suspension. Formulations described herein as being useful for pulmonary delivery are useful for intranasal delivery of a pharmaceutical composition described herein.

A pharmaceutical composition described herein can be prepared, packaged, and/or sold in a formulation for ophthalmic administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds and conjugates provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds, conjugates, and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound, conjugate, or composition required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound, conjugate, or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound, conjugate, or composition can be administered concurrently with, prior to, or subsequent to one or more additional pharmaceutical agents, which may be useful as, e.g., combination therapies. Pharmaceutical agents include therapeutically active agents. Pharmaceutical agents al so include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, anti sense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents.

Also encompassed by the disclosure are kits (e.g., pharmaceutical packs). The kits provided may comprise a pharmaceutical composition, compound, or conjugate described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound, conjugate, or pharmaceutical composition described herein. In certain embodiments, the kits are useful for treating a disease (e.g., diabetes) in a subject in need thereof. In certain embodiments, the kits are useful for preventing a disease in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., diabetes) in a subject in need thereof. In certain embodiments, the kits and instructions provide for preventing a disease in a subject in need thereof In certain embodiments, the kits and instructions provide for reducing the risk of developing a disease in a subject in need thereof. A kit described herein may include one or more additional pharmaceutical agents described herein as a separate composition.

### Methods of Treatment and Use

The present invention also provides methods of using the compounds and conjugates provided herein, or a pharmaceutically acceptable salt, stereoisomer, or isotopically labeled derivative thereof, and pharmaceutical compositions thereof.

In one aspect, the compounds, conjugates, and compositions provided herein can be used to bind glucose. Binding glucose can be important step in sensing (e.g., detecting or quantifying) glucose in vivo or in vitro. Binding glucose can also be an important step in delivering therapeutic agents (e.g., insulin) in a glucose-responsive fashion. Therefore, provided herein is a method of sensing or detecting glucose comprising contacting the glucose with a compound or conjugate provided herein, or a pharmaceutically acceptable salt, stereoisomer, or isotopically labeled derivative thereof, or a pharmaceutical composition thereof. In certain embodiments, the binding occurs in vitro. In certain embodiments, the binding occurs in vivo.

Also provided herein are compounds and conjugates provided herein, and pharmaceutically acceptable salts, stereoisomers, and isotopically labeled derivatives thereof, and pharmaceutical compositions thereof, for use in methods of sensing or detecting glucose. Also provided herein are uses of compounds and conjugates provided herein, and pharmaceutically acceptable salts, stereoisomers, and isotopically labeled derivatives thereof, and pharmaceutical compositions thereof for use in methods of sensing or detecting glucose.

In certain embodiments, the methods of sensing or detecting glucose provided herein include administering a compound or conjugate provided herein, or a pharmaceutically acceptable salt, stereoisomer, or isotopically labeled derivative thereof, or a pharmaceutical composition thereof, to a subject. In certain embodiments, the methods of sensing or detecting glucose described herein include contacting a biological sample with a compound or conjugate provided herein, or a pharmaceutically acceptable salt, stereoisomer, or isotopically labeled derivative thereof, or a pharmaceutical composition thereof.

In one aspect, the compounds, conjugates, and compositions provided herein can be used to deliver insulin to a subject (e.g., in a glucose-responsive fashion). Therefore, provided herein are methods of delivering insulin to a subject comprising administering to the subject compound or conjugate provided herein, or a pharmaceutically acceptable salt, stereoisomer, or isotopically labeled derivative thereof, or a pharmaceutical composition thereof.

Also provided herein are compounds and conjugates provided herein, and pharmaceutically acceptable salts, stereoisomers, and isotopically labeled derivatives thereof, and pharmaceutical compositions thereof, for use in methods of delivering insulin to a subject. Also provided herein are uses of compounds and conjugates provided herein, and pharmaceutically acceptable salts, stereoisomers, and isotopically labeled derivatives thereof, and pharmaceutical compositions thereof, for use in delivering insulin to a subject.

Also provided herein are methods for treating, diagnosing, or preventing a disease in a subject comprising administering to the subject a compound or conjugate provided herein, or a pharmaceutically acceptable salt, stereoisomer, or isotopically labeled derivative thereof, or a pharmaceutical composition thereof. In certain embodiment, the method is a method for treating a disease. In certain embodiments, the method is a method of diagnosing a disease (e.g., diabetes). In certain embodiments, the method is a method for preventing a disease (e.g., preventing side effects of diabetes).

In certain embodiments, the disease to be treated, diagnosed, or prevented is a genetic disease, proliferative disease (e.g., cancer), inflammatory disease, autoimmune disease, hepatic disease, splenic disease, gastrointestinal disease, pulmonary disease, hematological disease, neurological condition, painful condition, psychiatric disorder, metabolic disorder (e.g., diabetes), musculoskeletal disease, cardiovascular disease, infectious disease, or endocrine disease. In certain embodiments, the disease is a metabolic disease. In certain embodiments, the disease is diabetes. In certain embodiments, the disease is type I diabetes. In certain embodiments, the disease is type II diabetes.

Also provided herein are compounds and conjugates provided herein, and pharmaceutically acceptable salts, stereoisomers, and isotopically labeled derivatives, thereof, and pharmaceutical compositions thereof, for use in treating, diagnosing, or preventing a disease in a subject. Also provided herein are uses of compounds and conjugates provided herein, and pharmaceutically acceptable salts, stereoisomers, and isotopically labeled derivatives thereof, and pharmaceutical compositions thereof, for the manufacture of a medicament for treating, diagnosing, or preventing a disease in a subject.

In certain embodiments, the methods described herein comprise administering to a subject a therapeutically effective amount of a compound or conjugate provided herein, or a pharmaceutically acceptable salt, stereoisomer, or isotopically labeled derivative thereof, or a pharmaceutical composition thereof. In certain embodiments, the methods described herein comprise administering to a subject a prophylactically effective amount compound of a compound or conjugate provided herein, or a pharmaceutically acceptable salt, stereoisomer, or isotopically labeled derivative thereof, or a pharmaceutical composition thereof.

A compound, conjugate, or composition provided herein may be administered concurrently with, prior to, or subsequent to, one or more additional therapeutically active agents. In general, each agent will be administered at a dose and/or on a time schedule determined for that agent. It will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

In certain embodiments, the subject being treated is a mammal. In certain embodiments, the subject is a human (e.g., a human diagnosed with Type I diabetes or pre-diabetes). In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

### EXAMPLES

These and other aspects of the present invention will be further appreciated upon consideration of the following Examples, which are intended to illustrate certain particular embodiments of the invention but are not intended to limit its scope, as defined by the claims.

Cyclodextrin supramolecular scaffolds can be modified to modulate their electronic distribution and interactions. Studies have shown that various elaborate aromatic systems with polar and apolar surfaces complementing the all-equatorial substitution pattern of the substrate are able to surround glucose or other carbohydrate and show specific molecular recognition. (see, e.g., Sun, X. & James, T. D. Glucose Sensing in Supramolecular Chemistry. Chem Rev 115, 8001-8037, (2015); Klein, E., Crump, M. P. & Davis, A. P. Carbohydrate recognition in water by a tricyclic polyamide receptor. Angew Chem Int Ed Engl 44, 298-302 (2004); Ferrand, Y., Crump, M. P. & Davis, A. P. A synthetic lectin analog for biomimetic disaccharide recognition. Science 318, 619-622 (2007); Ke, C., Destecroix, H., Crump, M. P. & Davis, A. P. A simple and accessible synthetic lectin for glucose recognition and sensing. Nat Chem 4, 718-723 (2012); Mandal, P. K. et al. Crystal structure of a complex between beta-glucopyranose and a macrocyclic receptor with dendritic multicharged water solubilizing chains. Chem Commun (Camb) 52, 9355-9358 (2016)). Recently, the crystal structure of the complex between one of the potent binder equipped with dendritic multi-anionic solubilizing chains with glucose revealed rigid directed hydrogen bonding with glucose as well as extensive C—H π interactions with aromatic moieties (see, e.g., Ke, C., Destecroix, H., Crump, M. P. & Davis, A. P. A simple and accessible synthetic lectin for glucose recognition and sensing. Nat Chem 4, 718-723 (2012)).

As exemplified herein, polar hydrogen bonding chiral circular scaffolds of cyclodextrins were modified with aromatic π stacker to induce hydrogen bonding and C—H π interactions for glucose binding. The characteristics required for establishing a framework for the synthetic glucose receptor involve chiral, rigid, cage-like water soluble structure having binding pocket dimension, for example, around ˜8 Å. The library of amphiphilic cyclodextrin scaffolds based on these design principles were selected for molecular dynamics studies using the Desmond program, an explicit solvent MD package (version 3.1, Desmond Molecular Dynamics System; D. E. Shaw Research, New York, N.Y., USA and version 3.1, Maestro-Desmond Interoperability Tools; Schrödinger) with inbuilt optimized potentials for liquid simulation (OPLS 2005) force field.

Following iterative molecular dynamics (MD) screening, cyclodextrin caged structures, CDC1-CDC3 were identified displaying stabilization of glucose in the pocket for more than 25 nanoseconds. They were then synthesized. CDC1 was synthesized using thio-alkylation chemistry by coupling dibromomethylphenyl with commercially available bis-thiolated cyclodextrin under mildly basic conditions (pH 8) at room temperature and purified by HPLC.

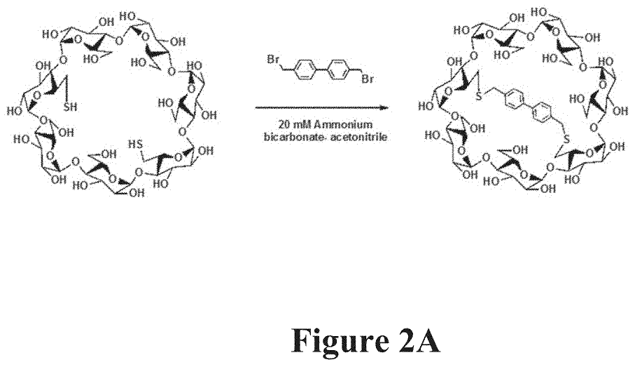

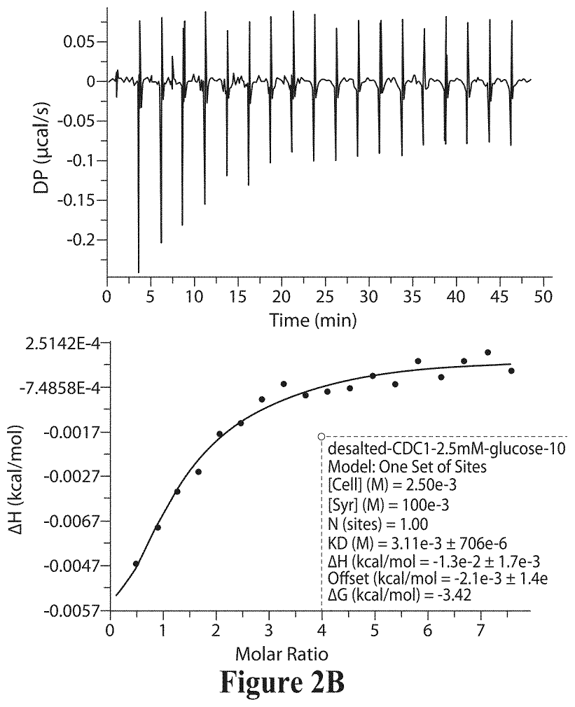

Binding studies were then carried out using isothermal titration calorimetry (ITC) to measure thermodynamic properties during binding. ITC was chosen as the preferred method to confirm binding of glucose as this assay excludes the possibilities of any false positive data. The ITC based binding assay needed significant optimization with respect to macromolecule concentration, choice of buffer, ways of desalting, concentration of the glucose solution, etc. It was observed that the binding isotherm of CDC1 showed moderate binding affinity (Kd) in the range of 5 mM (FIGS. 2A-2B). This result proves the effectiveness of MD simulation and that the cyclodextrin scaffolds can be modulated for introducing glucose binding properties.

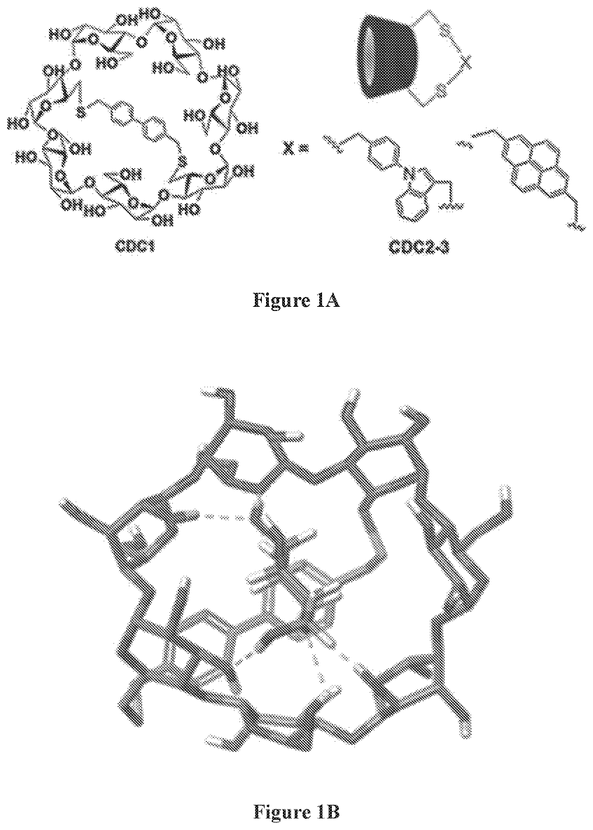

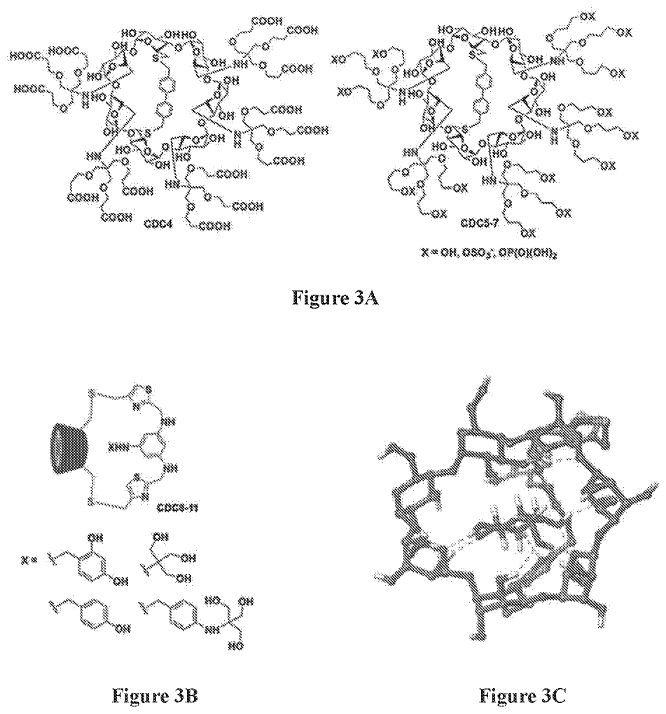

Further iterations of computational screening were carried out on cyclodextrins by substituting S bridging atom (FIGS. 1A-1B) with N and O. CDC1-CDC3 were then modified with different linkers, hydrogen bond donors, and soluhilizing groups including tricarboxylates, sulfates, and phosphates. More candidates were identified that had glucose stable in the pocket for more than 25-nanoseconds (FIG. 3A). Interestingly, one of them (CDC7) docked glucose in the pocket for more than 100 nanoseconds. Moreover, when the aromatic linker domain was modified with an amino thiazole library, more potential glucose binders were found having more than 100 nanoseconds stability in the pocket (FIG. 3B).

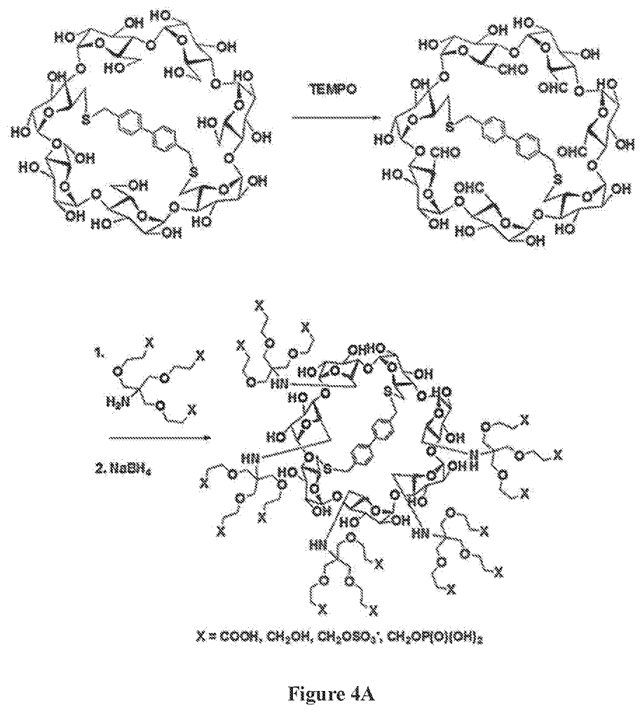

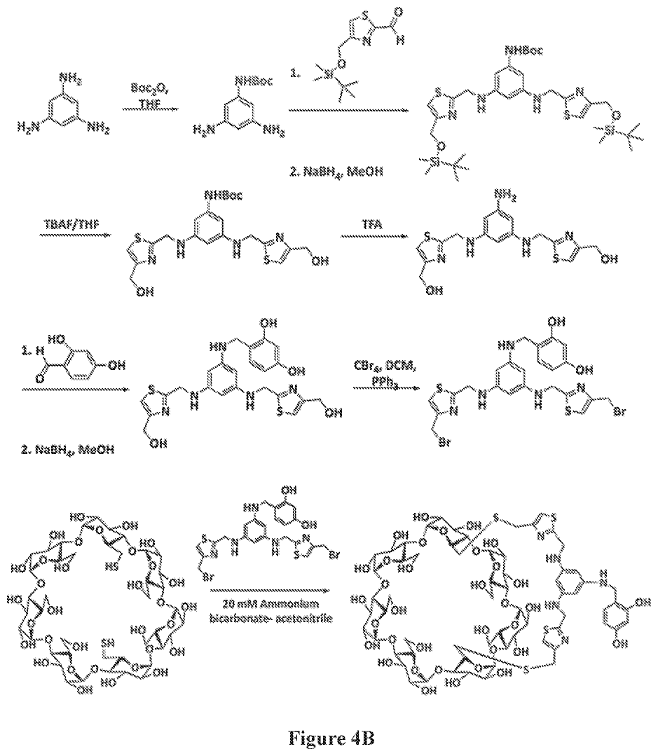

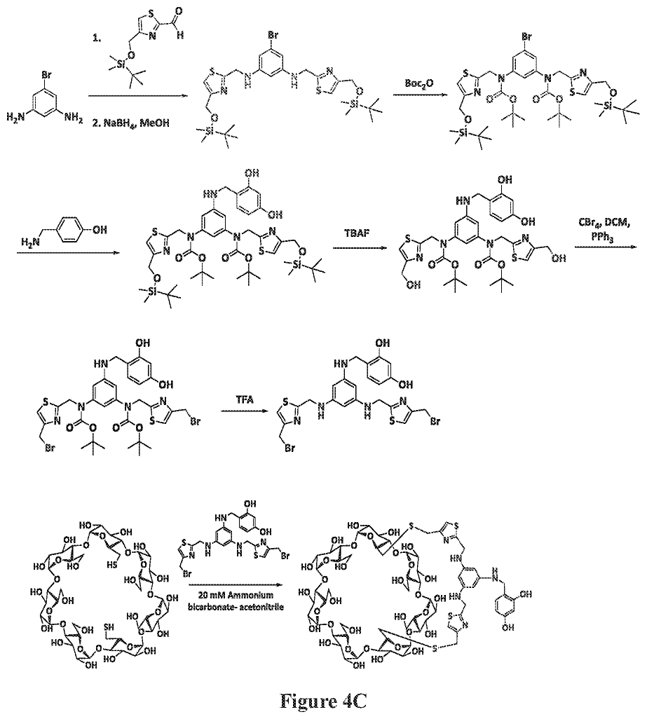

CDC4-CDC7 can be synthesized from CDC1-CDC7 in 3-4 steps, where the primary alcohol of CDC1 will be oxidized to aldehyde and subjected to reductive amination with tiicarboxylate or ttialcohol or trisubstituted alcohol linkers (FIG. 4A). For the synthesis of CDC8-11, the dibromo ring is first generated from triaminobenzene and thiazole, and coupled to bis-thiolated CD using the same thio-alkylation chemistry (FIG. 4B). All the compounds were purified by HPLC and desalted using desalting column to avoid buffer mismatch during ITC studies. The identity of compounds will be confirmed using liquid chromatography mass-spectrometry (LCMS) and Matrix Assisted Laser Desorption Ionization (MALDI). The purity of the compounds (greater than 95%) will be verified by NMR.

The biocompatible, easily synthesizable cyclodextrin-based glucose binders described herein have both hydrophobic and hydrogen bonding properties which are needed in order to stabilize glucose selectively. In this regard, the supramolecular scaffolds being highly tunable, modular, responsive, and biomimetic confers control over properties including their host-guest chemistry. The supramolecular glucose binders exemplified herein have the potential to significantly affect the insulin therapy and get translated to non-human primates testing and eventually clinical trials setting a new paradigm in translational insulin therapeutics. This enables the development of the first-time advanced glucose-responsive insulin with extreme selectivity and sensitivity to physiologically relevant glucose levels that will reduce the lag between sensing and therapeutic delivery affording long-term glucose-mediated insulin activity, enhancing the duration of insulin independence, and forming the basis for non-human primates testing and eventually clinical trials. Additionally, these binders can also be employed in the advancement of glucose sensors with long-term implantability and sensitivity with enhanced patient compliance and glucose-responsive delivery of Type 2 diabetes drugs like metformin and glipizide.

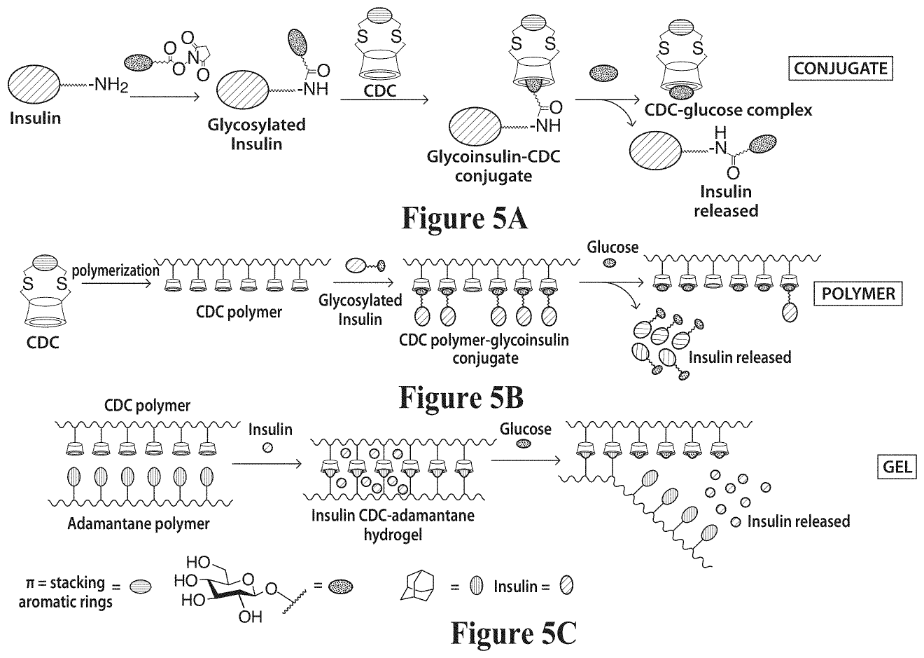

Glucose binding cyclodextrin caged structures (CDCs) can be used to modify insulin to develop glucose responsiveness. One strategy is to modify insulin with glucose using the N-Ethyl-N′-(3-dimethylaminopropyl)carbodiimide (EDC)/N-hydroxysuccinimide (NHS) coupling at the ε-amine of the B29 lysine residue of insulin (FIG. 5A). The non-covalent conjugate of modified insulin and CDC can be prepared by mixing them in 1:1 molar ratio. Secondly, CDC can be polymerized by mono-functionalizing CDC with an acrylate linker followed by Atom transfer radical polymerization (ATRP) (FIG. 5B). Here, one can check whether CDC will show multivalent interaction with high avidity that is commonly observed in case of carbohydrate binding lectins (see, e,g, Belitsky, J. M., Nelson, A., Hernandez, J. D., Baum, L. G., Stoddart, J. F. Multivalent interactions between lectins and supramolecular complexes: Galectin-1 and self-assembled pseudopolyrotaxanes. Chem Biol. 14, 1140-1151 (2007)). The conjugate of CDC polymer and glycosylated insulin (insulin aggregates) can be generated by mixing CDC units in the polymer and insulin in 1:1 molar ratio. Thirdly, a CDC based glucose-responsive insulin hydrogel can be prepared by combining CDC pendant hyaluronic acid (HA) polymer and adamantine (AD) pendant HA polymer encapsulating insulin. AD will have lower binding affinity to the modified CDC (as compared to cyclodextrin, which has a dissociation constant in micromolar range) and will get competitively removed by glucose from the CDC pocket. The encapsulated insulin can act as a glucose-responsive drug depot that will release insulin on exposure to glucose (FIG. 5C).

### EQUIVALENTS AND SCOPE

Text here. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound, or a pharmaceutically acceptable salt thereof, comprising a cyclodextrin, wherein two sugars of the cyclodextrin are connected via a linker; and the linker comprises one or more aromatic rings.

2. The compound of claim 1, or a pharmaceutically acceptable salt thereof, wherein the cyclodextrin is an α-cyclodextrin, β-cyclodextrin, or γ-cyclodextrin.

3. The compound of claim 2, or a pharmaceutically acceptable salt thereof, wherein the cyclodextrin is a β-cyclodextrin.

4. The compound of any one of claim 1-3, or a pharmaceutically acceptable salt thereof, wherein the sugars connected via a linker are separated by 1, 2, or 3 sugars.

5. The compound of claim 4, or a pharmaceutically acceptable salt thereof, wherein the sugars connected via a linker are separated by 2 sugars.

6. The compound of any one of claims 1-5, or a pharmaceutically acceptable salt thereof, wherein the linker is of the formula: 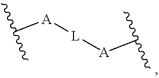
wherein:
L is optionally substituted alkylene, optionally substituted heteroalkylene, optionally substituted alkenylene, optionally substituted alkynylene, optionally substituted carbocyclylene, optionally substituted heterocyclylene, optionally substituted arylene, optionally substituted heteroarylene, optionally substituted acylene, —N(R^{N})—, —O—, —S—, —C(R^{C})₂—, —C(═O)—, —C(═O)N(R^{N})—, —N(R^{N})C(═O)—, —N(R^{N})C(═O)N(R^{N})—, —C(═O)O—, —OC(═O)—, —OC(═O)O—, —OC(═O)N(R^{N})—, —N(R^{N})C(═O)O—, or any combination thereof;
provided that L comprises one or more moieties selected from the group consisting of optionally substituted aryl, optionally substituted arylene, optionally substituted heteroaryl, optionally substituted heteroarylene, optionally substituted arylalkyl, and optionally substituted heteroarylalkyl; and
each instance of A is independently a bond, optionally substituted alkylene, optionally substituted heteroalkylene, —O—, —N(R^{N})—, —S—, —C(R^{C})₂—, —C(═O)—, —C(R^{C})₂O—, —C(R^{C})₂N(R^{N})—, or —C(R^{C})₂S—;
each instance of is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;
each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or wherein two R^{N} groups attached to the same nitrogen atom are taken together with the intervening atoms to form optionally substituted heterocyclyl or optionally substituted heteroaryl;
each instance of R^{C} is independently hydrogen, halogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —CN, —OR^{O}, —N(R^{N})₂, or —SR^{S}; or two R^{C} are taken together to form an oxo group.

7. The compound of any one of claims 1-5, or a pharmaceutically acceptable salt thereof, wherein the linker is of the formula: 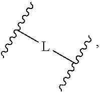
wherein:
L is optionally substituted alkylene, optionally substituted heteroalkylene, optionally substituted alkenylene, optionally substituted alkynylene, optionally substituted carbocyclylene, optionally substituted heterocyclylene, optionally substituted arylene, optionally substituted heteroarylene, optionally substituted acylene, —N(R^{N})—, —O—, —S—, —C(R^{C})₂—, —C(═O)—, —C(═O)N(R^{N})—, —N(R^{N})C(═O)—, —N(R^{N})C(═O)N(R^{N})—, —C(═O)O—, —OC(═O)—, —OC(═O)—, —OC(═O)N(R^{N})—, —N(R^{N})C(═O)O—, or any combination thereof;
provided that L comprises one or more moieties selected from the group consisting of optionally substituted aryl, optionally substituted arylene, optionally substituted heteroaryl, optionally substituted heteroarylene, optionally substituted arylalkyl, and optionally substituted heteroarylalkyl; and
each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;
each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or wherein two R^{N} groups attached to the same nitrogen atom are taken together with the intervening atoms to form optionally substituted heterocyclyl or optionally substituted heteroaryl;
each instance of R^{C} is independently hydrogen, halogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —CN, —OR^{O}, —N(R^{N})₂, or —SR^{S}; or two R^{C} are taken together to form an oxo group.

8. A compound of Formula (I): 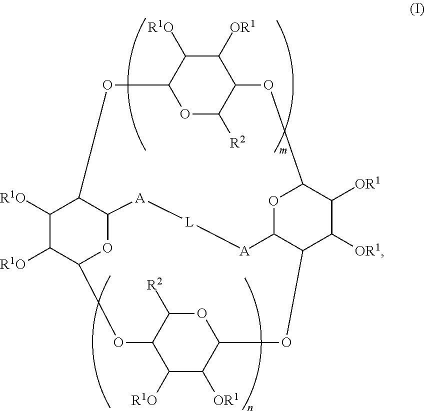
or a pharmaceutically acceptable salt thereof, wherein:
m is an integer between 1 and 10, inclusive;
n is an integer between 1 and 10, inclusive;
L is a linker comprising optionally substituted alkylene, optionally substituted heteroalkylene, optionally substituted alkenylene, optionally substituted alkynylene, optionally substituted carbocyclylene, optionally substituted heterocyclylene, optionally substituted arylene, optionally substituted heteroarylene, optionally substituted acylene, —N(R^{N})—, —O—, —S—, —C(R^{C})₂—, —C(═O)—, —C(═O)N(R^{N})—, —N(R^{N})C(═O)—, —N(R^{N})C(═O)N(R^{N})—, —C(═O)O—, —OC(═O)—, —OC(═O)O—, —OC(═O)N(R^{N})—, —N(R^{N})C(═O)O—, or any combination thereof;
provided that L comprises one or more moieties selected from the group consisting of optionally substituted aryl, optionally substituted arylene, optionally substituted heteroaryl, optionally substituted heteroarylene, optionally substituted arylalkyl, and optionally substituted heteroarylalkyl;
each instance of A is independently a bond, optionally substituted alkylene, optionally substituted heteroalkylene, —O—, —N(R^{N})—, —S—, —C(R^{C})₂—, —C(═O)—, —C(R^{C})₂O—, —C(R^{C})₂N(R^{N})—, or —C(R^{C})₂S—;
each instance of R¹ is independently hydrogen, optionally substituted alkyl, optionally substituted carbocyclyl, optionally substituted heterocyclyl, optionally substituted aryl, optionally substituted heteroaryl, optionally substituted optionally substituted acyl, an oxygen protecting group, or a sugar;
each instance of R² is independently optionally substituted alkyl, optionally substituted heteroalkyl, optionally substituted carbocyclyl, optionally substituted heterocyclyl, optionally substituted aryl, optionally substituted heteroaryl, optionally substituted acyl, —OR^{O}, —N(R^{N})₂, —SR^{S}, —C(R^{C})₂OR^{O}, —C(R^{C})₂N(R^{N})₂, —C(R^{C})₂SR^{S}, or a sugar;
each instance of R^{O} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or an oxygen protecting group;
each instance of R^{N} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a nitrogen protecting group; or wherein two R^{N} groups attached to the same nitrogen atom are taken together with the intervening atoms to form optionally substituted heterocyclyl or optionally substituted heteroaryl;
each instance of R^{S} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, or a sulfur protecting group; and
each instance of R^{C} is independently hydrogen, halogen, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted acyl, —CN, —OR^{O}, —N(R^{N})₂, or —SR^{S}; or two R^{C} are taken together to form an oxo group.

9. The compound of claim 8, wherein the compound is of Formula (I-a): 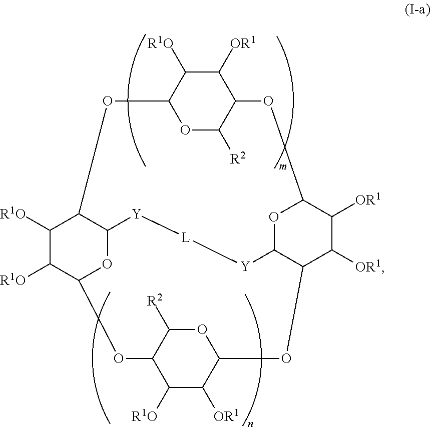
or a pharmaceutically acceptable salt thereof, wherein:
each instance of Y is independently a bond, —C(R^{C})₂—, —O—, —S—, or —N(R^{N})—.

10. The compound of claim 8 or 9, wherein the compound is of Formula (I-b): 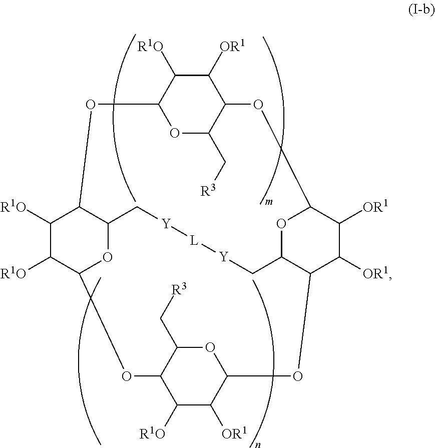
or a pharmaceutically acceptable salt thereof, wherein:
each instance of R³ is independently —OR^{O}, —N(R^{N})₂, or —SR^{S}.

11. The compound of any one of claims 8-10, wherein the compound is of Formula I-c : 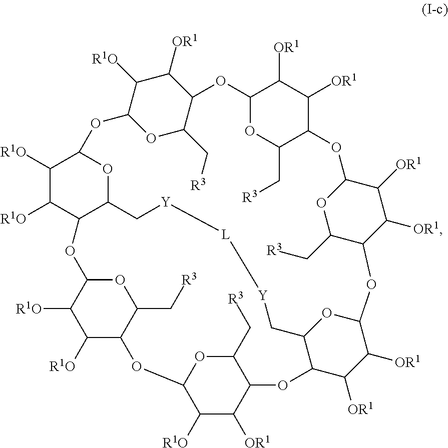
or a pharmaceutically acceptable salt thereof.

12. The compound of any one of claims 8-11, wherein the compound is of Formula (I-d): 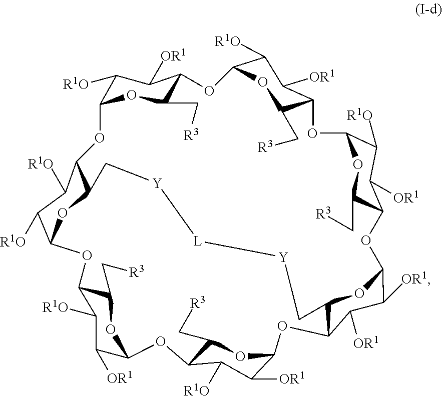
or a pharmaceutically acceptable salt thereof.

13. The compound of any one of claims 8-12, wherein the compound is of Formula (I-e): 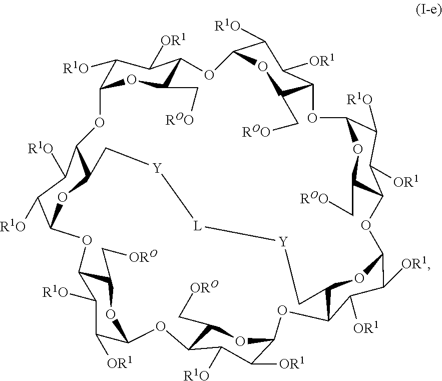
or a pharmaceutically acceptable salt thereof.

14. The compound of any one of claims 8-13, wherein the compound is of Formula (I-f): 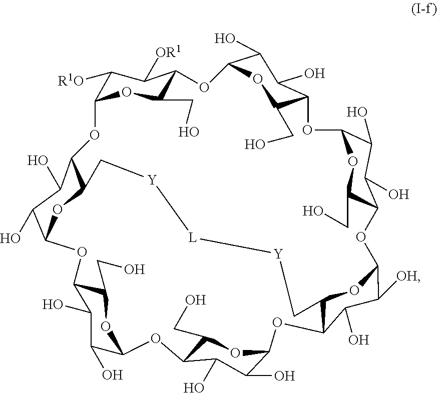
or a pharmaceutically acceptable salt thereof.

15. The compound of any one of claims 8-13, wherein the compound is of Formula (I-g): 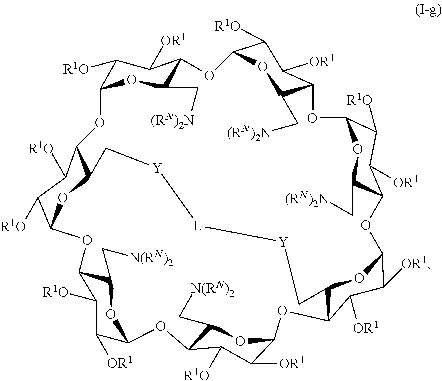
or a pharmaceutically acceptable salt thereof.

16. The compound of any one of claims 8-13 and 15, wherein the compound is of Formula (I-h): 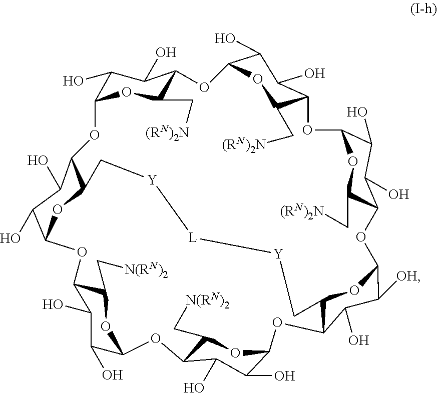
or a pharmaceutically acceptable salt thereof.

17. The compound of any one of claims 8-10, or a pharmaceutically acceptable salt thereof, wherein m is an integer from 1-5, inclusive.

18. The compound of claim 17, or a pharmaceutically acceptable salt thereof, wherein m is 3.

19. The compound of any one of claims 8-10, 17 and 18, or a pharmaceutically acceptable salt thereof wherein n is an integer from 1-5, inclusive.

20. The compound of claim 19, or a pharmaceutically acceptable salt thereof wherein n is 2.

21. The compound of any one of claims 8-10 and 17-20, or a pharmaceutically acceptable salt thereof, wherein the sum of m and n is 4.

22. The compound of any one of claims 8-10 and 17-20, or a pharmaceutically acceptable salt thereof wherein the sum of m and n is 5.

23. The compound of any one of claims 8-10 and 17-20, or a pharmaceutically acceptable salt thereof, wherein the sum of m and n is 6.

24. The compound of any one of claims 6, 8, and 17-23, or a pharmaceutically acceptable salt thereof, wherein each instance of A is independently optionally substituted C₁₋₃ heteroalkylene.

25. The compound of claim 24, or a pharmaceutically acceptable salt thereof, wherein each instance of A is independently unsubstituted C₁₋₃ heteroalkylene.

26. The compound of claim 24 or 25, or a pharmaceutically acceptable salt thereof, wherein each instance of A is independently —C(R^{C})₂O—, —C(R^{C})₂N(R^{N})—, or —C(R^{C})₂S—.

27. The compound of any one of claims 24-26, or a pharmaceutically acceptable salt thereof, wherein each instance of A is independently —CH₂O—, —CH₂S—, or —CH₂N(R^{N})—.

28. The compound of claim 27, or a pharmaceutically acceptable salt thereof, wherein each instance of A is —CH₂O—.

29. The compound of claim 27, or a pharmaceutically acceptable salt thereof, wherein each instance of A is —CH₂S—.

30. The compound of claim 27, or a pharmaceutically acceptable salt thereof, wherein each instance of A is —CH₂NH—.

31. The compound of any one of claims 9-23, or a pharmaceutically acceptable salt thereof, wherein each instance of Y is independently —O—, —S—, or —N(R^{N})—.

32. The compound of claim 31, or a pharmaceutically acceptable salt thereof, wherein each instance of Y is —O—.

33. The compound of claim 31, or a pharmaceutically acceptable salt thereof, wherein each instance of Y is —S—.

34. The compound of claim 31, or a pharmaceutically acceptable salt thereof, wherein each instance of Y is —NH—.

35. The compound of any one of claims 6-34, or a pharmaceutically acceptable salt thereof, wherein L comprises 1-200 non-hydrogen atoms.

36. The compound of claim 35, or a pharmaceutically acceptablesalt thereof, wherein L comprises 1-100 non-hydrogen atoms.

37. The compound of claim 35, or a pharmaceutically acceptable salt thereof, wherein L comprises 1-50 non-hydrogen atoms.

38. The compound of any one of claims 6-37, or a pharmaceutically acceptable salt thereof, wherein L comprises one or more optionally substituted aryl or optionally substituted arylene moieties.

39. The compound of claim 38, or a pharmaceutically acceptable salt thereof, wherein L comprises one or more optionally substituted C₆-C₂₀ aryl or optionally substituted C₆-C₂₀ arylene moieties.

40. The compound of claim 38, or a pharmaceutically acceptable salt thereof, wherein L comprises one or more moieties selected from the group consisting of optionally substituted phenyl, optionally substituted phenylene, optionally substituted naphthalenyl, optionally substituted naphthalenylene, optionally substituted anthracenyl, optionally substituted anthracenylene, optionally substituted phenanthrenyl, optionally substituted phenanthrenylene, optionally substituted tetracenyl, optionally substituted tetracenylene, optionally substituted chrysenyl, optionally substituted chrysenylene, optionally substituted pyrenyl, optionally substituted pyrenylene, optionally substituted triphenylenyl, optionally substituted triphenylenylene, optionally substituted pentacenyl, optionally substituted pentacenylene, optionally substituted benzo[a]pyrenyl, and optionally substituted benzo[a]pyrenylene.

41. The compound of any one of claims 38-40, or a pharmaceutically acceptable salt thereof, wherein L comprises one or more moieties selected from the group consisting of: 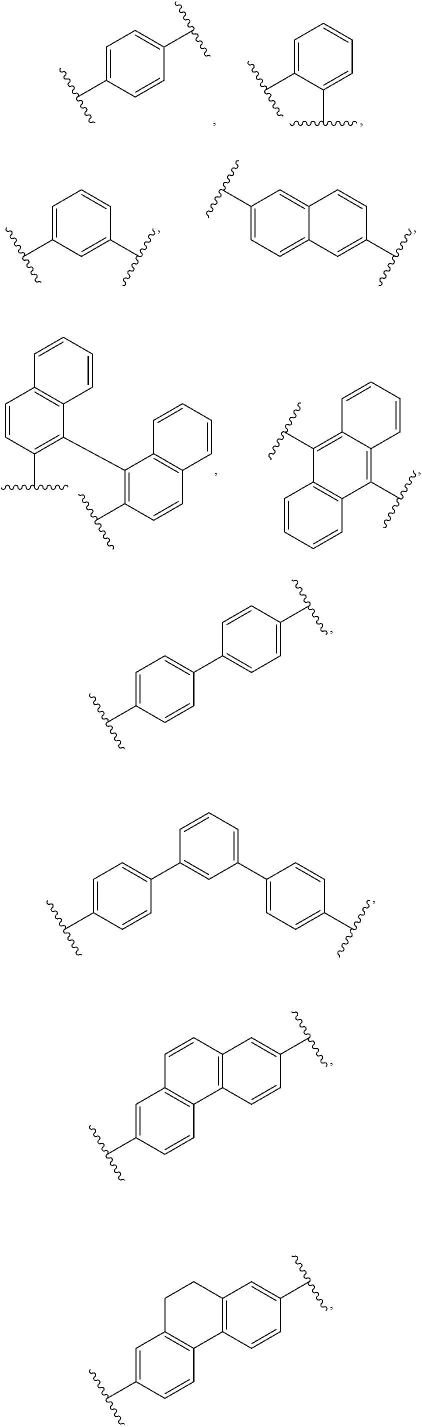 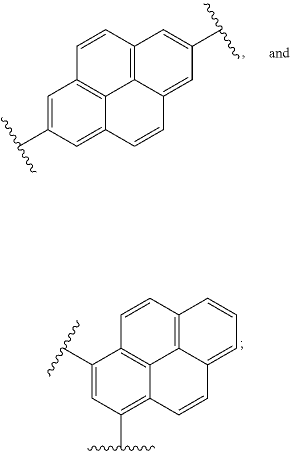 [SMILES: *c1cc2ccc3cc(*)cc4ccc(c1)c2c34 ; *c1cc(*)c2ccc3cccc4ccc1c2c43]
wherein the one or more moieties are optionally substituted.

42. The compound of any one of claims 38-41, or a pharmaceutically acceptable salt thereof, wherein L comprises one or more moieties selected from the group consisting of:

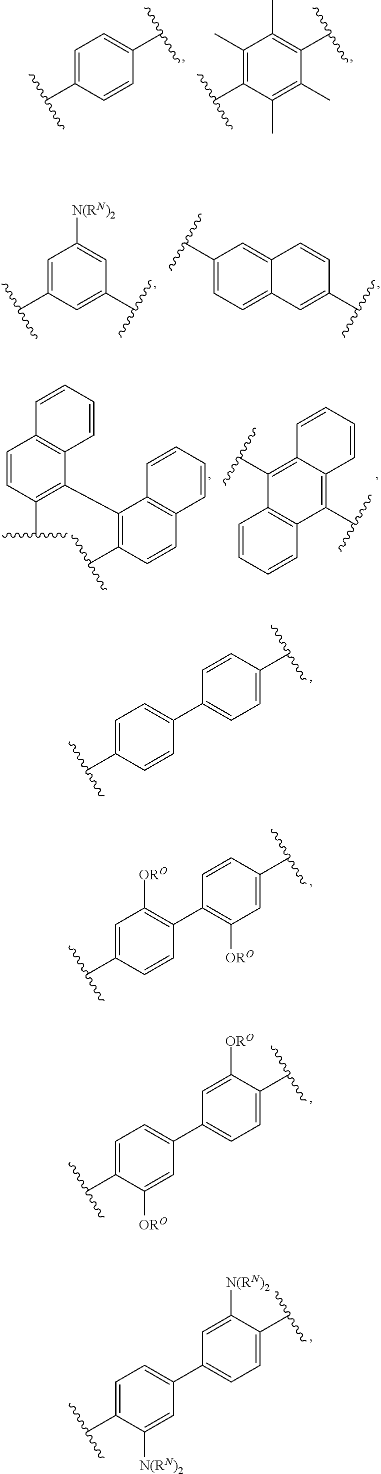

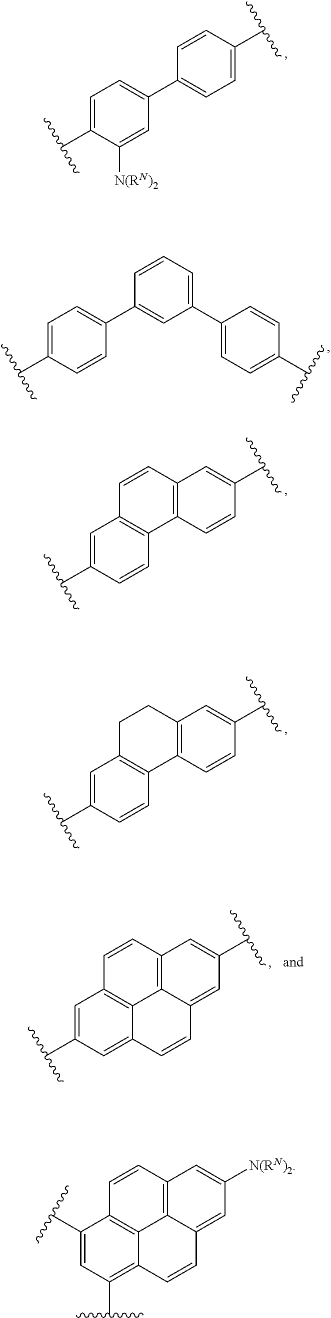

43. The compound of any one of claims 6-42, or a pharmaceutically acceptable salt thereof, wherein L comprises one or more optionally substituted heteroaryl or optionally substituted heteroarylene moieties.

44. The compound of claim 43, or a pharmaceutically acceptable salt thereof, wherein L comprises one or more optionally substituted 5-6 membered heteroaryl or optionally substituted 5-6 membered heteroarvlene moieties.

45. The compound of claim 43, or a pharmaceutically acceptable salt thereof, wherein L comprises one or more moieties selected from the group consisting of optionally substituted oxazolyl, optionally substituted oxazolylene, optionally substituted thiazolyl, optionally substituted thiazolylene, optionally substituted imidazolyl, and optionally substituted imidazolylene.

46. The compound of any one of claims 43-45, or a pharmaceutically acceptable salt thereof, wherein L comprises one or more of the following moieties:

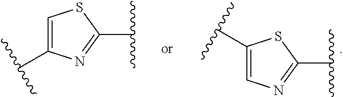 [SMILES: *c1csc(*)n1 ; *c1cnc(*)s1]

47. The compound of any one of claims 6-46, wherein L comprises one or more optionally substituted arylalkyl or optionally substituted heteroarylalkyl moieties.

48. The compound of claim 47, or a pharmaceutically acceptable salt thereof, wherein L comprises one or more optionally substituted C₃₋₂₀ arylalkyl or optionally substituted C₃₋₂₀ heteroarylalkyl moieties.

49. The compound of claim 47 or 48, or a pharmaceutically acceptable salt thereof, wherein L comprises one or more optionally substituted benzyl moieties.

50. The compound of any one of claims 6-49, or a pharmaceutica acce able salt thereof, wherein L comprises one or more amino acids.

51. The compound of claim 50, or a pharmaceutically acceptable salt thereof, wherein L comprises one or more amino acids selected from the group consisting of arginine, histidine, tryptophan, aspartic acid, glutamine, asparagine, glutamine, phenylalanine, and tryptophan.

52. The compound of claim 50 or 51, or a pharmaceutically acceptable salt thereof, wherein L comprises one or more amino acids selected from the group consisting of histidine, tryptophan, and phenylalanine.

53. The compound of any one of claims 6-52, or a pharmaceutically acceptable salt thereof, wherein L is a peptidic linker.

54. The compound of any one of claims 6-53, wherein L comprises one of the following formulae: 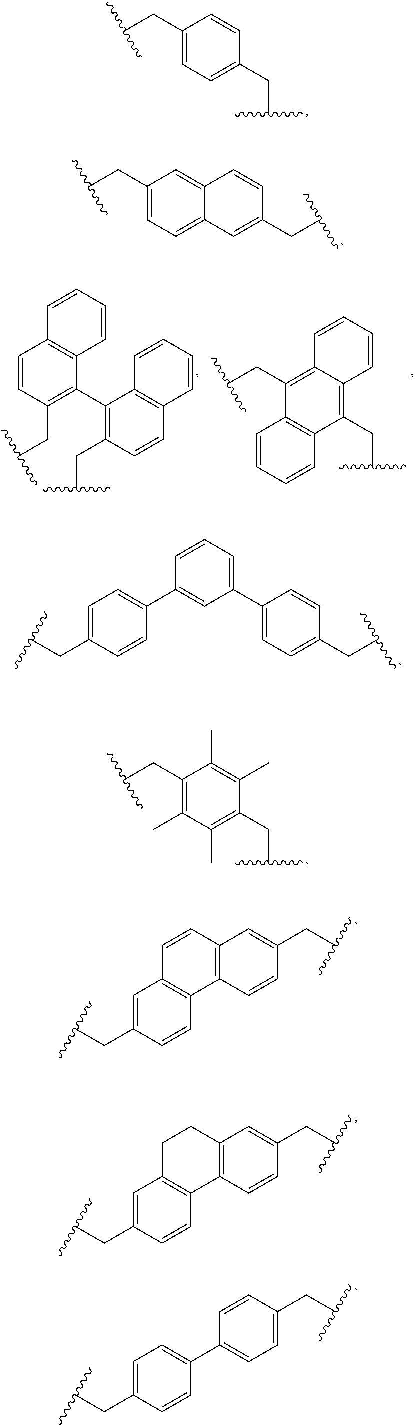 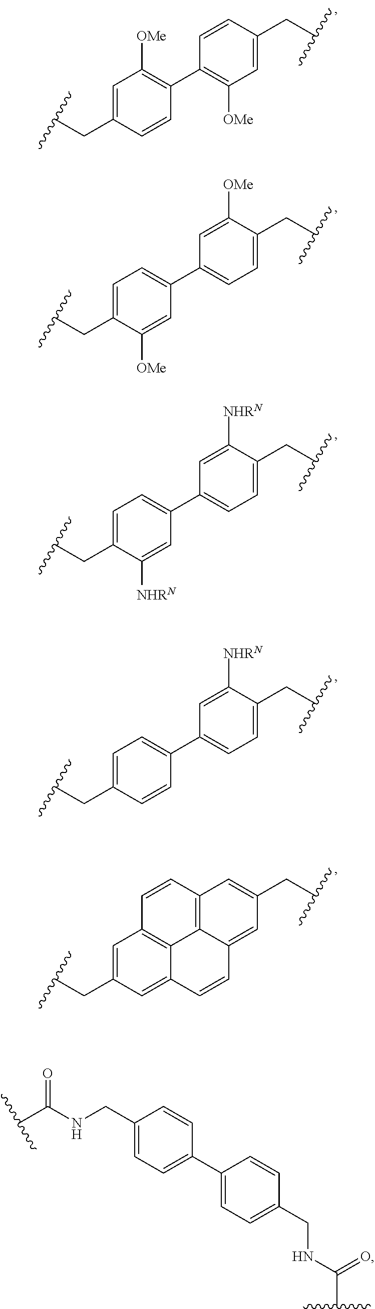 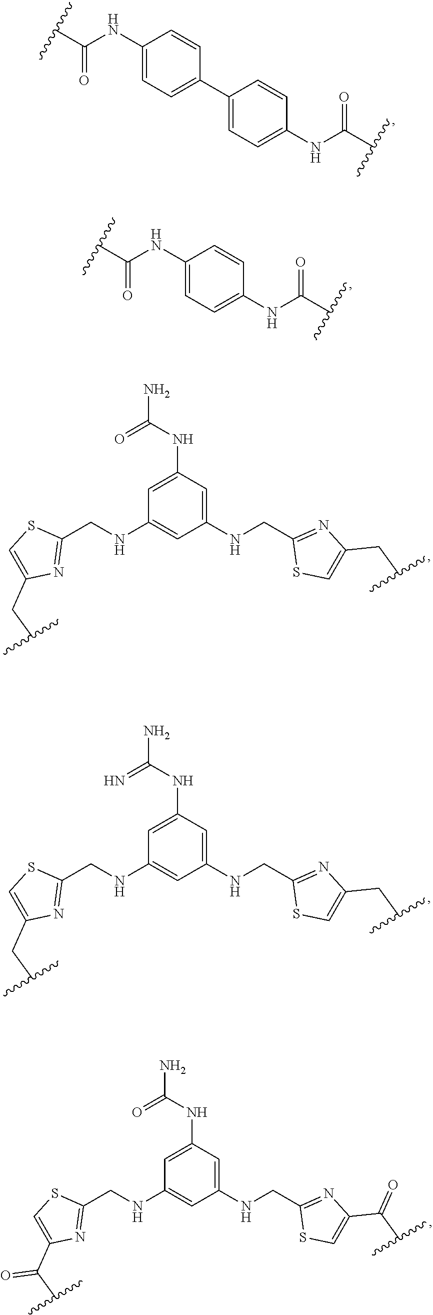 [SMILES: *C(=O)Nc1ccc(-c2ccc(NC(*)=O)cc2)cc1 ; *C(=O)Nc1ccc(NC(*)=O)cc1 ; *Cc1csc(CNc2cc(NCc3nc(C*)cs3)cc(NC(N)=O)c2)n1 ; *Cc1csc(CNc2cc(NCc3nc(C*)cs3)cc(NC(=N)N)c2)n1 ; *C(=O)c1csc(CNc2cc(NCc3nc(C(*)=O)cs3)cc(NC(N)=O)c2)n1] 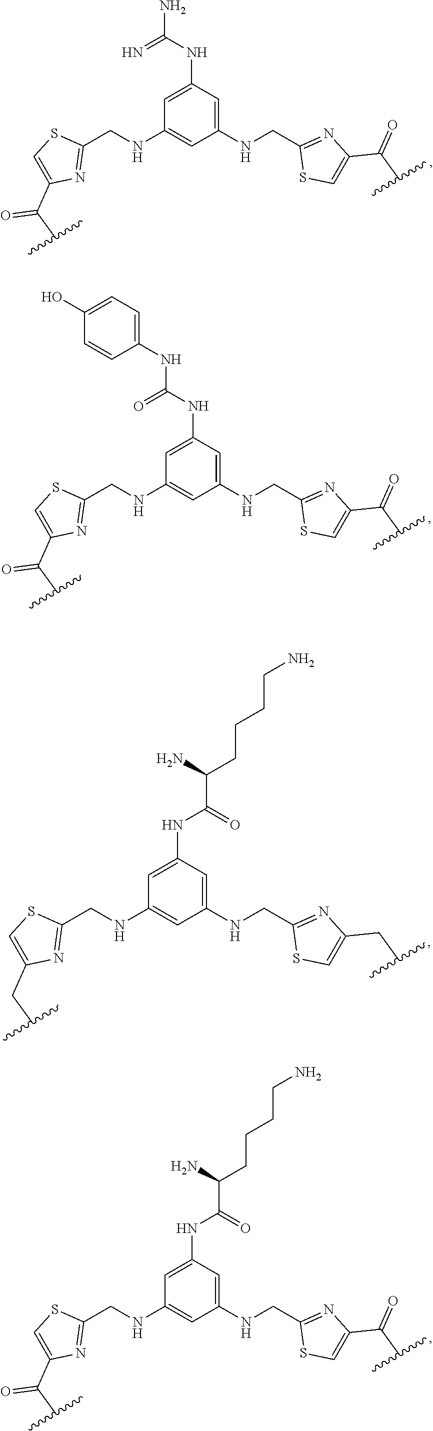 [SMILES: *C(=O)c1csc(CNc2cc(NCc3nc(C(*)=O)cs3)cc(NC(=N)N)c2)n1 ; *C(=O)c1csc(CNc2cc(NCc3nc(C(*)=O)cs3)cc(NC(=O)Nc3ccc(O)cc3)c2)n1 ; *Cc1csc(CNc2cc(NCc3nc(C*)cs3)cc(NC(=O)[C@@H](N)CCCCN)c2)n1 ; *C(=O)c1csc(CNc2cc(NCc3nc(C(*)=O)cs3)cc(NC(=O)[C@@H](N)CCCCN)c2)n1] 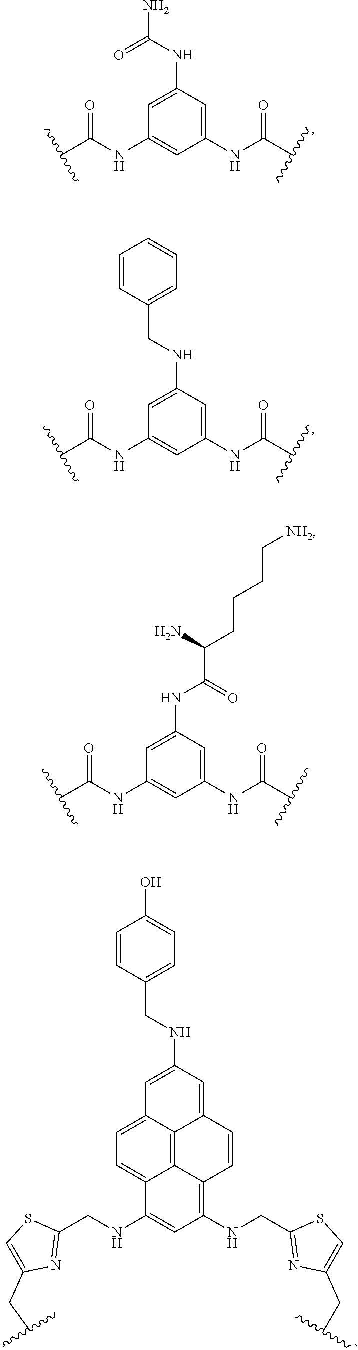 [SMILES: *C(=O)Nc1cc(NC(*)=O)cc(NC(N)=O)c1 ; *C(=O)Nc1cc(NCc2ccccc2)cc(NC(*)=O)c1 ; *C(=O)Nc1cc(NC(*)=O)cc(NC(=O)[C@@H](N)CCCCN)c1 ; *Cc1csc(CNc2cc(NCc3nc(C*)cs3)c3ccc4cc(NCc5ccc(O)cc5)cc5ccc2c3c54)n1] 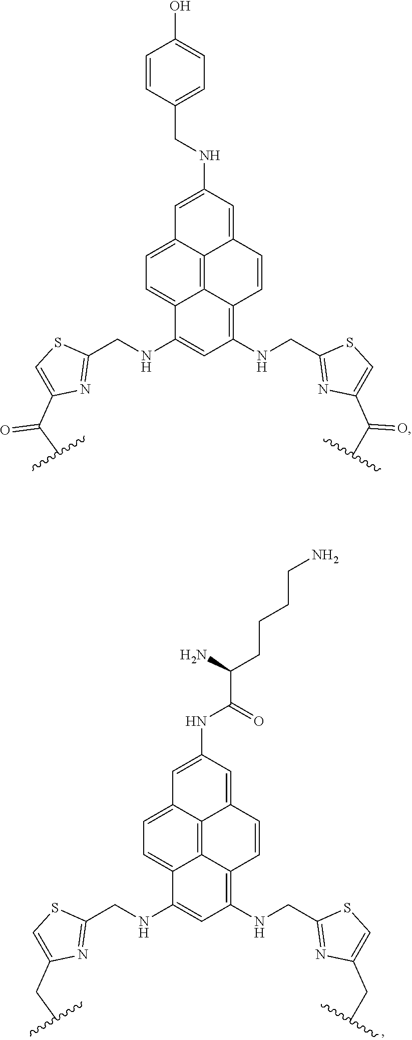 [SMILES: *C(=O)c1csc(CNc2cc(NCc3nc(C(*)=O)cs3)c3ccc4cc(NCc5ccc(O)cc5)cc5ccc2c3c54)n1 ; *Cc1csc(CNc2cc(NCc3nc(C*)cs3)c3ccc4cc(NC(=O)[C@@H](N)CCCCN)cc5ccc2c3c54)n1] 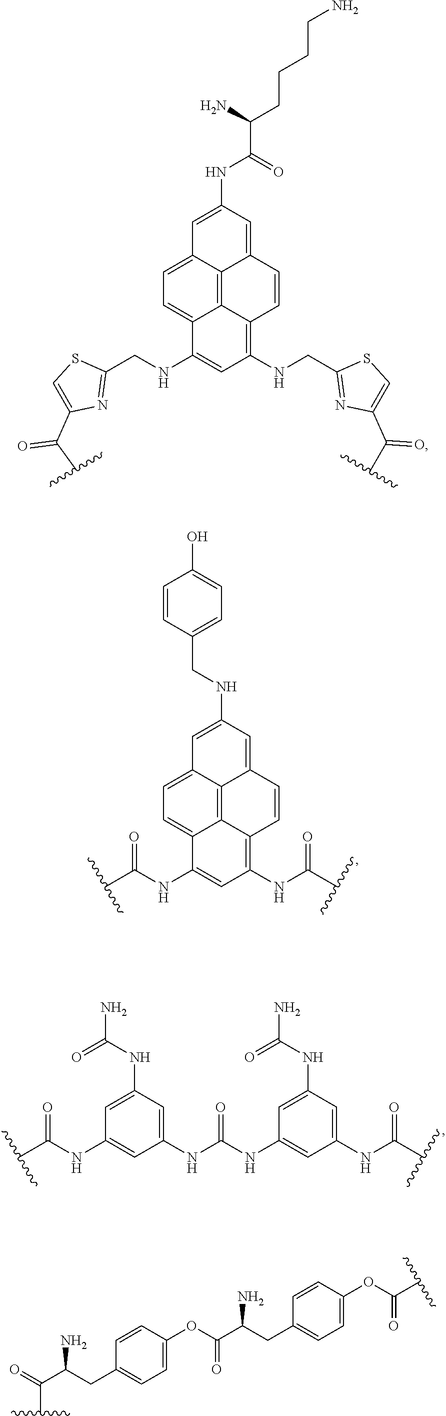 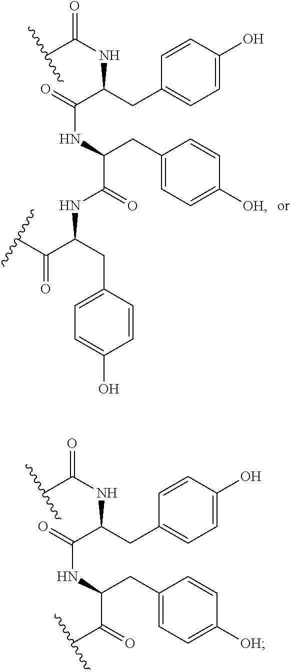 [SMILES: *C(=O)N[C@@H](Cc1ccc(O)cc1)C(=O)N[C@@H](Cc1ccc(O)cc1)C(=O)N[C@@H](Cc1ccc(O)cc1)C(*)=O ; "or" ; *C(=O)N[C@@H](Cc1ccc(O)cc1)C(=O)N[C@@H](Cc1ccc(O)cc1)C(*)=O]
wherein each instance of R^{N} is independently hydrogen or a group of one of the following formulae:

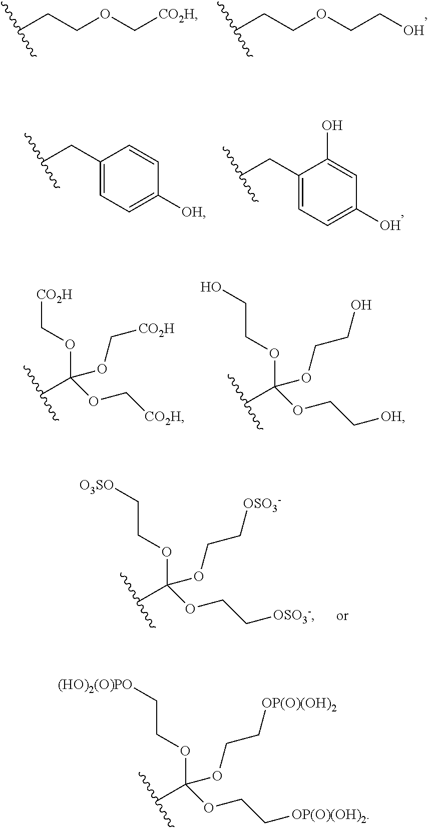

55. The compound of any one of claims 6-54, is one of the formula recited in claim 54.

56. The compound of any one of claims 8-13, 15, and 17-55, or a pharmaceutically acceptable salt thereof, wherein at least one instance of R¹ is hydrogen.

57. The compound of claim 56, or a pharmaceutically acceptable salt thereof, wherein each instance of R¹ is hydrogen.

58. The compound of any one of claims 8, 9, and 17-55, or a pharmaceutically acceptable salt thereof, wherein at least one instance of R² is —CH₂OR^{O}.

59. The compound of claim 58, or a pharmaceutically acceptable salt thereof, wherein each instance of R² is —CH₂OR^{O}.

60. The compound of claim 59, or a pharmaceutically acceptable salt thereof, wherein each instance of R² is —CH₂OH.

61. The compound of any one of claims 8, 9, and 17-55, or a pharmaceutically acceptable salt thereof, wherein at least one instance of R² is —CH₂N(R^{N})₂.

62. The compound of claim 61, or a pharmaceutically acceptable salt thereof, wherein each instance of R² is —CH₂N(R^{N})₂.

63. The compound of claim 62, or a pharmaceutically acceptable salt thereof, wherein each instance of R² is —CH₂NH₂.

64. The compound of any one of claims 10-12 and 17-57, or a pharmaceutically acceptable salt thereof, wherein at least one instance of R³ is —OR^{O}.

65. The compound of claim 64, or a pharmaceutically acceptable salt thereof, wherein each instance of R³ is —OR^{O}.

66. The compound of claim 65, wherein each instance of R³ is —OH.

67. The compound of any one of claims 10-12 and 17-57, or a pharmaceutically acceptable salt thereof, wherein at least one instance of R³ is —N(R^{N})₂.

68. The compound of claim 67, or a pharmaceutically acceptable salt thereof, wherein each instance of R³ is —N(R^{N})₂.

69. The compound of claim 68, or a pharmaceutically acceptable salt thereof, wherein each instance of R³ is —NH₂.

70. The compound of any one of claims 6-69, or a pharmaceutically acceptably: salt thereof, wherein at least one instance of R^{O} is hydrogen.

71. The compound of claim 70, or a pharmaceutically acceptable salt thereof, wherein each instance of R^{O} is hydrogen.

72. The compound of any one of claims 6-71, or a pharmaceutically acceptable salt thereof, wherein at least one instance of R^{N} is hydrogen.

73. The compound of claim 72, or a pharmaceutically acceptable salt thereof, wherein each instance of R^{N} is hydrogen.

74. The compound of any one of claims 6-71, or a pharmaceutically acceptable salt thereof, wherein at least one instance of R^{N} is optionally substituted C₁₋₆ alkyl.

75. The compound of any one of claims 6-74, or a pharmaceutica acce able salt thereof, wherein at least one instance of R^{S} is hydrogen.

76. The compound of claim 75, or a pharmaceutically acceptable salt thereof, wherein each instance of R^{S} is hydrogen.

77. The compound of any one of claims 6-76, or a pharmaceutically acceptable: salt thereof, wherein at least one instance of R^{C} is hydrogen.

78. The compound of claim 77, or a pharmaceutically acceptable salt thereof, wherein each instance of R^{C} is hydrogen.

79. The compound of any one of the preceding claims, or a pharmaceutically acceptable salt thereof, wherein the compound is selected from the group consisting of: 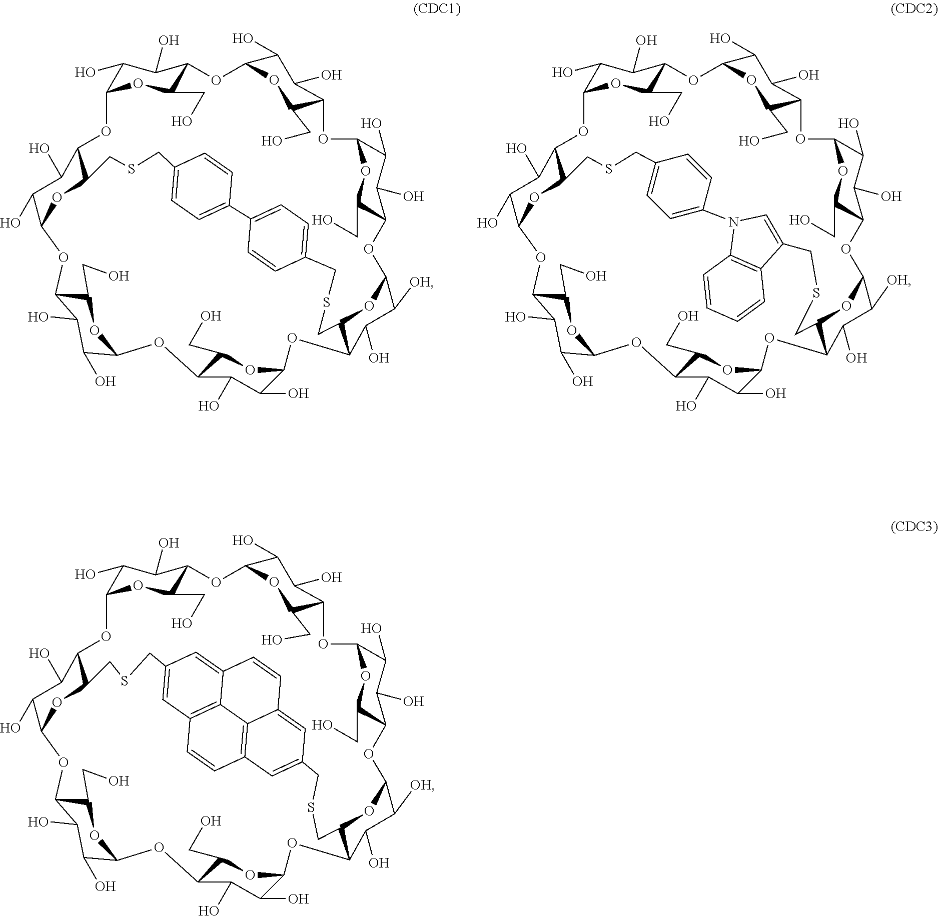 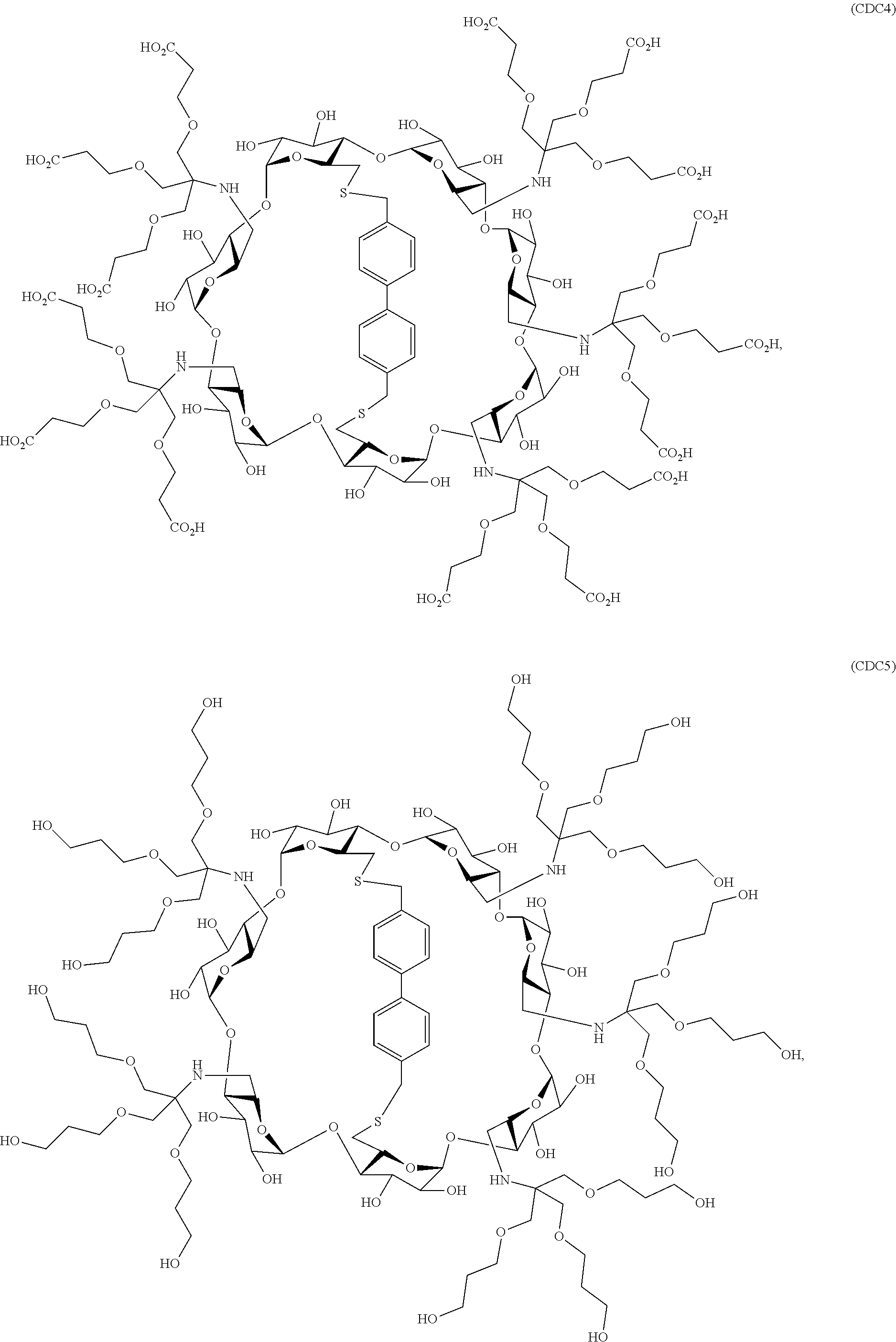 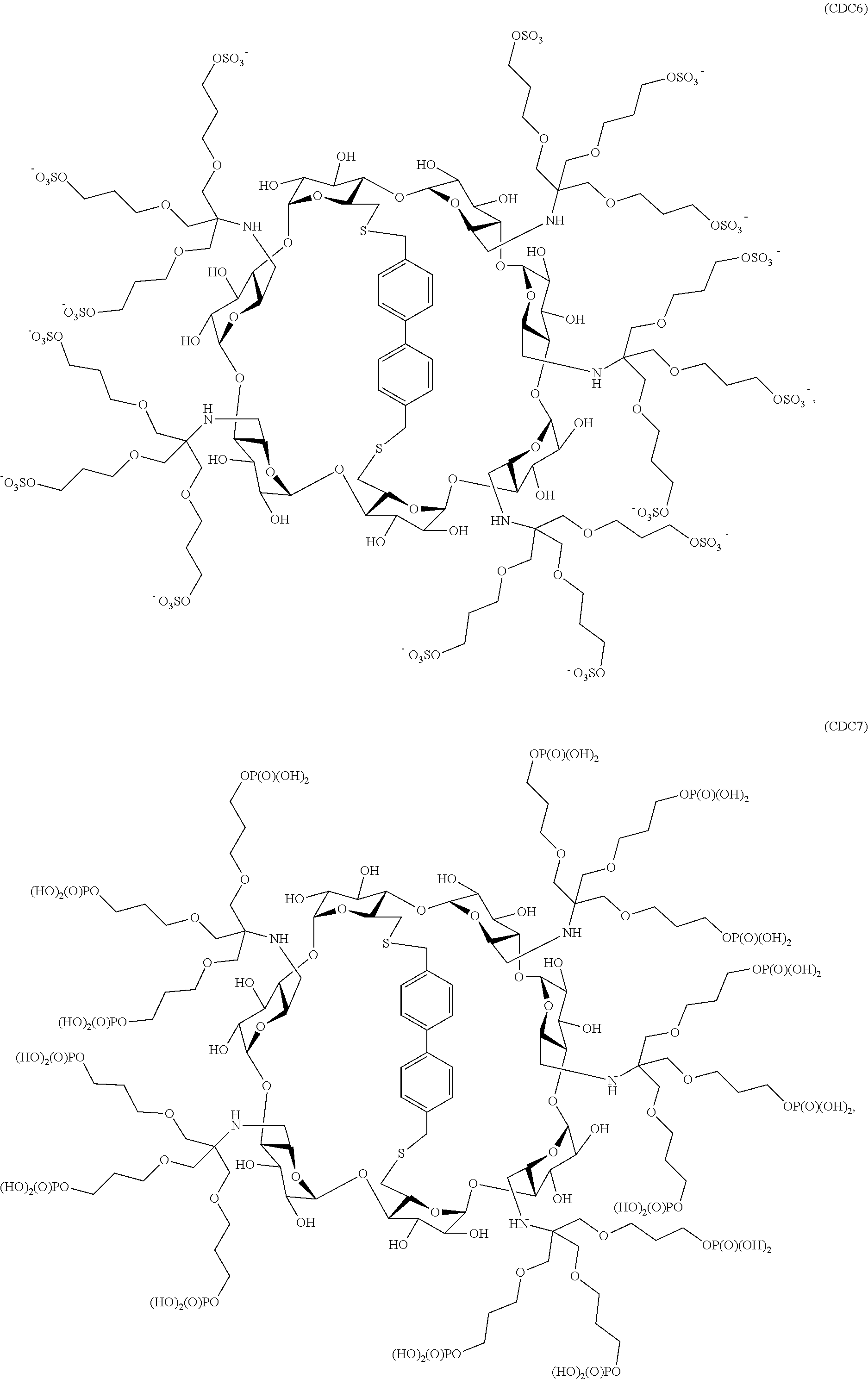 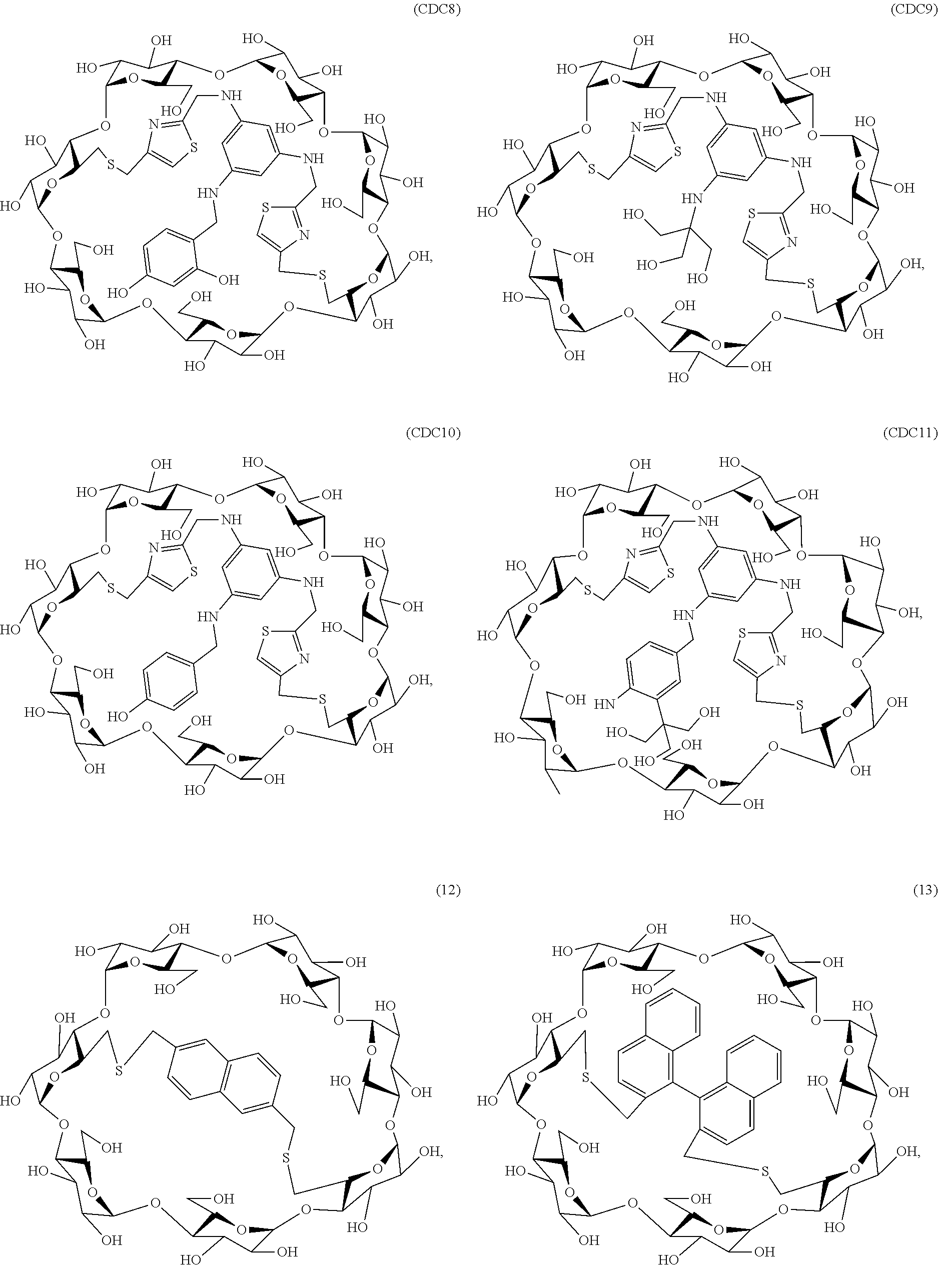 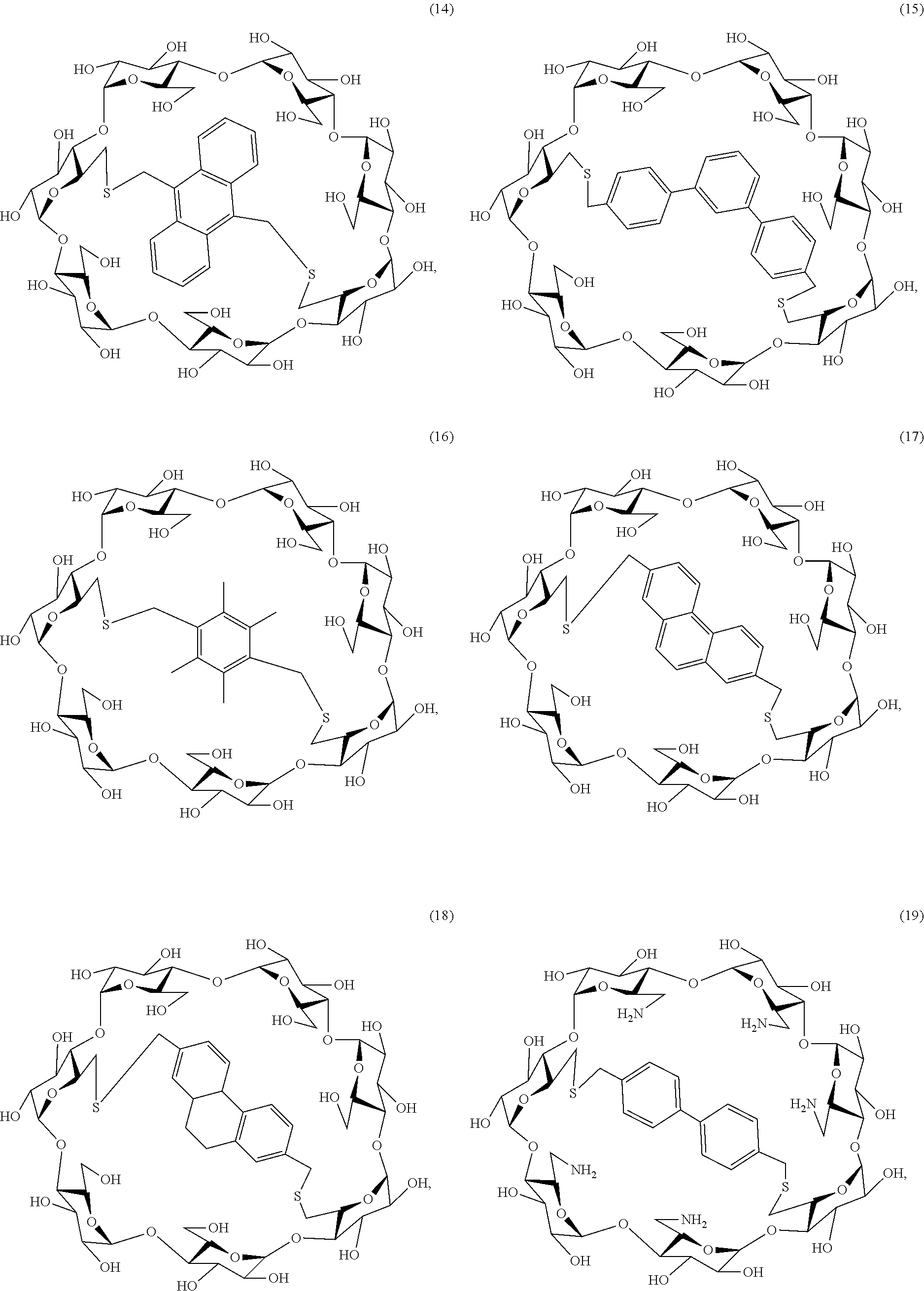 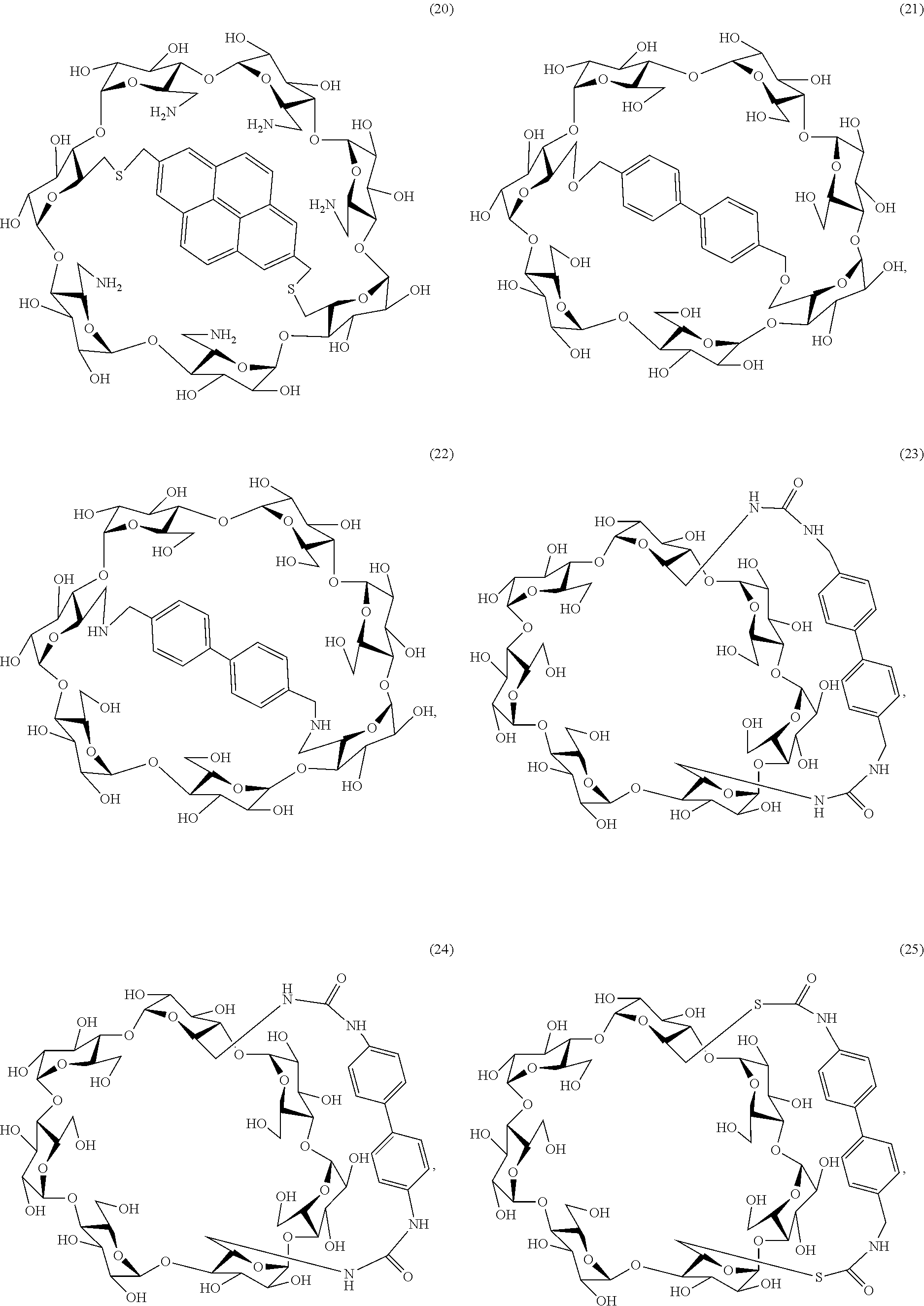 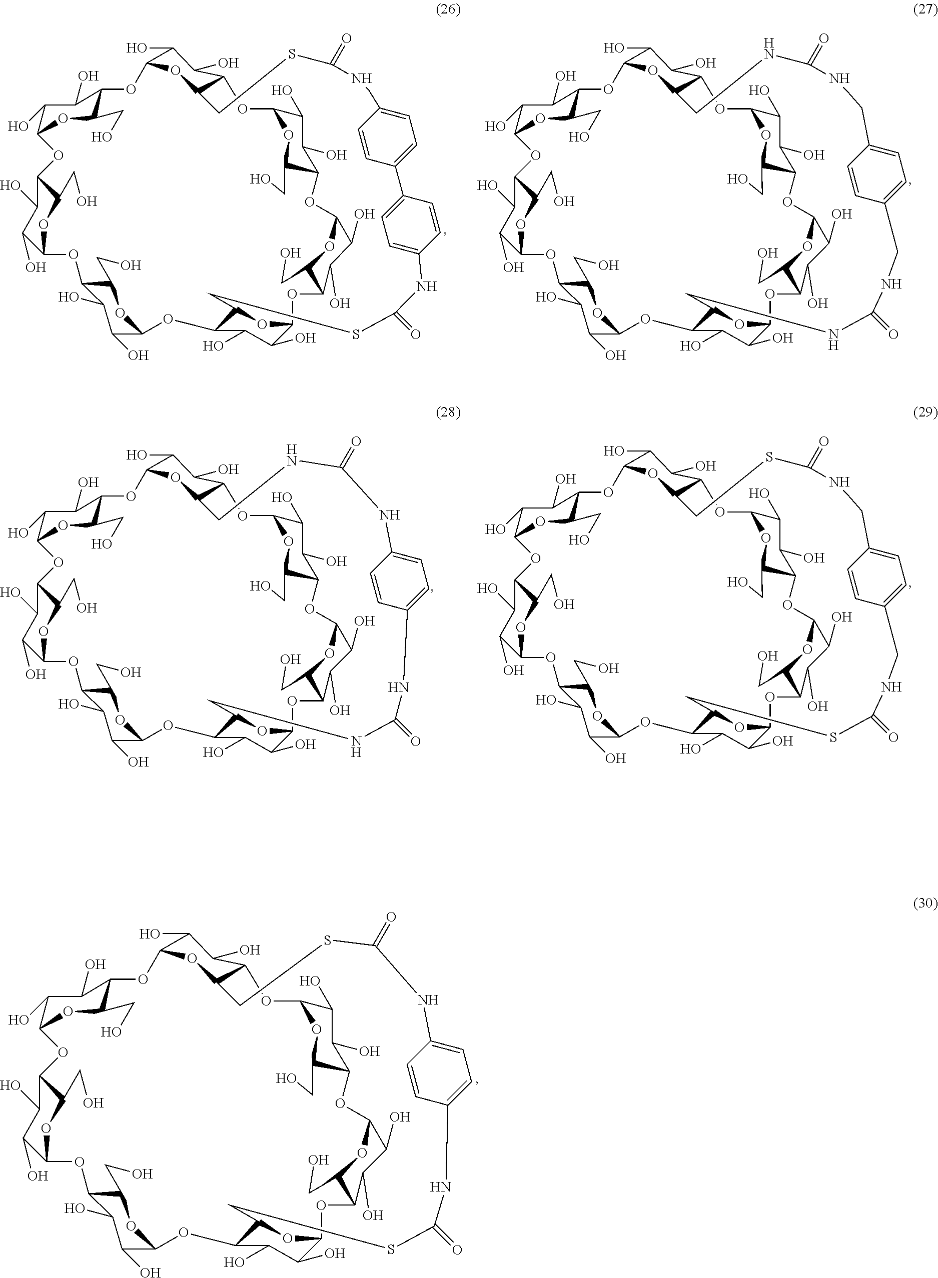 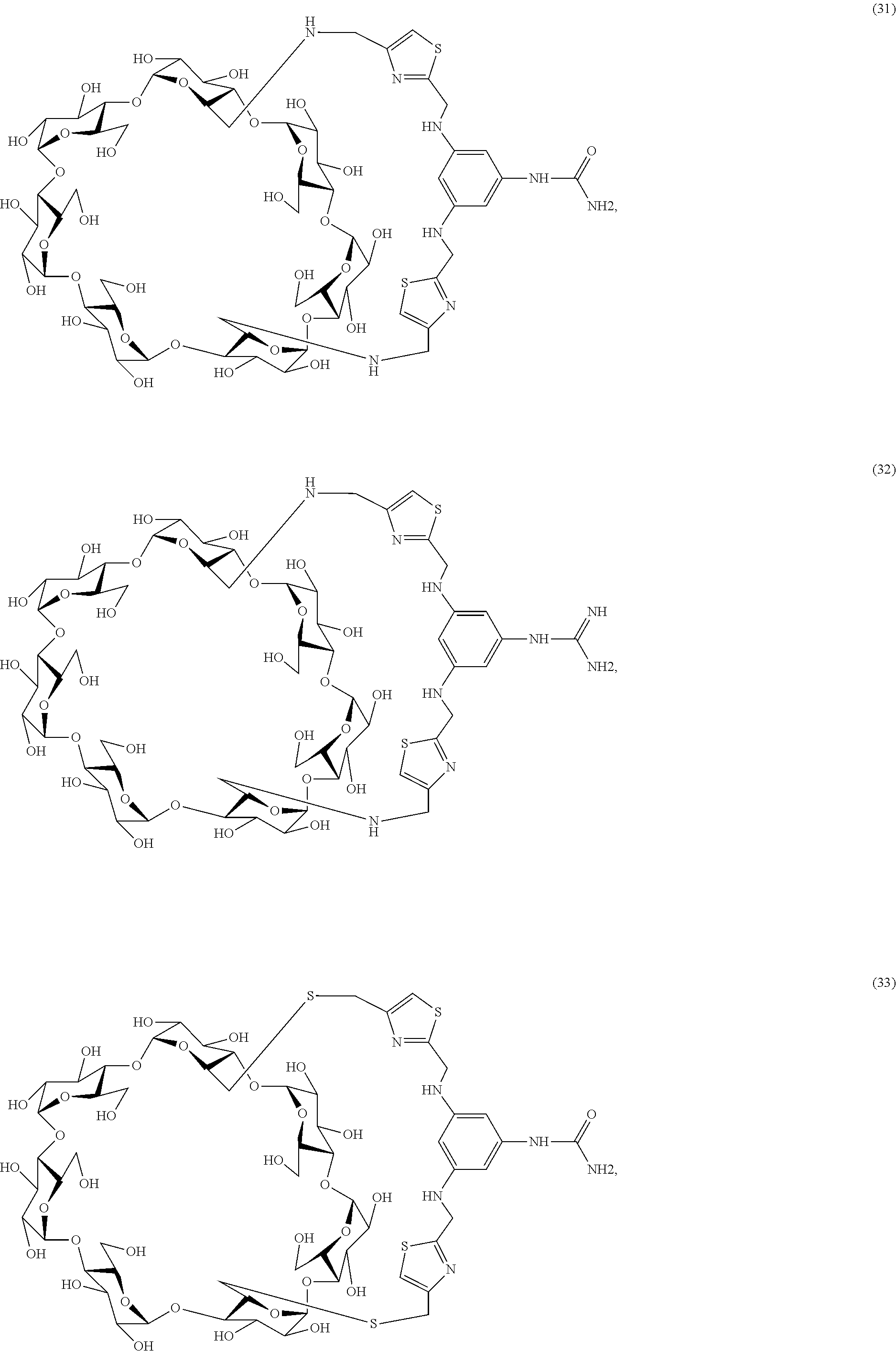 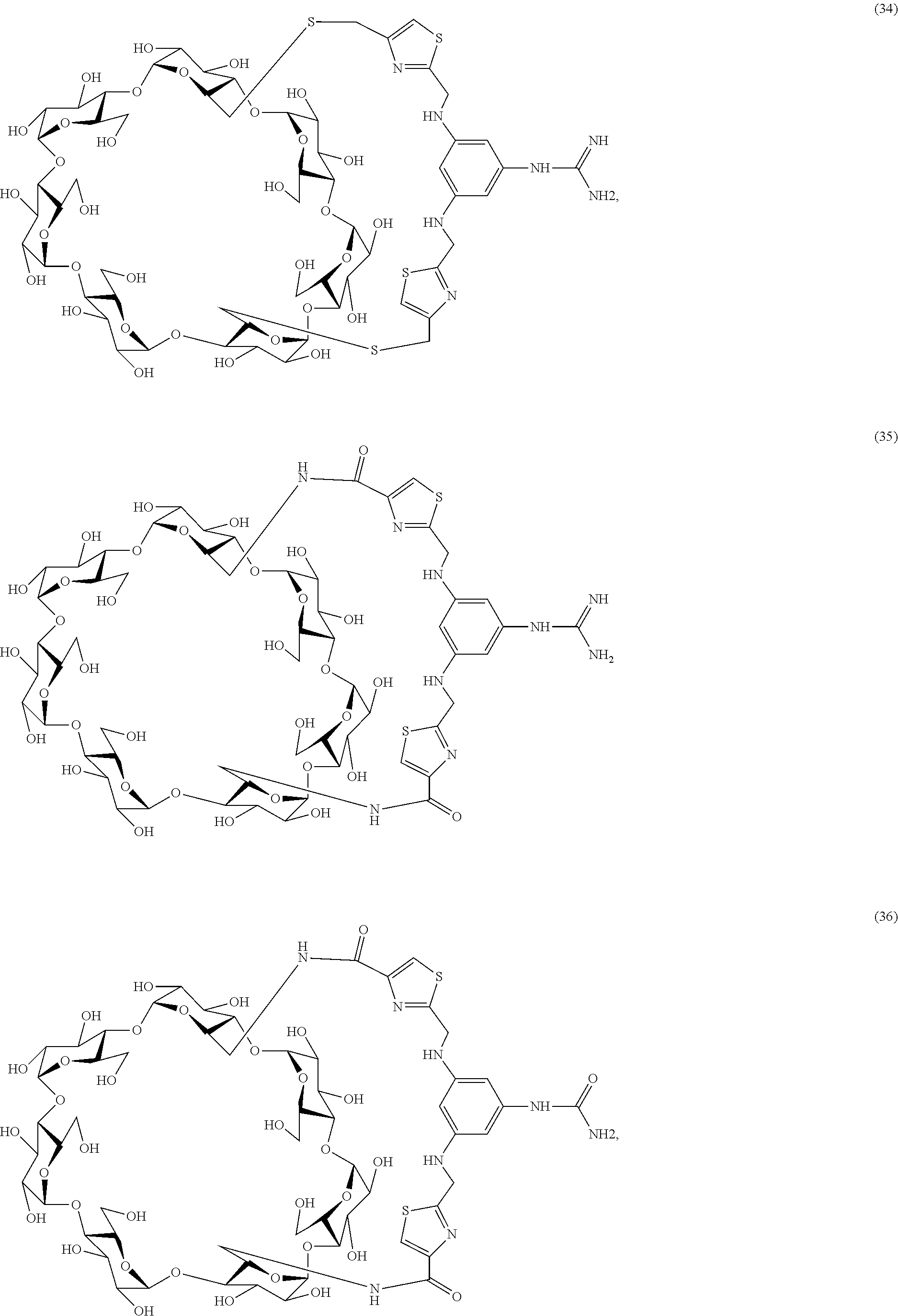 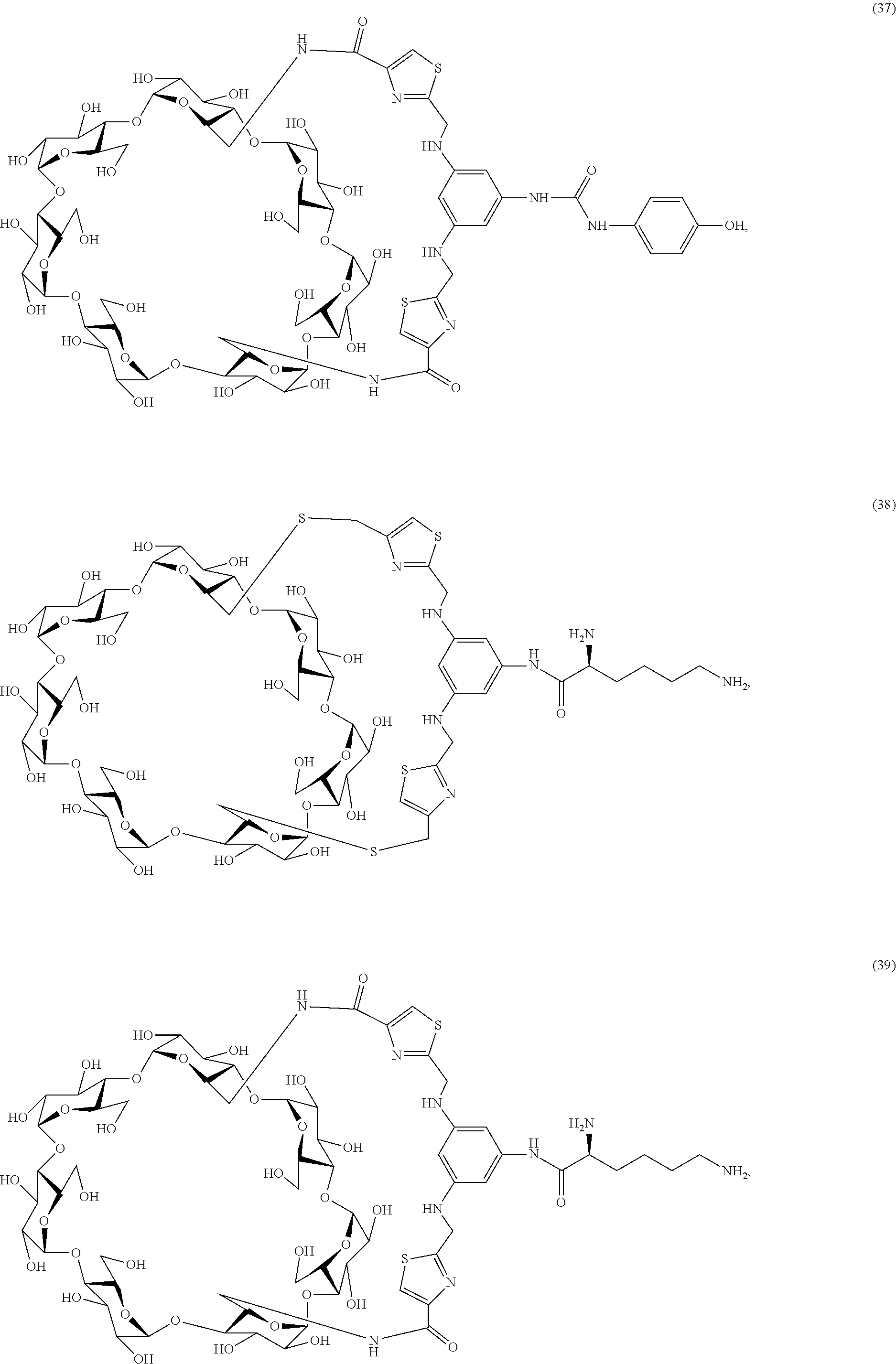 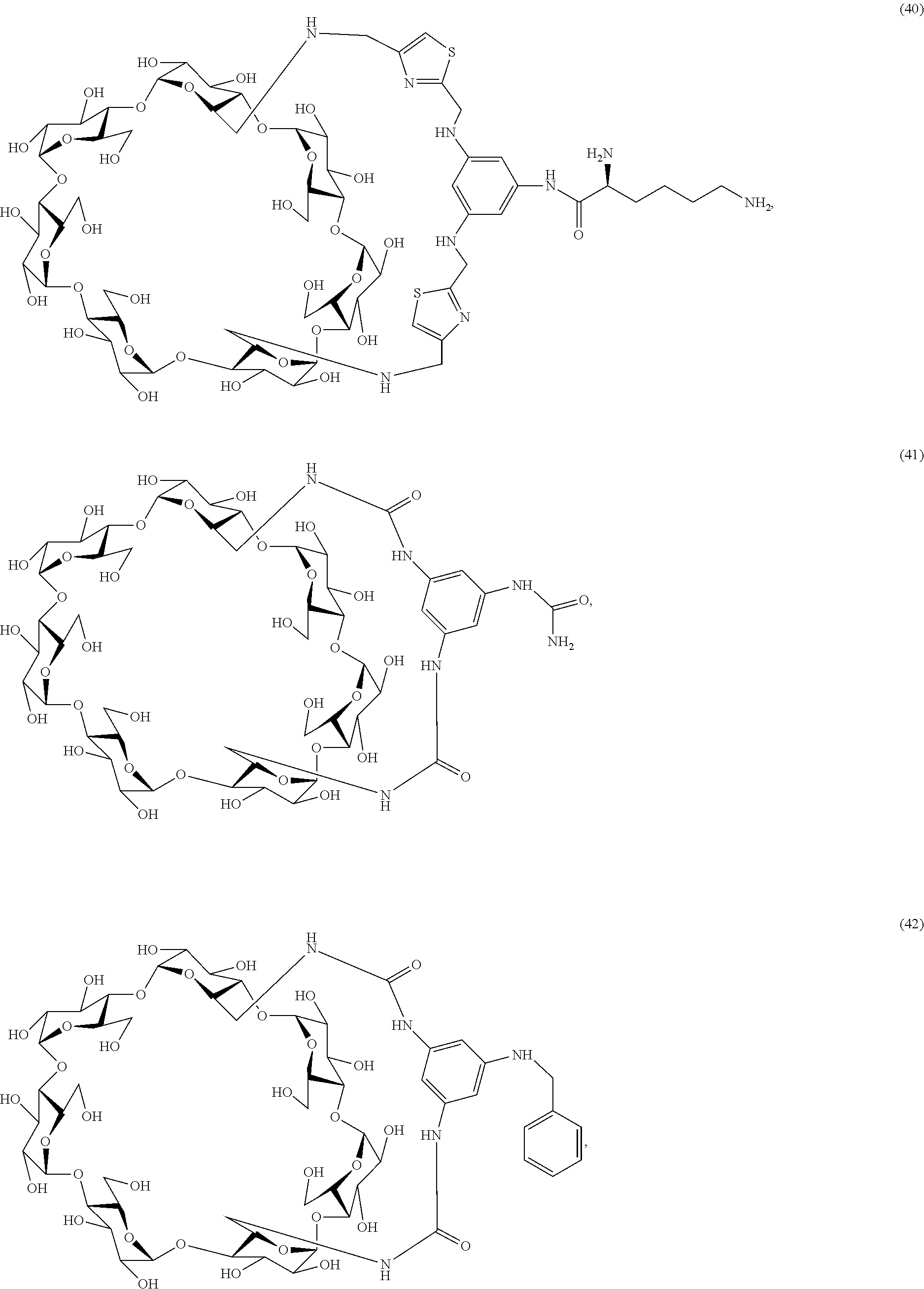 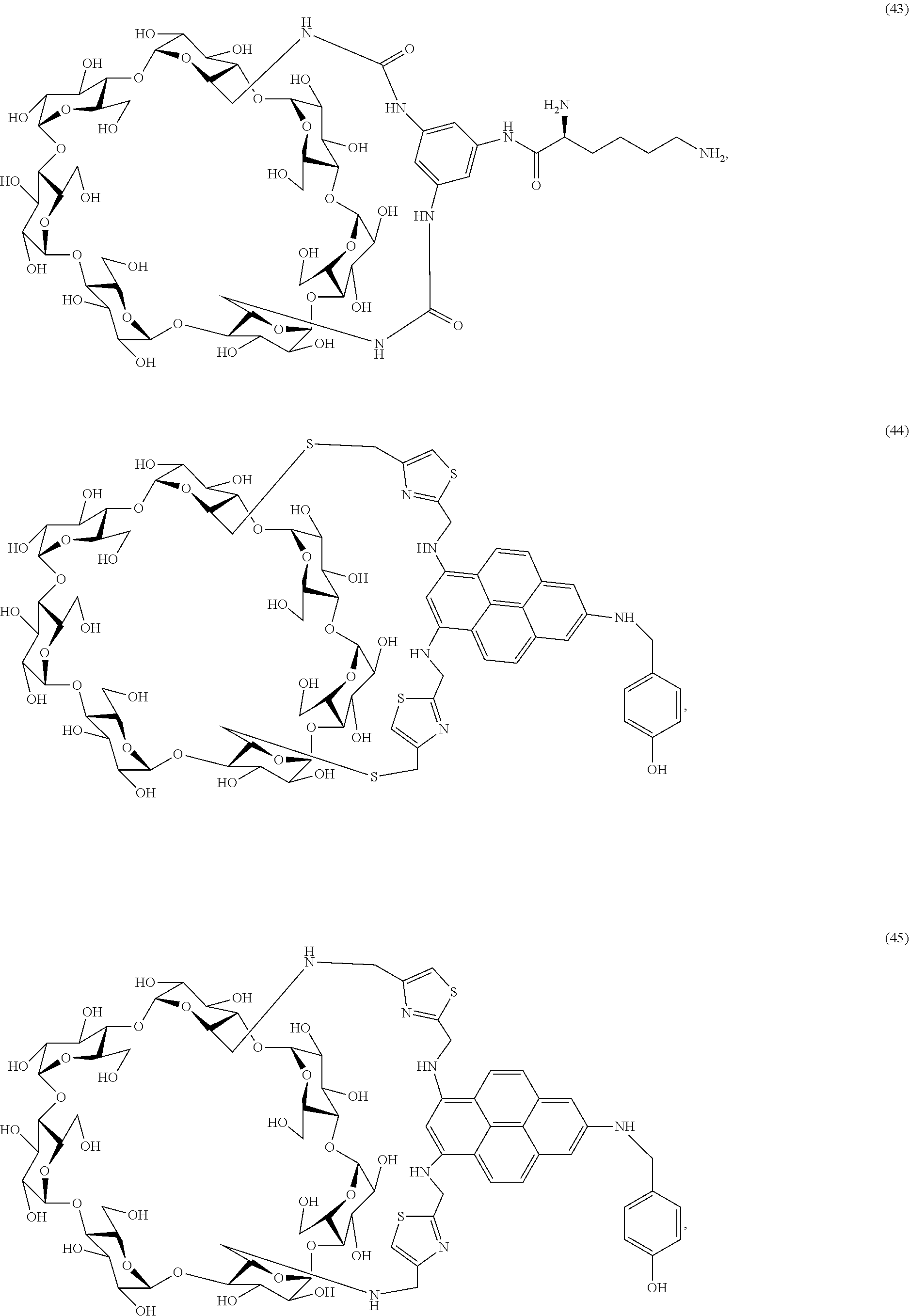 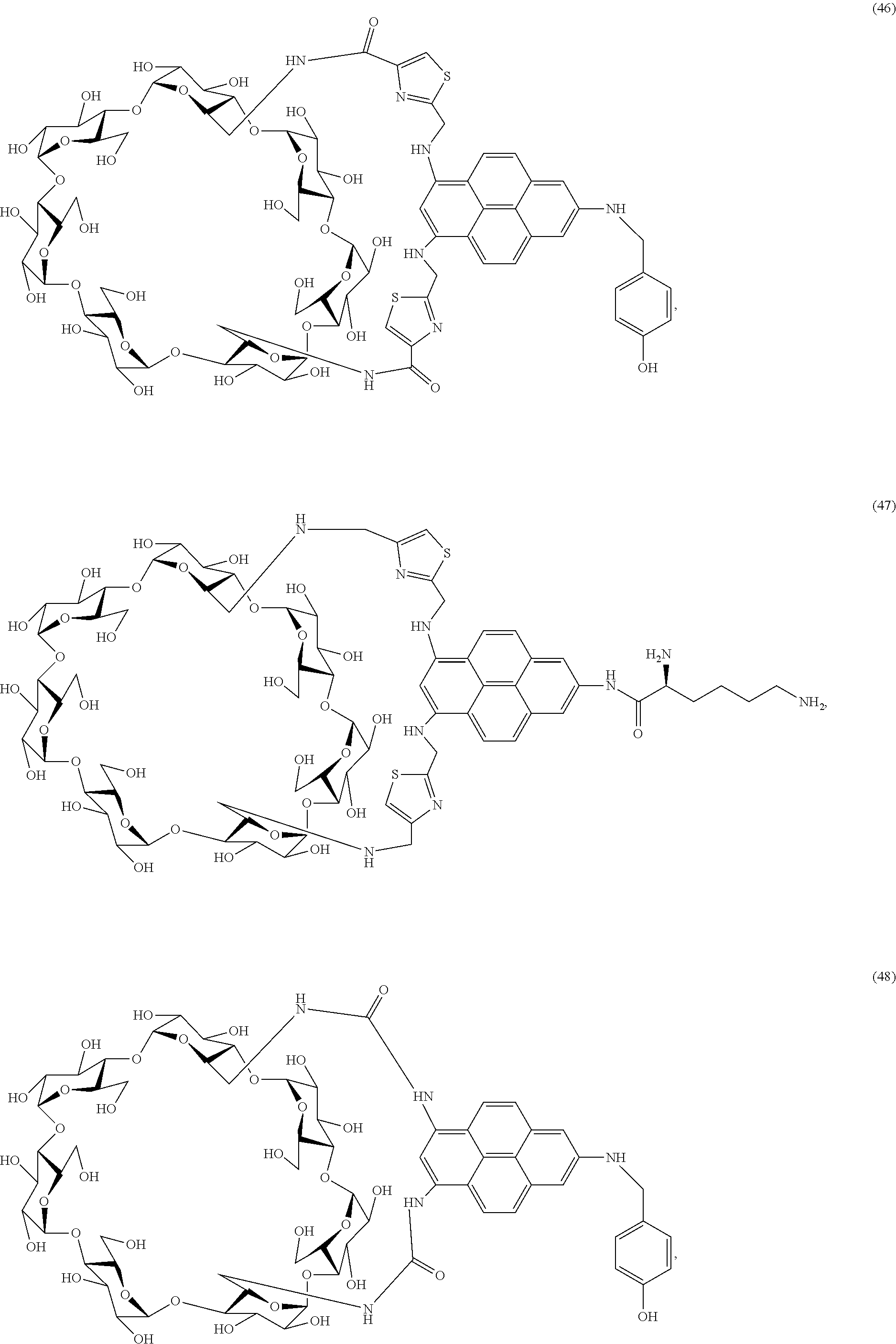 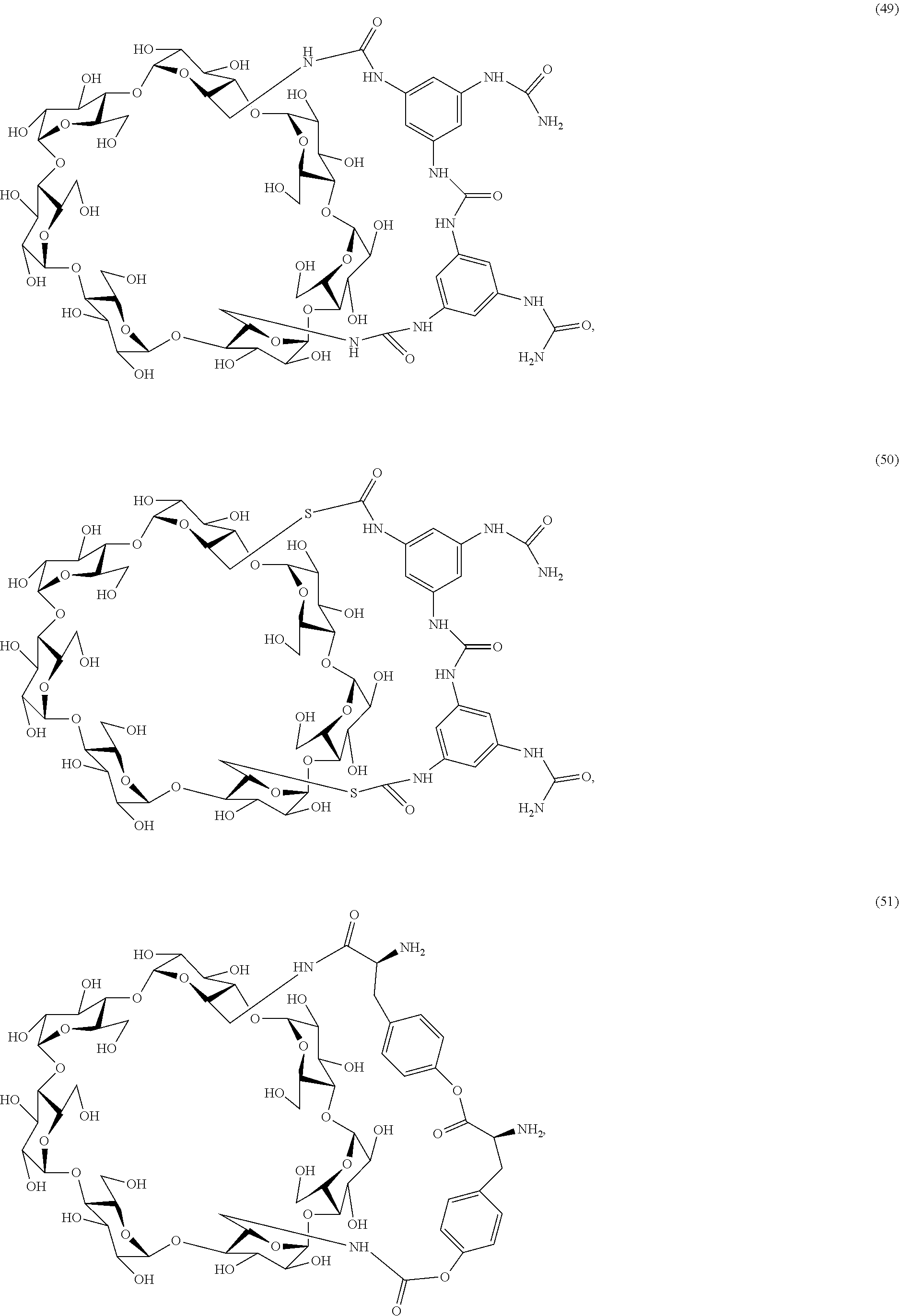 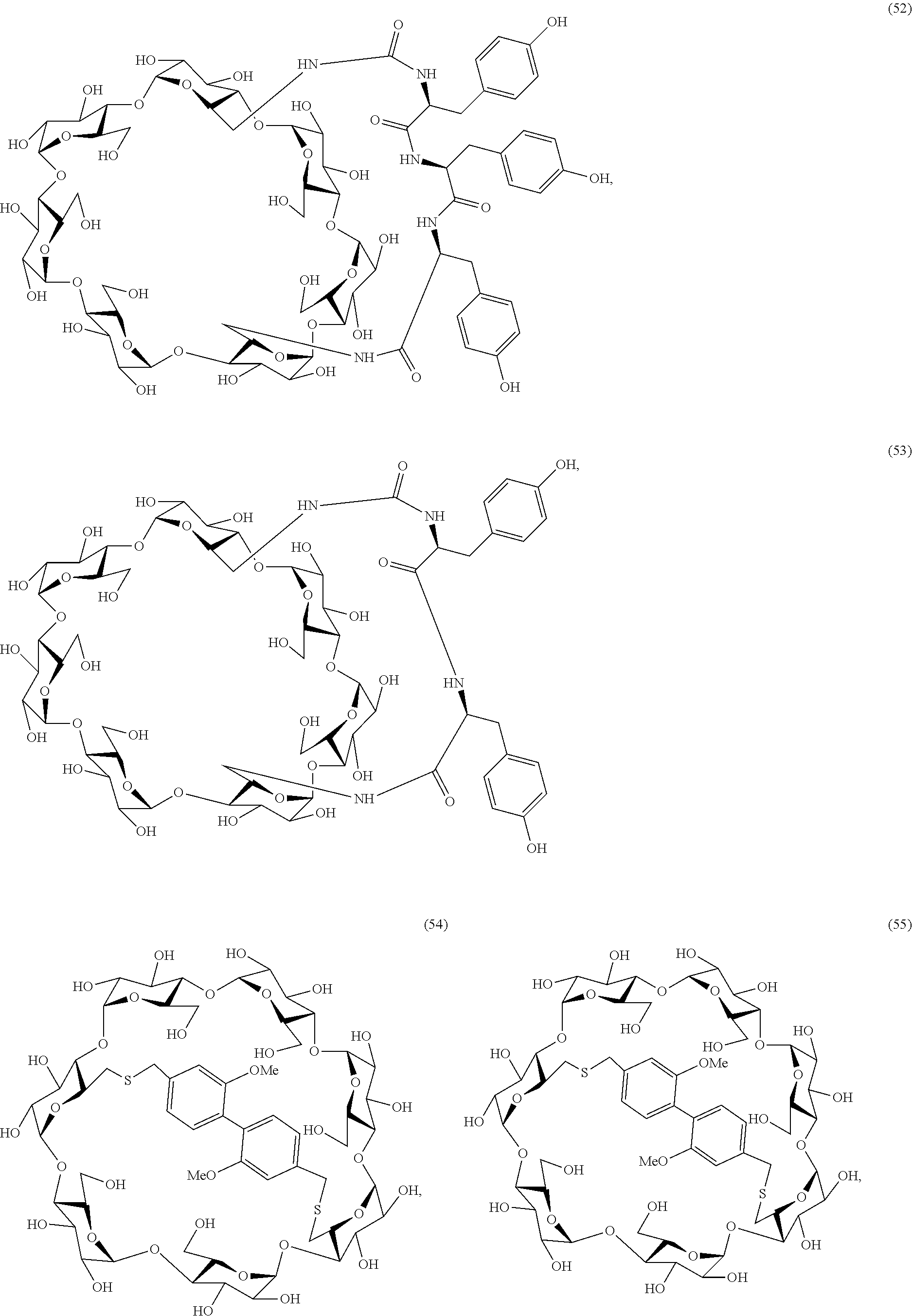 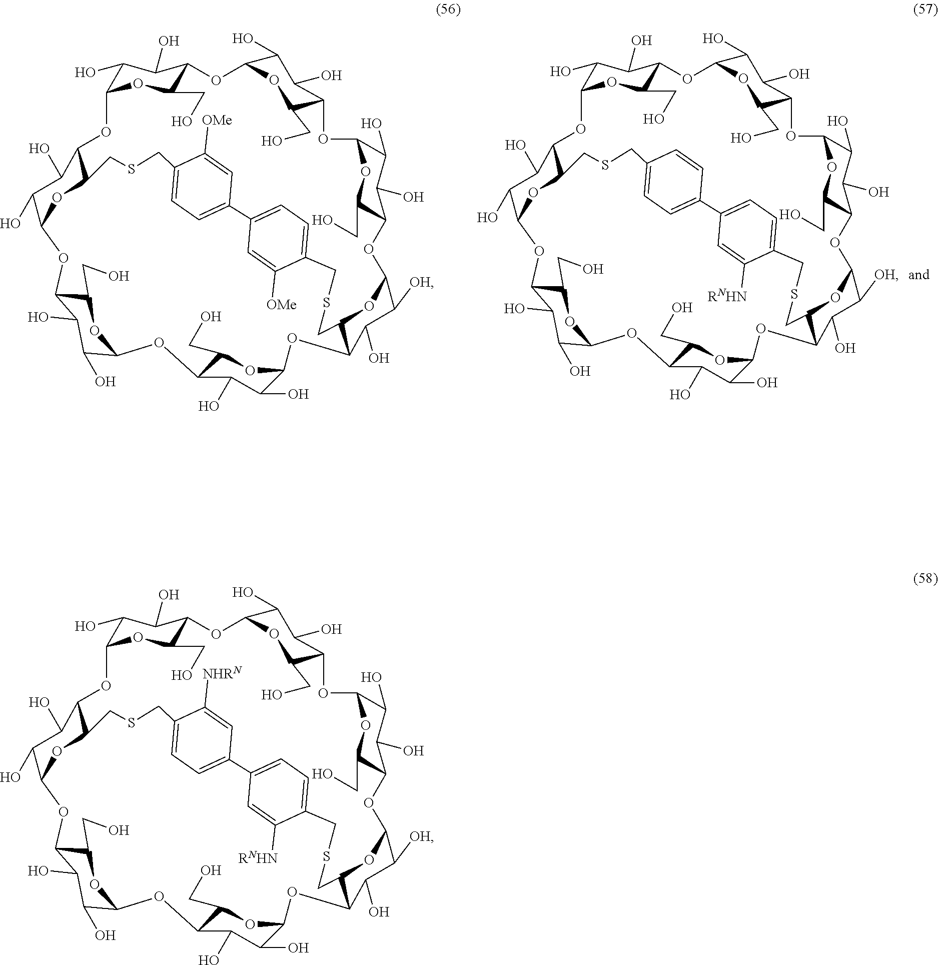
and pharmaceutically acceptable salts of; wherein each R^{N} is a group of one of the following formulae:

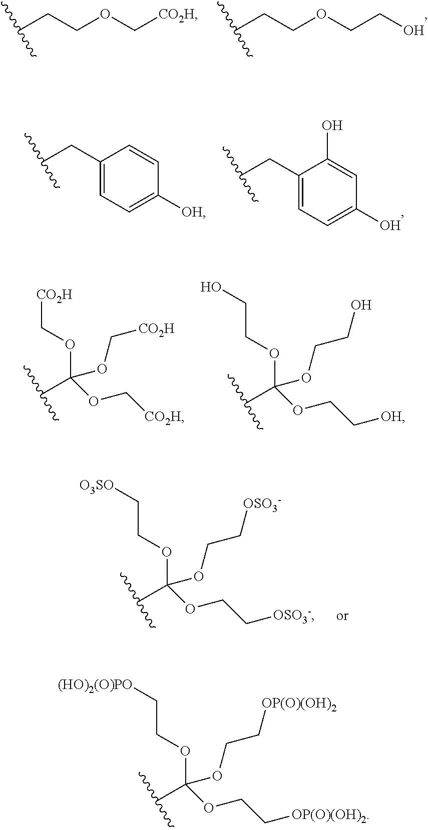

80. A conjugate comprising a compound of any one of claims 1-79, or a pharmaceutically acceptable salt thereof, wherein the compound is conjugated to an agent or a tag.

81. The conjugate of claim 80, or a pharmaceutically acceptable salt thereof, wherein the compound is conjugated to an agent.

82. The conjugate of claim 81, or a pharmaceutically acceptable salt thereof, wherein the agent is selected from the group consisting of proteins, peptides, nucleic acids, polysaccharides, lipids, and small molecules.

83. The conjugate of claim 81, or a pharmaceutically acceptable salt thereof, wherein the agent is a diagnostic agent, imaging agent, therapeutic agent, or prophylactic agent.

84. The conjugate of claim 82, or a pharmaceutically acceptable salt thereof, wherein the agent is a protein.

85. The conjugate of claim 82, or a pharmaceutically acceptable salt thereof, wherein the protein is a therapeutic protein.

86. The conjugate of claim 85, or a pharmaceutically acceptable salt thereof, wherein the protein is insulin.

87. The conjugate of claim 86, or a pharmaceutically acceptable salt thereof, wherein the insulin is wild-type insulin or modified insulin.

88. The conjugate of claim 80, or a pharmaceutically acceptable salt thereof, wherein the compound is conjugated to a tag.

89. The conjugate of claim 88, or a pharmaceutically acceptable salt thereof, wherein the tag is selected from the group consisting of radionuclides, fluorophores, chromophores, phosphorescent agents, dyes, chemiluminescent agents, colorimetric labels, magnetic labels, haptens, and excipients.

90. The conjugate of any one of claims 80-89, wherein the compound is of Formula (II): 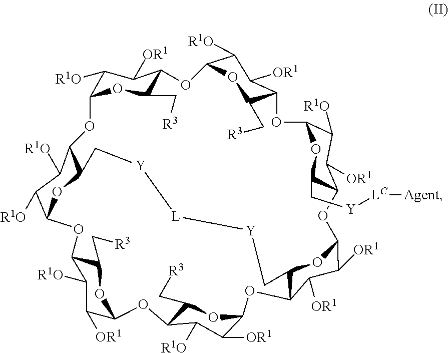
or a pharmaceutically acceptable salt thereof, wherein:
L^{C} is a linker; and
Agent is an agent selected from the group consisting of proteins, peptides, nucleic acids, polysaccharides, lipids, and small molecules.

91. The conjugate of claim 90, wherein the compound is of Formula (II-a): 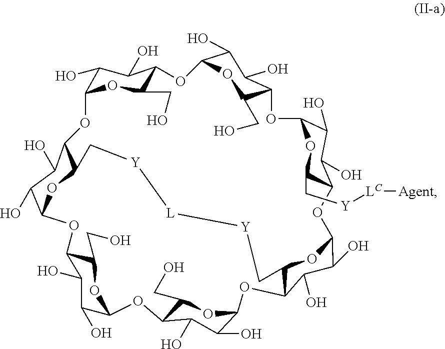
or a pharmaceutically acceptable salt thereof.

92. The conjugate of claim 90 or 91, or a pharmaceutically acceptable salt thereof, wherein L^{C} is a polymeric linker.

93. The conjugate of claim 92, or a pharmaceutically acceptable salt thereof, wherein L^{C} is a PEG linker.

94. The conjugate of any one of claims 90-93, or a pharmaceutica acce able salt thereof, wherein the Agent is a protein.

95. The conjugate of claim 94, or a pharmaceutically acceptable salt thereof, wherein the protein is a therapeutic protein.

96. The conjugate of claim 95, or a pharmaceutically acceptable salt thereof, wherein the protein is insulin.

97. The conjugate of claim 96, or a pharmaceutically acceptable salt thereof, wherein the insulin is wild-type insulin or modified insulin.

98. The conjugate of any one of claims 90-97, wherein the compound is of Formula (II-b): 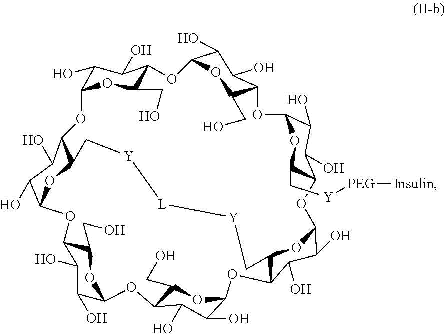
or a pharmaceutically acceptable salt thereof.

99. The conjugate of any one of claims 90-98, wherein the compound is of Formula (II-c): 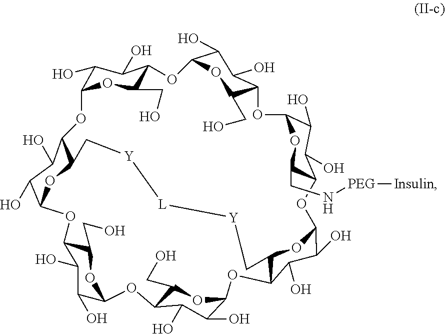
or a pharmaceutically acceptable salt thereof.

100. The conjugate of any one of claims 90-99, or a pharmaceutically acceptable salt thereof, wherein the compound is of Formula (II-d): 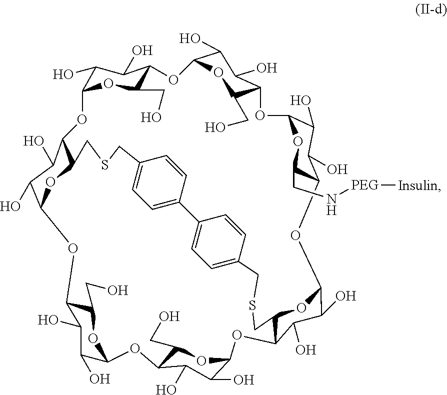
or a pharmaceutically acceptable salt thereof.

101. A complex comprising a compound of any one of claims 1-79, or a pharmaceutically acceptable salt thereof, or a conjugate of any one of claims 80-11, and an agent, wherein the compound is associated with the agent through non-covalent interactions.

102. The complex of claim 101, wherein the agent is a sugar.

103. The complex of claim 101 or 102, wherein the agent is glucose.

104. A composition comprising a compound of any one of claims 1-79, or a pharmaceutically acceptable salt thereof, or a conjugate of any one of claims 80-100.

105. A composition comprising a complex of any one of claims 101-103, or a pharmaceutically acceptable salt thereof.

106. A method of sensing or detecting glucose comprising contacting glucose with a compound of any one of claims 1-79, or a pharmaceutically acceptable salt thereof, or a conjugate of any one of claims 80-100, or a composition of claim 104.

107. The method of claim 106, wherein the contacting occurs in vitro.

108. The method of claim 106, wherein the contacting occurs in vivo.

109. The method of claim 108 further comprising a step of administering a compound of any one of claims 1-79, or a pharmaceutically acceptable salt thereof, or a conjugate of any one of claims 80-100, to a subject.

110. The method of claim 109, wherein the subject is a human.

111. A method for treating or diagnosing a disease in a subject, the method comprising administering to the subject a compound of any one of claims 1-79, or a pharmaceutically acceptable salt thereof, or a conjugate of any one of claims 80-100, or a composition of claim 104.

112. . The method of claim 111, wherein the disease is a metabolic disorder.

113. The method of claim 111 or 112, wherein the disease is diabetes.

114. The method of any one of claims 111-113, wherein the disease is type 1 or type 2 diabetes.

115. A method of delivering insulin to a subject, the method comprising administering to the subject a compound of any one of claims 1-79, or a pharmaceutically acceptable salt thereof, or a conjugate of any one of claims 80-100, or a composition of claim 104.

116. The method f any one of claims 111-115, wherein the subject is a human.

117. A kit comprising a compound, conjugate, or composition of any one of the preceding claims.
